(19) United States
(12) Patent Application Publication
(10) Pub. No.: US 2012/0041019 A1
(43) Pub. Date: Feb. 16, 2012

(54) PROTEASE INHIBITORS

(76) Inventors: Mitchell W. Mutz, La Jolla, CA (US); Kenneth J. Barr, Boston, MA (US); Jason Gestwicki, Ann Arbor, MI (US)

(21) Appl. No.: 13/139,503

(22) PCT Filed: Dec. 17, 2009
(86) PCT No.: PCT/US09/06600

§ 371 Date: Sep. 27, 2011

Related U.S. Application Data
(60) Provisional application No. 61/138,428, filed on Dec. 17, 2008.

(51) Int. Cl. A61K 31/4525; A61K 31/445; C07D 498/16; A61K 31/436; A61P 31/14; A61K 31/4439; A61K 31/4545; C07D 405/12; A61P 31/18; C07D 211/60; C07D 401/12

(52) U.S. Cl. 514/291; 546/226; 514/330; 546/90; 546/279.1; 514/343; 546/193; 514/318; 546/214; 514/326; 546/194

(57) ABSTRACT

Compounds useful as protease inhibitors are provided, as are methods of use and preparation of such compounds and compositions containing such compounds. In one embodiment, the compounds are useful for inhibiting HIV protease enzymes, and are therefore useful in slowing the proliferation of HIV.

### CROSS REFERENCE TO RELATED APPLICATIONS

This application claims priority under 35 U.S.C. §119(e) to provisional U.S. application Ser. No. 61/138,428, filed Dec. 17, 2008, the entire contents of which is incorporated herein by reference.

### TECHNICAL FIELD

The present disclosure relates to compounds useful for inhibiting protease enzymes, as well as methods of use and methods of manufacture of such compounds. The disclosure finds utility, for example, in the field of pharmacology.

### BACKGROUND

A wide range of diseases are caused by retroviruses. As presently understood, acquired immunodeficiency syndrome (AIDS) is a disease of the immune system caused by the retrovirus HIV (Human Immunodeficiency Virus). According to estimates from the World Health Organization, AIDS affects millions of people and is continuing to spread. In virtually all cases, AIDS causes a gradual breakdown of the body's immune system as well as progressive deterioration of the central and peripheral nervous systems.

The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases such as, among others, pneumocystic carini pneumonia, Kaposi's sarcoma, and cancer of the lymph system.

Retroviral replication routinely features post-translational processing of polyproteins. This yields mature polypeptides that will subsequently aid in the formation and function of infectious virus. In the case of HIV, this post-translational processing is accomplished by virally encoded HIV protease enzyme. A retroviral protease is a proteolytic enzyme that participates in the maturation of new infectious virions in infected cells during the reproductive cycle. Interruption of the normal viral reproduction cycle can be affected by disrupting the protease enzyme. Therefore, inhibitors of HIV protease may function as anti-HIV viral agents.

On-going treatment of HIV-infected individuals with compounds that inhibit HIV protease has led to the development of mutant viruses that possess proteases that are resistant to the inhibitory effect of these compounds. Thus, to be effective, it is desirable that new HIV protease inhibitors are effective not only against wild-type strains of HIV, but also against the newly emerging mutant strains that are resistant to the commercially available protease inhibitors.

Some antiviral compounds that act as HIV protease inhibitors are described in WO 99/67254. Known HIV protease inhibitors include: saquinavir; ritonavir; indinavir; nelfinavir; amprenavir; lopinavir; atazanavir; fosamprenavir; tipranavir; and darunavir. Common methods of treatment using HIV protease inhibitors include co-administration and co-dosing with a plurality of these compounds. For example, ritonavir is frequently administered along with other of these HIV protease inhibitors.

In addition to the problematic development of strains of the virus resistant to known inhibitors, some HIV protease inhibitors are difficult to prepare, are expensive to obtain, and/or have significant adverse side effects; all of these drawbacks may result in lower patient compliance and less effective treatment. Accordingly, there continues to be a need for the development of new inhibitors effective to inhibit the HIV protease in both wild type and mutant strains of HIV.

### SUMMARY OF THE DISCLOSURE

The present disclosure provides compounds that address one or more of the abovementioned drawbacks. In particular, the present disclosure provides compounds useful as protease inhibitors.

In one embodiment, the disclosure provides compounds having the structure of formula (A)

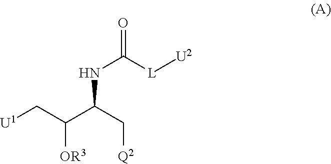

wherein: Q² is selected from alkyl and aryl; R³ is selected from H, hydrocarbyl, functional groups, hydroxyl-protecting groups, and inorganic acid groups; U¹ is selected from hydrocarbyl and functional groups; L is a linking moiety selected from hydrocarbylene and functional groups; U² is a group selected from Units A, and B:

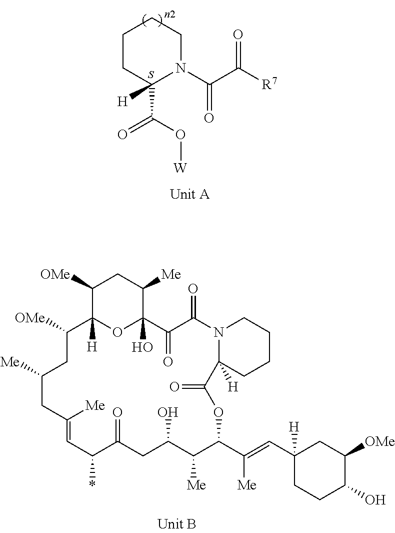

wherein: n2 is an integer from 0 to 2; R⁷ is selected from H, hydrocarbyl, and functional groups; W is a direct bond to L or is a linker selected from alkylene, arylene, and

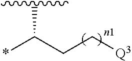

wherein the wavy line represents the attachment point to Unit A, n1 is an integer selected from 1 and 2, and Q³ is selected from aryl and alkyl; and the stars represent the point of connection to L, as well as pharmaceutically acceptable salts, prodrugs, and metabolites thereof.

In further embodiments, the disclosure provides compounds having the structure of formula (B)

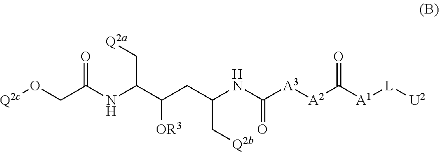

wherein: A¹ and A² are independently selected from nitrogen-containing linking moieties; A³ is a hydrocarbylene linker; Q^{2a}, Q^{2b}; and Q^{2c} are independently selected from alkyl and aryl; R³ is selected from H, hydrocarbyl, functional groups, hydroxyl-protecting groups, and inorganic acid groups; L is a linking moiety selected from hydrocarbylene and functional groups; U² is a group selected from Units A, and B, as shown above; as well as pharmaceutically acceptable salts, prodrugs, and metabolites thereof.

In still further embodiments, the disclosure provides a pharmaceutical formulation comprising a compound selected from those having the structure of formula (A) or formula (B) and a pharmaceutically acceptable carrier.

In still further embodiments, the disclosure provides a method for treating a patient with a protease inhibitor comprising administering an effective amount of a compound selected from those having the structure of formula (A) or formula (B).

In still further embodiments, the disclosure provides a method of synthesizing any of the compounds disclosed herein. The method comprising coupling a core fragment and an additional unit to a linker moiety.

### BRIEF DESCRIPTION OF THE DRAWINGS

FIG. 1 provides a chart showing the IC₅₀ values in cell free assays for several compounds according to the disclosure.

FIG. 2 provides a chart showing the IC₅₀ values in cell infectivity assays for several compounds according to the disclosure.

### DETAILED DESCRIPTION OF THE INVENTION

Unless otherwise indicated, the disclosure is not limited to specific procedures, starting materials, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a reactant” includes not only a single reactant but also a combination or mixture of two or more different reactant, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties. Similarly, “hydrocarbylene” refers to a hydrocarbyl di-radical group, such as a hydrocarbylene linker group.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂), mono- and di-(C₁-C₂₄ alkyl)-substituted phosphino, mono- and di-(C₅-C₂₀ aryl)-substituted phosphino; and the hydrocarbyl moieties C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

It will be appreciated that many chemical moieties fall within more than one of the above definitions. For example, a benzyl group can at least be classified as a substituted alkyl group, a substituted or unsubstituted aralkyl group, and a substituted or unsubstituted hydrocarbyl group. Unless otherwise indicated, when defining variables for the chemical formulae herein, recitation of one class of moieties is not intended to exclude those moieties that also fall within a class not recited. For example, the phrase “R is alkyl” includes (unless otherwise indicated) substituted alkyl, and does not exclude benzyl even though “aralkyl” is not also included in the phrase.

By the term “linker group,” “linker moiety,” or “linker” is meant a di-radical group that connects two portions of a compound. For example, hydrocarbylene, alkylene, aralkylene, etc. are “linker groups.” As another example, a “functional linker group” is a functional group (as defined above) that is a di-radical, and connects two portions of a compound. For example, oxo (—O—) and amido (—NH—) groups are functional linker groups.

When the term “substituted” appears prior to a list of possible substituted groups, it is intended that the term apply to every member of that group. For example, the phrase “substituted alkyl and aryl” is to be interpreted as “substituted alkyl and substituted aryl.”

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Unless otherwise indicated, the terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, the terms include prophylactic use of active agents. “Preventing” a disorder or unwanted physiological event in a patient refers specifically to the prevention of the occurrence of symptoms and/or their underlying cause, wherein the patient may or may not exhibit heightened susceptibility to the disorder or event.

By the term “effective amount” of a therapeutic agent is meant a nontoxic but sufficient amount of a beneficial agent to provide a desirable effect.

As used herein, and unless specifically stated otherwise, an “effective amount” of a beneficial refers to an amount covering both therapeutically effective amounts and prophylactically effective amounts.

As used herein, a “therapeutically effective amount” of an active agent refers to an amount that is effective to achieve a desired therapeutic result, and a “prophylactically effective amount” of an active agent refers to an amount that is effective to prevent or lessen the severity of an unwanted physiological condition.

By a “pharmaceutically acceptable” component is meant a component that is not biologically or otherwise undesirable, i.e., the component may be incorporated into a pharmaceutical formulation of the disclosure and administered to a patient as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “pharmacologically active” (or simply “active”), as in a “pharmacologically active” derivative or analog, refers to a derivative or analog (e.g., a salt, ester, amide, conjugate, metabolite, isomer, fragment, etc.) having the same type of pharmacological activity as the parent compound and approximately equivalent in degree.

The term “controlled release” refers to a formulation, dosage form, or region thereof from which release of a beneficial agent is not immediate, i.e., with a “controlled release” dosage form, administration does not result in immediate release of the beneficial agent in an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a formulation, dosage form, or region thereof that provides for gradual release of a beneficial agent over an extended period of time, and that preferably, although not necessarily, results in substantially constant blood levels of the agent over an extended time period.

The term “naturally occurring” refers to a compound or composition that occurs in nature, regardless of whether the compound or composition has been isolated from a natural source or chemically synthesized.

As used herein, the term “protease inhibitor” refers to compounds that inhibit proteases of viral origin, and that are useful in the treatment of viral infections caused by retroviruses, such as HIV, in mammals, both human and nonhuman.

In a first embodiment, the disclosure provides compounds having the structure of formula (A)

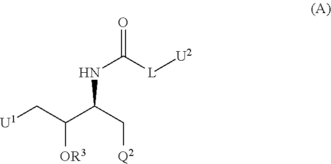

wherein:

Q² is selected from alkyl and aryl;

R³ is selected from H, hydrocarbyl, functional groups, hydroxyl-protecting groups, and inorganic acid groups;

U¹ is selected from hydrocarbyl and functional groups;

L is a linking moiety selected from hydrocarbylene and functional groups;

U² is a group selected from Units A, and B:

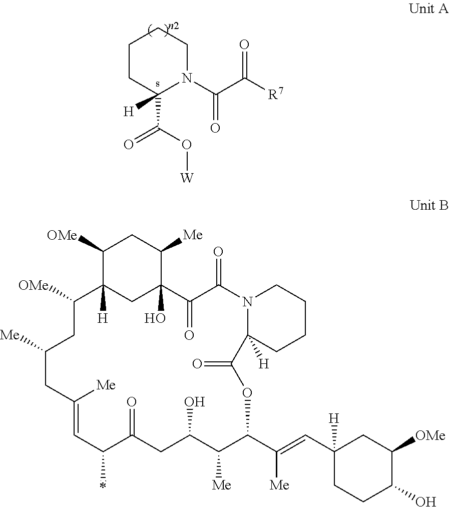

wherein:

n2 is an integer from 0 to 2;

R⁷ is selected from H, hydrocarbyl, and functional groups;

W is a linker that links Unit A with L, and is selected from a bond, an alkylene group, an arylene group, and

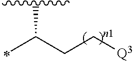

wherein the wavy line represents the attachment point to Unit A, n1 is an integer selected from 1 and 2, and Q³ is selected from aryl and alkyl; and

the stars represent the point of connection to L, as well as pharmaceutically acceptable salts, prodrugs, and metabolites thereof.

In some embodiments, U¹ is selected from

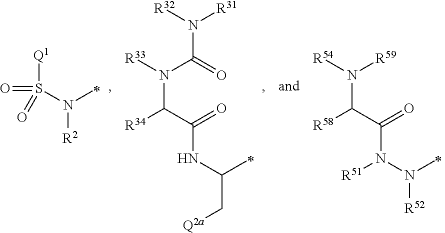

wherein:

the stars represent connection points to the remainder of formula (I);

Q¹ is selected from an aromatic group, an alicyclic group, and an amine group;

Q^{2a} is selected from substituted or unsubstituted alkyl, substituted or unsubstituted heteroatom-containing alkyl, substituted or unsubstituted aryl, and substituted or unsubstituted heteroaryl;

R² is selected from H, hydrocarbyl, and functional groups;

R³¹, R³², R³³, and R³⁴ are independently selected from H and hydrocarbyl, and wherein any two of R³¹, R³², R³³, and R³⁴ may be taken together to form a ring;

R⁵¹ is selected from H and alkyl;

R⁵² is selected from alkyl, aryl, aralkyl, and alkaryl;

R⁵⁴ is selected from —C(═O)-A-R⁵⁰ and Ar⁵;

A is selected from a bond, —O—, and —NR⁵⁵—;

R⁵⁰ is alkyl;

Ar⁵ is aryl;

R⁵⁵ is H or lower alkyl;

R⁵⁸ is alkyl, aryl, aralkyl, or alkaryl;

R⁵⁹ is H or alkyl, and wherein any two of R⁵¹, R⁵², R⁵⁴, R⁵⁸, and R⁵⁹ may be taken together to form a ring.

In some embodiments, R⁷ is branched alkyl.

In some embodiments, W is selected from a bond, unsubstituted alkylene, substituted alkylene (including branched alkylene), heteroatom-containing alkylene, substituted heteroatom containing alkylene, unsubstituted arylene, substituted arylene, heteroarylene, and substituted heteroarylene. For example, in some embodiments W is —(CH₂)ₙ—NH—, wherein n is an integer from 1 to 3. For example, in some embodiments, W is

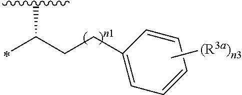

wherein n3 is an integer from 0 to 5 and each R^{3a} is independently selected from alkyl, alkoxy, halo, and functional groups. For example, W is

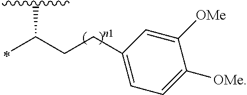

In some embodiments, compounds of formula (A) have the structure of formula (Ia)

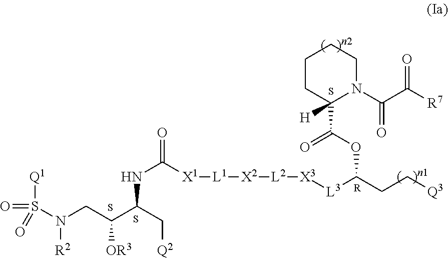

wherein, in formula (Ia):

X¹ is selected from a bond, —O—, and —NR¹⁰—, wherein R¹⁰ is selected from H and lower alkyl;

L¹ is selected from alkylene, arylene, alkarylene, and aralkylene;

X² is selected from a bond and —NR¹¹—, wherein R¹¹ is selected from H and lower alkyl;

L² is alkylene;

X³ is selected from —O—, and —NR¹²—, wherein R¹² is selected from H and lower alkyl, and

L³ is selected from an arylene group and an alkylene group.

For example, Q¹ is selected from substituted or unsubstituted C₅-C₃₀ aryl, substituted or unsubstituted C₅-C₃₀ heteroaryl, and —N(R¹³)(R¹⁴), wherein R¹³ and R¹⁴ are independently selected from H, lower alkyl, C₅-C₁₂ aryl, and C₅-C₁₂ heteroaryl, and further wherein R¹³ and R¹⁴ may be taken together to form a cycle. Examples of Q¹ include furan, thiophene, and thiazole, piperazine, piperidine, pyrrolidine, morpholine, benzo[c][1,2,5]oxadiazole, and other aryl or heteroaryl groups, any of which may be unsubstituted or substituted. Examples of preferred such substituents include halo, hydroxy, lower alkyl, lower alkoxy, amino, amido, acetamido, nitro, alkylcarbonyl, aryl, heteroaryl, and combinations thereof. For example, Q¹ can have the structure of formula Q^{1a}

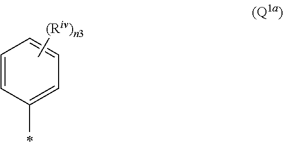

wherein:

the star represents the point of attachment to the remainder of the compound; and

each R^{iv} is independently selected from H, alkyl, aryl, heteroaryl, halo, alkoxy, amido, acetamido, and the like, provided that any two or more R^{iv} substituents may be linked to form a cycle, wherein the cycle may contain one or more annulated aromatic or aliphatic rings, one or more heteroatoms, or any combination thereof;

n3 is an integer selected from 1, 2, 3, 4, and 5; and

Some preferred examples of Q¹ therefore include the following:

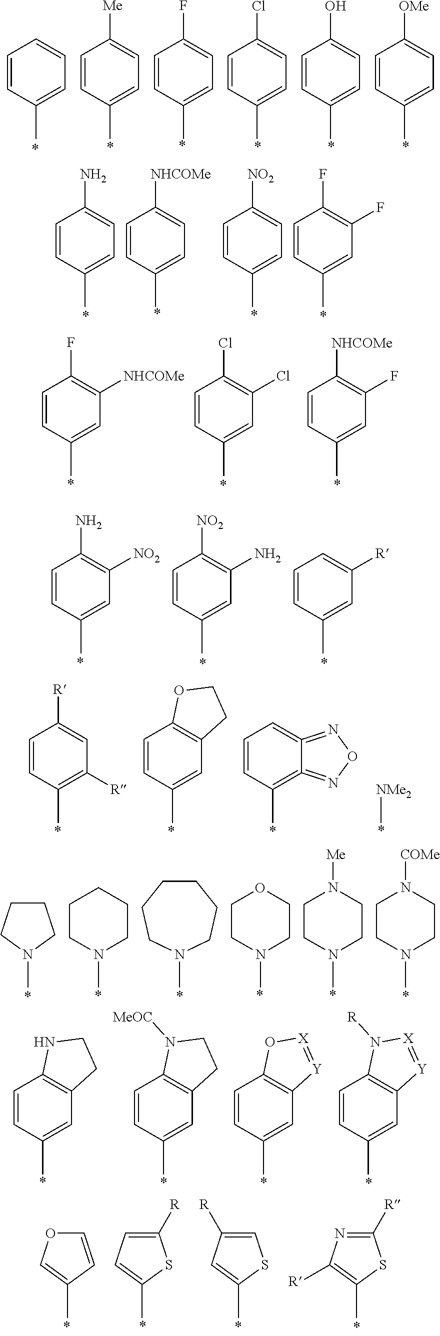

wherein:

the star represents the point of attachment to the remainder of the compound;

R, R′, and R″ are independently selected from H, alkyl, aryl, heteroaryl, halo, alkoxy, amido, acetamido, and the like;

n3 is an integer selected from 1 and 2; and

X and Y are independently selected from —CR— and —N—.

Also for example, Q² and Q³ are independently selected from substituted or unsubstituted C₅-C₃₀ aryl, substituted or unsubstituted C₅-C₃₀ heteroaryl, substituted or unsubstituted C₅-C₃₀ biaryl, substituted or unsubstituted C₅-C₃₀ heterobiaryl, and substituted or unsubstituted C₁-C₃₀ alkyl. In some preferred embodiments, Q² and Q³ are independently selected from biaryl and heterobiaryl moieties having a 6-member ring fused to a 5-membered ring, or a 6-member ring fused to another 6-member ring. Examples of Q² and Q³ include phenyl, 2-pyridyl, 3-pyridyl, 4-pyridyl, 5-pyridyl, 6-pyridyl, 2-thiazolyl, 3-thiazolyl, 4-thiazolyl, and 5-thiazolyl. For example, Q² and Q³ may independently have the structure

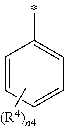

wherein n4 is selected from an integer in the range of 0-5, and each R⁴ is independently selected from hydrocarbyl and functional groups. In preferred embodiments, n4 is 0, 1, or 2, and each R⁴ is halo. For example, each R⁴ is independently fluoro or chloro. Some examples of preferred Q² and Q³ groups include phenyl rings with fluoro groups at the 2-, 3-, and/or 4-positions, chloro groups at the 2-, 3-, and/or 4-positions, or any combination thereof. One preferred example of Q³ is 3,4-dimethoxyphenyl, and one preferred example of Q² is phenyl. However, in some embodiments, Q³ is not 3,4-dimethoxyphenyl, and Q² is not phenyl. In some preferred embodiments, Q² is a pyridyl, thiophenyl, or thiazolyl substituent.

Also for example, R² is selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, C₆-C₃₀ aralkyl, and C₆-C₃₀ heteroatom-containing aralkyl, any of which may be substituted or unsubstituted. In some preferred embodiments, R² is lower alkyl or cycloalkyl. Some examples of R² include groups having the structure —CH₂—CH(R⁵)(R⁶), wherein R⁵ and R⁶ are independently selected from H, lower alkyl, aryl, and heteroaryl, or wherein R⁵ and R⁶ are taken together to form a 3- to 6-membered cycloalkyl ring, and wherein R⁵ and R⁶ may be further substituted. Other examples of R² include groups having the structure —CH₂—Ar, wherein Ar is an aryl or heteroaryl ring

Also for example, R³ is selected from H, alkyl, and —PO₃⁻²Mₓ, wherein M is an alkali or alkali-earth metal cation, and x has the value of 1 or 2 based on the charge of M. For example, M may be Li, Na, K, Rb, or Cs, in which cases x has the value of 2, or M may be Be, Mg, Ca, Sr, or Ba, in which cases x has the value of 1. In some embodiments, R³ is lower alkyl, such as lower linear, branched, or cyclic alkyl, an example of which is methyl. Alternatively, R³ and the oxygen to which it is attached (i.e., —OR³) may be any hydroxyl prodrug moiety—i.e., a moiety that is converted in vivo to a hydroxyl group. In one embodiment, hydroxyl prodrug moieties are esters that are metabolized in vivo by esterases or by other mechanisms to hydroxyl groups. Esters and other examples of prodrugs may be found in the relevant literature (see, e.g., Berge et al. (1977) “Pharmaceutical Salts”, J. Pharm. Sci. 66:1-19), and include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Specific examples of prodrug moieties are propionic acid ester and benzoic acid ester.

Also for example, R⁷ is substituted or unsubstituted lower alkyl. In some preferred embodiments, R⁷ is lower alkyl, branched lower alkyl, or cycloalkyl. For example, R⁷ may be selected from

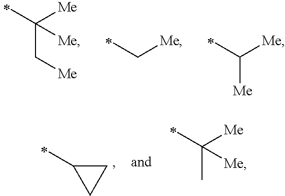

wherein the star represents the point of attachment to the remainder of the compound.

Also for example, L¹ is selected from substituted or unsubstituted C₁-C₁₂ alkylene, substituted or unsubstituted heteroatom-containing C₁-C₁₂ alkylene, an amino acid linking moiety, substituted or unsubstituted C₃-C₁₂ cycloalkylene, substituted or unsubstituted heteroatom-containing C₃-C₁₂ cycloalkylene, substituted or unsubstituted C₅-C₃₀ arylene, substituted or unsubstituted C₅-C₃₀ heteroarylene, substituted or unsubstituted C₆-C₃₀ aralkylene, substituted or unsubstituted heteroatom-containing C₅-C₃₀ aralkylene, substituted or unsubstituted C₆-C₃₀ alkarylene, and substituted or unsubstituted heteroatom-containing C₆-C₃₀ alkarylene. In one example, L¹ is —C(═O)—.

In some preferred embodiments, L¹ has the structure of formula (L^{1a})

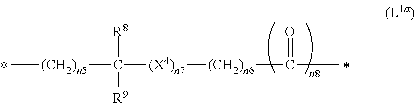

wherein:

the stars represent connection points to the remainder of the compound;

X⁴ is selected from —O— and —NR¹⁵—;

R⁸, and R⁹ are independently selected from H, substituted or unsubstituted lower alkyl, substituted or unsubstituted heteroatom-containing lower alkyl,

R¹⁵ is H or lower alkyl;

n7 and n8 are independently 0 or 1; and

n5 and n6 are independently selected from an integer in the range of 0-12, more preferably in the range of 1-6, most preferably 1, 2, or 3.

In some preferred embodiments, L¹ has the structure of —Ar¹—X⁵—, wherein Ar¹ is a substituted or unsubstituted 5- or 6-membered aromatic ring optionally containing one or more heteroatoms, and X⁵ is selected from a bond, —C(═O)—, —CH₂—, and —S(═O)₂—. For example, L¹ can have the structure of any one of formulae (L^{1b})-(L^{1h})

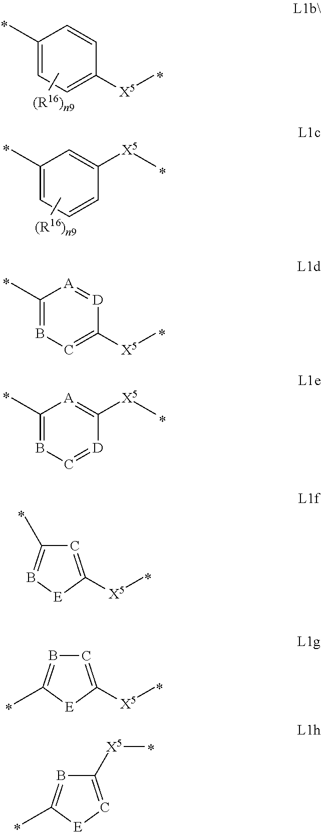

wherein:

the stars represent connection points to the remainder of the compound;

n9 is an integer in the range of 0-4;

each R¹⁶ is independently selected from H, lower alkyl, and functional groups;

A, B, C, and D are each independently selected from —CR′— and —N—; and

E is selected from —CR′₂—, —NR′—, and —O—. Examples of preferred Ar¹ groups include phenylene and pyridnylene.

In some preferred embodiments, L¹ has the structure of formula (L1i)

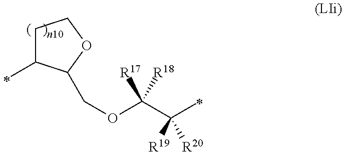

wherein:

the stars represent connection points to the remainder of the compound;

n10 is an integer selected from 1 and 2;

R¹⁷, R¹⁸, R¹⁹, and R²⁰ are independently selected from H and lower alkyl.

Also for example, L² is selected from substituted or unsubstituted lower alkylene. In preferred embodiments, L² is selected from —(CH₂)ₙ₁₁— and —(C═O)—(CH₂)ₙ₁₁—, wherein n11 is an integer in the range of 1-12, more preferably in the range of 1-6, most preferably 1 or 2.

For compounds having the structure of formula (Ia), examples of the moiety —X¹-L¹-X²-L²-X³— are provided in Table 1.

TABLE 1
Examples of —X¹—L¹—X²—L²—X³— for compounds having the structure of formula (Ia)

| X¹ | L¹ | X² | L² | X³ |
|---|---|---|---|---|
| — | —(CH₂)₂— | — | C═O | —NH— |
| — | —(CH₂)₃— | — | C═O | —NH— |
| — | C═O | —NH— | —(CH₂)₂— | —O— |
| — | —(CH₂)(C═O)— | —NH— | —(CH₂)₂— | —O— |
| — | —(CH₂)₂(C═O)— | —NH— | —(CH₂)₂— | —O— |
| — | —(CH₂)₃(C═O)— | —NH— | —(CH₂)₂— | —O— |
| — | —(CH₂)NH(C═O)— | —NH— | —(CH₂)₂— | —O— |
| — | 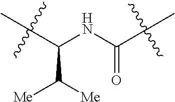 | —NH— | —(CH₂)₂— | —O— |
| — | 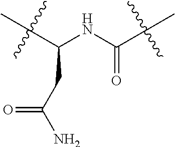 | —NH— | —(CH₂)₂— | —O— |
| —O— | —(CH₂)₁(C═O)— | —NH— | —(CH₂)₂— | —O— |
| —O— | —(CH₂)₂(C═O)— | —NH— | —(CH₂)₂— | —O— |
| —O— | —(CH₂)₃(C═O)— | —NH— | —(CH₂)₂— | —O— |
| —O— | 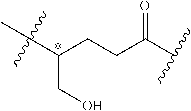 * R, S, or racemic | —NH— | —(CH₂)₂— | —O— |
| —O— | 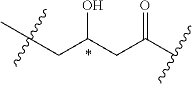 * R, S, or racemic | —NH— | —(CH₂)₂— | —O— |
| —O— | 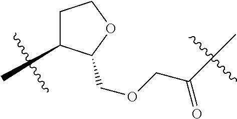 | —NH— | —(CH₂)₂— | —O— |
| —NH— | —(CH₂)₃— | —NH— | —(CH₂)₂— | —O— |
| —NMe— | —(CH₂)₂(C═O)— | —NH— | —(CH₂)₂— | —O— |
| — | —(CH₂)₂(C═O)— | —NH— | —(CH₂)₂— | —O— |
| — | 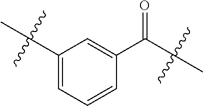 | —NH— | —(CH₂)₂— | —O— |
| — | 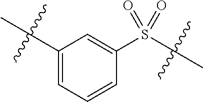 | —NH— | —(CH₂)₂— | —O— |
| — | 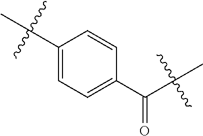 | —NH— | —(CH₂)₂— | —O— |
| — | 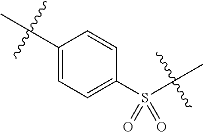 | —NH— | —(CH₂)₂— | —O— |
| — | 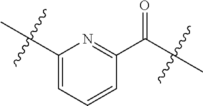 | —NH— | —(CH₂)₂— | —O— |
| — | 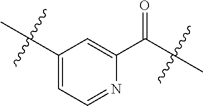 | —NH— | —(CH₂)₂— | —O— |
| — | 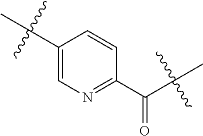 | —NH— | —(CH₂)₂— | —O— |
| — | 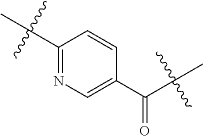 | —NH— | —(CH₂)₂— | —O— |
| —NH— | 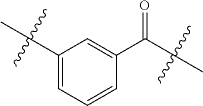 | —NH— | —(CH₂)₂— | —O— |
| —NH— | 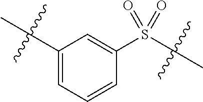 | —NH— | —(CH₂)₂— | —O— |
| —NH— | 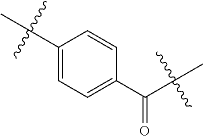 | —NH— | —(CH₂)₂— | —O— |
| —NH— | 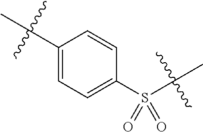 | —NH— | —(CH₂)₂— | —O— |
| —NH— | 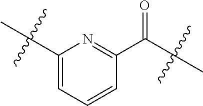 | —NH— | —(CH₂)₂— | —O— |
| —NH— | 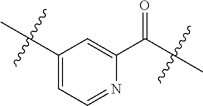 | —NH— | —(CH₂)₂— | —O— |
| —NH— | 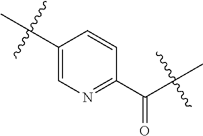 | —NH— | —(CH₂)₂— | —O— |
| —NH— | 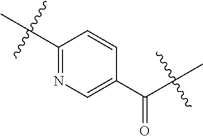 | —NH— | —(CH₂)₂— | —O— |
| — | —(CH₂)— | —NH— | —(C═O)(CH₂)— | —O— |
| — | 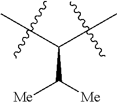 | —NH— | —(C═O)(CH₂)— | —O— |
| — | 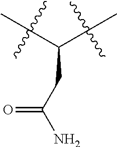 | —NH— | —(C═O)(CH₂)— | —O— |
| — | —(CH₂)₂— | —NH— | —(C═O)(CH₂)— | —O— |
| — | —(CH₂)₃— | —NH— | —(C═O)(CH₂)— | —O— |
| — | —(CH₂)₄— | —NH— | —(C═O)(CH₂)— | —O— |
| —O— | —(CH₂)₂— | —NH— | —(C═O)(CH₂)— | —O— |
| —O— | —(CH₂)₃— | —NH— | —(C═O)(CH₂)— | —O— |
| —O— | —(CH₂)₄— | —NH— | —(C═O)(CH₂)— | —O— |
| —O— | 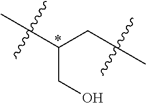 * R, S, or racemic | —NH— | —(C═O)(CH₂)— | —O— |
| —O— | 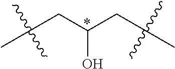 * R, S, or racemic | —NH— | —(C═O)(CH₂)— | —O— |
| —O— | 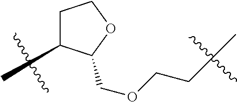 | —NH— | —(C═O)(CH₂)— | —O— |
| —NH— | —(CH₂)₃— | —NH— | —(C═O)(CH₂)— | —O— |
| —NH— | —(CH₂)₃— | —NH— | —(C═O)(CH₂)— | —O— |
| —NMe— | —(CH₂)₃— | —NH— | —(C═O)(CH₂)— | —O— |
| — | 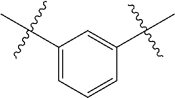 | —NH— | —(C═O)(CH₂)— | —O— |
| — | 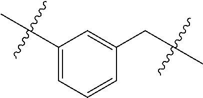 | —NH— | —(C═O)(CH₂)— | —O— |
| — | 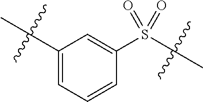 | —NH— | —(C═O)(CH₂)— | —O— |
| — | 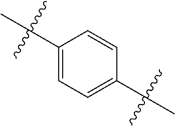 | —NH— | —(C═O)(CH₂)— | —O— |
| — | 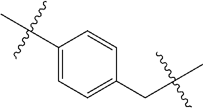 | —NH— | —(C═O)(CH₂)— | —O— |
| — | 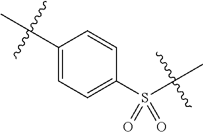 | —NH— | —(C═O)(CH₂)— | —O— |
| — | 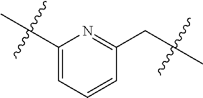 | —NH— | —(C═O)(CH₂)— | —O— |
| — | 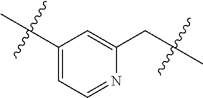 | —NH— | —(C═O)(CH₂)— | —O— |
| — | 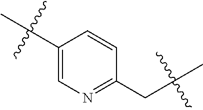 | —NH— | —(C═O)(CH₂)— | —O— |
| — | 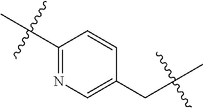 | —NH— | —(C═O)(CH₂)— | —O— |
| —NH— | 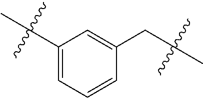 | —NH— | —(C═O)(CH₂)— | —O— |
| —NH— | 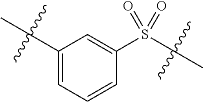 | —NH— | —(C═O)(CH₂)— | —O— |
| —NH— | 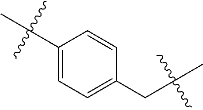 | —NH— | —(C═O)(CH₂)— | —O— |
| —NH— | 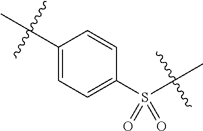 | —NH— | —(C═O)(CH₂)— | —O— |
| —NH— | 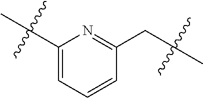 | —NH— | —(C═O)(CH₂)— | —O— |
| —NH— | 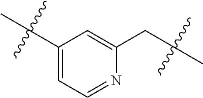 | —NH— | —(C═O)(CH₂)— | —O— |
| —NH— | 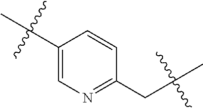 | —NH— | —(C═O)(CH₂)— | —O— |
| —NH— | 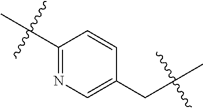 | —NH— | —(C═O)(CH₂)— | —O— |
| — | —(CH₂)ₙ— | — | C═O | —NR¹²— |
| — | —(CH₂)O(CH₂)— | — | C═O | —NR¹²— |
| — | —(CH₂)NR¹(CH₂)— | — | C═O | —NR¹²— |
| — | —(CR³R⁴)(C═O)— | —NR¹¹— | —(CH₂)₂— | —O— |
| — | —(CH₂)ₙ(C═O)— | —NR¹¹— | —(CH₂)₂— | —O— |
| — | —(CH₂)O(CH₂)(C═O)— | —NR¹¹— | C═O | —O— |
| — | —(CH₂)NR¹(CH)₂(C═O)— | —NR¹¹— | C═O | —O— |
| — | —(CH₂)NR¹(C═O)— | —NR¹¹— | —(CH₂)₂— | —O— |
| — | 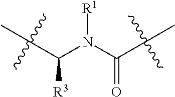 | —NR¹¹— | —(CH₂)₂— | —O— |
| —O— | —(CH₂)₁(C═O)— | —NR¹¹— | —(CH₂)₂— | —O— |
| —NR¹⁰— | —(CH₂)₂(C═O)— | —NR¹¹— | —(CH₂)₂— | —O— |
| — | 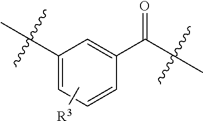 | —NR¹¹— | —(CH₂)₂— | —O— |
| — | 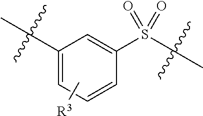 | —NR¹¹— | —(CH₂)₂— | —O— |
| — | 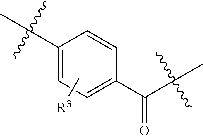 | —NR¹¹— | —(CH₂)₂— | —O— |
| — | 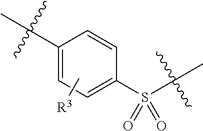 | —NR¹¹— | —(CH₂)₂— | —O— |
| — | 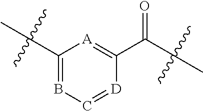 | —NR¹¹— | —(CH₂)₂— | —O— |
| — | 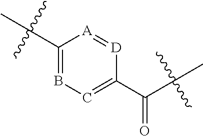 | —NR¹¹— | —(CH₂)₂— | —O— |
| — | 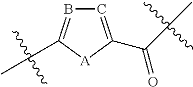 | —NR¹¹— | —(CH₂)₂— | —O— |
| — | 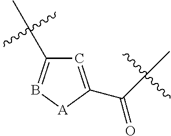 | —NR¹¹— | —(CH₂)₂— | —O— |
| — | 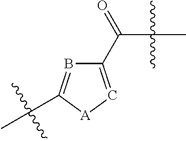 | —NR¹¹— | —(CH₂)₂— | —O— |
| —NR¹⁰— | 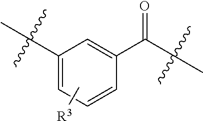 | —NR¹¹— | —(CH₂)₂— | —O— |
| —NR¹⁰— | 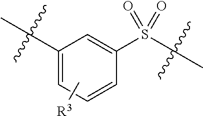 | —NR¹¹— | —(CH₂)₂— | —O— |
| —NR¹⁰— | 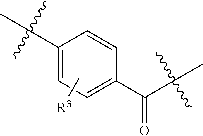 | —NR¹¹— | —(CH₂)₂— | —O— |
| —NR¹⁰— | 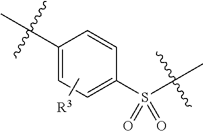 | —NR¹¹— | —(CH₂)₂— | —O— |
| —NR¹⁰— | 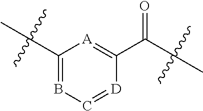 | —NR¹¹— | —(CH₂)₂— | —O— |
| —NR¹⁰— | 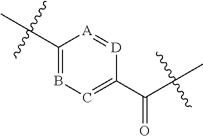 | —NR¹¹— | —(CH₂)₂— | —O— |
| —NR¹⁰— | 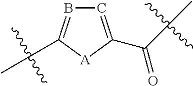 | —NR¹¹— | —(CH₂)₂— | —O— |
| —NR¹⁰— | 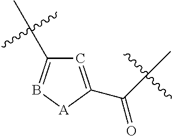 | —NR¹¹— | —(CH₂)₂— | —O— |
| —NR¹⁰— | 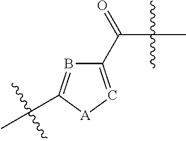 | —NR¹¹— | —(CH₂)₂— | —O— |
| — | —(CH₂)ₙ— | —NR¹¹— | —(C═O)(CH₂)— | —O— |
| — | —(CR³R⁴)(C═O)— | —NR¹¹— | —(C═O)(CH₂)— | —O— |
| —O— | —(CH₂)ₙ— | —NR¹¹— | —(C═O)(CH₂)— | —O— |
| | n >= 2 | | | |
| —O— | 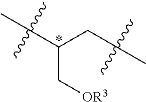 * R, S, or racemic | —NR¹¹— | —(C═O)(CH₂)— | —O— |
| —O— | 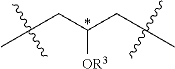 * R, S, or racemic | —NR¹¹— | —(C═O)(CH₂)— | —O— |
| —O— | 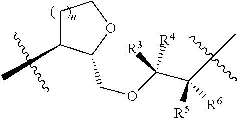 | —NR¹¹— | —(C═O)(CH₂)— | —O— |
| —NR¹⁰— | —(CH₂)₃— | —NR¹¹— | —(C═O)(CH₂)— | —O— |
| — | 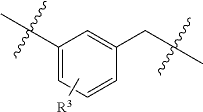 | —NR¹¹— | —(C═O)(CH₂)— | —O— |
| — | 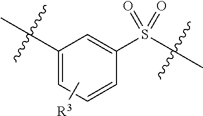 | —NR¹¹— | —(C═O)(CH₂)— | —O— |
| — | 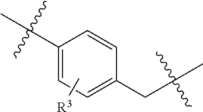 | —NR¹¹— | —(C═O)(CH₂)— | —O— |
| — | 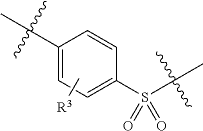 | —NR¹¹— | —(C═O)(CH₂)— | —O— |
| — | 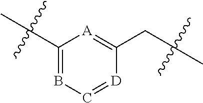 | —NR¹¹— | —(C═O)(CH₂)— | —O— |
| — | 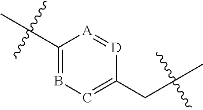 | —NR¹¹— | —(C═O)(CH₂)— | —O— |
| — | 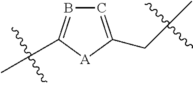 | —NR¹¹— | —(C═O)(CH₂)— | —O— |
| — | 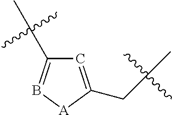 | —NR¹¹— | —(C═O)(CH₂)— | —O— |
| — | 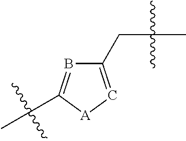 | —NR¹¹— | —(C═O)(CH₂)— | —O— |
| —NR¹⁰— | 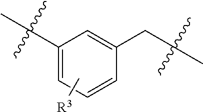 | —NR¹¹— | —(C═O)(CH₂)— | —O— |
| —NR¹⁰— | 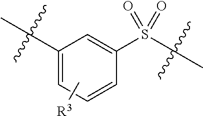 | —NR¹¹— | —(C═O)(CH₂)— | —O— |
| —NR¹⁰— | 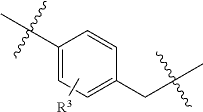 | —NR¹¹— | —(C═O)(CH₂)— | —O— |
| —NR¹⁰— | 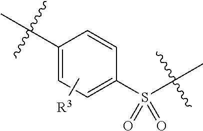 | —NR¹¹— | —(C═O)(CH₂)— | —O— |
| —NR¹⁰— | 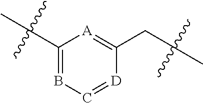 | —NR¹¹— | —(C═O)(CH₂)— | —O— |
| —NR¹⁰— | 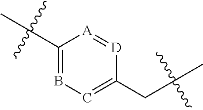 | —NR¹¹— | —(C═O)(CH₂)— | —O— |
| —NR¹⁰— | 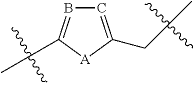 | —NR¹¹— | —(C═O)(CH₂)— | —O— |
| —NR¹⁰— | 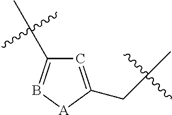 | —NR¹¹— | —(C═O)(CH₂)— | —O— |
| —NR¹⁰— | 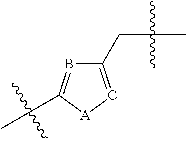 | —NR¹¹— | —(C═O)(CH₂)— | —O— |

Also for example, L³ is selected from lower alkylene, arylene, biaryl, heteroarylene, and heterobiarylene, any of which may be substituted or unsubstituted. In some embodiments, L³ has the structure

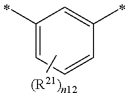

wherein n12 is an integer in the range of 0-4, and wherein each R²¹ is independently selected from H, halo, and hydrocarbyl.

In some preferred embodiments, —X¹-L¹-X²-L²-X³-L³- is other than —(CH₂)₂—C(═O)—NH—C₆H₄—. In some embodiments, —X¹-L¹-X²-L²-X³— is other than —(CH₂)_{b1}—(OCH₂CH₂)_{b2}—(CH₂)_{b3}—, wherein b1 and b3 are each integers between 0 and 4 and b2 is an integer between 0 and 2. For example, —X¹-L¹-X²-L²-X³— is other than —(CH₂)₂—, or other than —(CH₂)₈—, or other than —(CH₂)₂—(OCH₂CH₂)—, or other than —(CH₂)₂—(OCH₂CH₂)₂—.

In some embodiments, the compounds of formula (A) have the structure of formula (IIa)

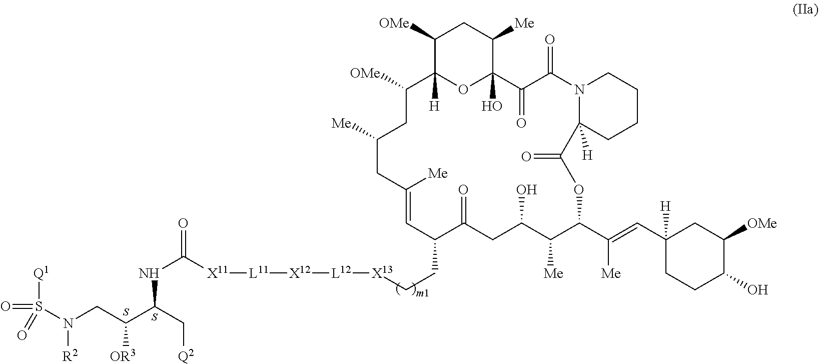

wherein:

Q¹, Q², R², and R³ are as defined previously;

m1 is an integer selected from 1 and 2;

X¹¹, L¹¹, and X¹² are as defined for X¹, L¹, and X², respectively;

L¹² is selected from alkylene and alkenylene; and

X¹³ is selected from a bond and —O—.

For example, L¹² is selected from substituted or unsubstituted lower alkylene and substituted or substituted lower alkenylene. In preferred embodiments, L¹² is selected from —(CH₂)ₙ₈—, —CH₂—CH═CH—, and —(C═O)—(CH₂)ₙ₈—, wherein n8 is an integer in the range of 1-12, more preferably in the range of 1-6, most preferably 1 or 2.

For compounds having the structure of formula (IIa), examples of the moiety —X¹¹-L¹¹-X¹²-L¹²-X¹³—(CH₂)ₘ₁— are provided in Table 2.

TABLE 2
Examples of —X¹¹—L¹¹—X¹²—L¹²—X¹³—(CH₂)ₘ₁— for compounds having the structure of formula (IIa)

| X¹¹ | L¹¹ | X¹² | L¹² | X¹³ | m1 |
|---|---|---|---|---|---|
| — | C═O | —NH— | —(CH₂)₂— | —O— | 1,2 |
| — | —(CH₂)(C═O)— | —NH— | —(CH₂)₂— | —O— | 1,2 |
| — | —(CH₂)₂(C═O)— | —NH— | —(CH₂)₂— | —O— | 1,2 |
| — | —(CH₂)₃(C═O)— | —NH— | —(CH₂)₂— | —O— | 1,2 |
| — | —(CH₂)NH(C═O)— | —NH— | —(CH₂)₂— | —O— | 1,2 |
| — | 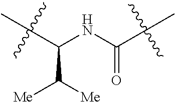 | —NH— | —(CH₂)₂— | —O— | 1,2 |
| — | 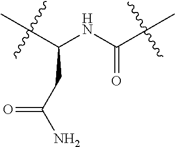 | —NH— | —(CH₂)₂— | —O— | 1,2 |
| —O— | —(CH₂)₁(C═O)— | —NH— | —(CH₂)₂— | —O— | 1,2 |
| —O— | —(CH₂)₂(C═O)— | —NH— | —(CH₂)₂— | —O— | 1,2 |
| —O— | —(CH₂)₃(C═O)— | —NH— | —(CH₂)₂— | —O— | 1,2 |
| —O— | 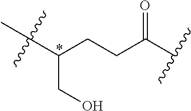 * R, S, or racemic | —NH— | —(CH₂)₂— | —O— | 1,2 |
| —O— | 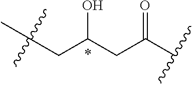 * R, S, or racemic | —NH— | —(CH₂)₂— | —O— | 1,2 |
| —O— | 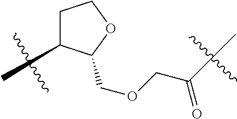 | —NH— | —(CH₂)₂— | —O— | 1,2 |
| —O— | —(CH₂)₃— | —NH— | —(CH₂)₂— | —O— | 1,2 |
| —NH— | —(CH₂)₂(C═O)— | —NH— | —(CH₂)₂— | —O— | 1,2 |
| —NMe— | —(CH₂)₂(C═O)— | —NH— | —(CH₂)₂— | —O— | 1,2 |
| — | 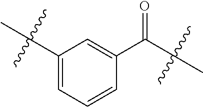 | —NH— | —(CH₂)₂— | —O— | 1,2 |
| — | 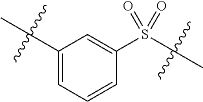 | —NH— | —(CH₂)₂— | —O— | 1,2 |
| — | 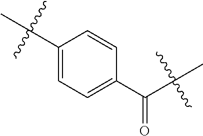 | —NH— | —(CH₂)₂— | —O— | 1,2 |
| — | 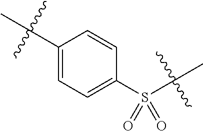 | —NH— | —(CH₂)₂— | —O— | 1,2 |
| — | 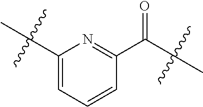 | —NH— | —(CH₂)₂— | —O— | 1,2 |
| — | 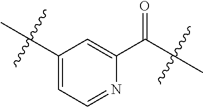 | —NH— | —(CH₂)₂— | —O— | 1,2 |
| — | 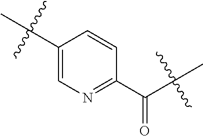 | —NH— | —(CH₂)₂— | —O— | 1,2 |
| — | 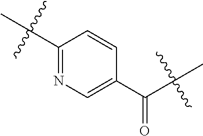 | —NH— | —(CH₂)₂— | —O— | 1,2 |
| —NH— | 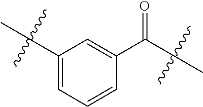 | —NH— | —(CH₂)₂— | —O— | 1,2 |
| —NH— | 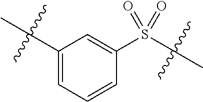 | —NH— | —(CH₂)₂— | —O— | 1,2 |
| —NH— | 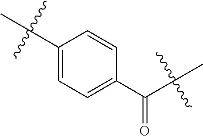 | —NH— | —(CH₂)₂— | —O— | 1,2 |
| —NH— | 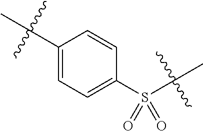 | —NH— | —(CH₂)₂— | —O— | 1,2 |
| —NH— | 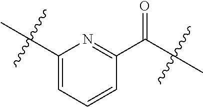 | —NH— | —(CH₂)₂— | —O— | 1,2 |
| —NH— | 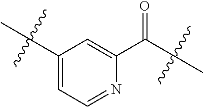 | —NH— | —(CH₂)₂— | —O— | 1,2 |
| —NH— | 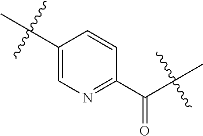 | —NH— | —(CH₂)₂— | —O— | 1,2 |
| —NH— | 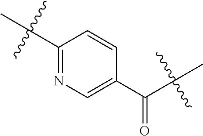 | —NH— | —(CH₂)₂— | —O— | 1,2 |
| — | —(CH₂)— | —NH— | —(C═O)(CH₂)— | —O— | 1,2 |
| — | 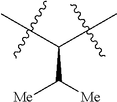 | —NH— | —(C═O)(CH₂)— | —O— | 1,2 |
| — | 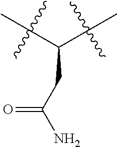 | —NH— | —(C═O)(CH₂)— | —O— | 1,2 |
| — | —(CH₂)₂— | —NH— | —(C═O)(CH₂)— | —O— | 1,2 |
| — | —(CH₂)₃— | —NH— | —(C═O)(CH₂)— | —O— | 1,2 |
| — | —(CH₂)₄— | —NH— | —(C═O)(CH₂)— | —O— | 1,2 |
| —O— | —(CH₂)₂— | —NH— | —(C═O)(CH₂)— | —O— | 1,2 |
| —O— | —(CH₂)₃— | —NH— | —(C═O)(CH₂)— | —O— | 1,2 |
| —O— | —(CH₂)₄— | —NH— | —(C═O)(CH₂)— | —O— | 1,2 |
| —O— | 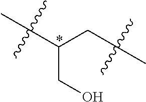 * R, S, or racemic | —NH— | —(C═O)(CH₂)— | —O— | 1,2 |
| —O— | 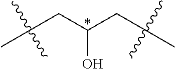 * R, S, or racemic | —NH— | —(C═O)(CH₂)— | —O— | 1,2 |
| —O— | 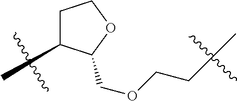 | —NH— | —(C═O)(CH₂)— | —O— | 1,2 |
| —NH— | —(CH₂)₃— | —NH— | —(C═O)(CH₂)— | —O— | 1,2 |
| —NH— | —(CH₂)₃— | —NH— | —(C═O)(CH₂)— | —O— | 1,2 |
| —NMe— | —(CH₂)₃— | —NH— | —(C═O)(CH₂)— | —O— | 1,2 |
| — | 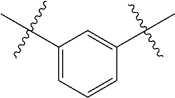 | —NH— | —(C═O)(CH₂)— | —O— | 1,2 |
| — | 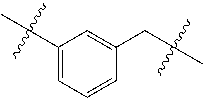 | —NH— | —(C═O)(CH₂)— | —O— | 1,2 |
| — | 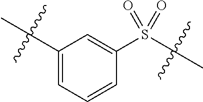 | —NH— | —(C═O)(CH₂)— | —O— | 1,2 |
| — | 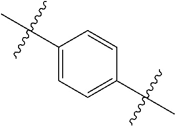 | —NH— | —(C═O)(CH₂)— | —O— | 1,2 |
| — | 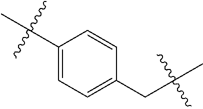 | —NH— | —(C═O)(CH₂)— | —O— | 1,2 |
| — | 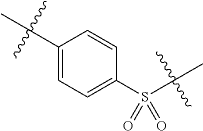 | —NH— | —(C═O)(CH₂)— | —O— | 1,2 |
| — | 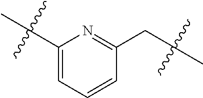 | —NH— | —(C═O)(CH₂)— | —O— | 1,2 |
| — | 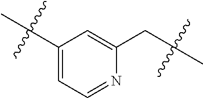 | —NH— | —(C═O)(CH₂)— | —O— | 1,2 |
| — | 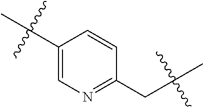 | —NH— | —(C═O)(CH₂)— | —O— | 1,2 |
| — | 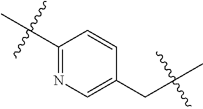 | —NH— | —(C═O)(CH₂)— | —O— | 1,2 |
| —NH— | 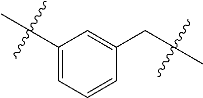 | —NH— | —(C═O)(CH₂)— | —O— | 1,2 |
| —NH— | 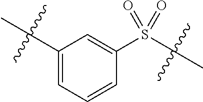 | —NH— | —(C═O)(CH₂)— | —O— | 1,2 |
| —NH— | 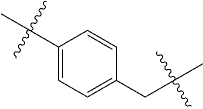 | —NH— | —(C═O)(CH₂)— | —O— | 1,2 |
| —NH— | 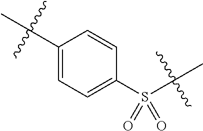 | —NH— | —(C═O)(CH₂)— | —O— | 1,2 |
| —NH— | 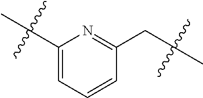 | —NH— | —(C═O)(CH₂)— | —O— | 1,2 |
| —NH— | 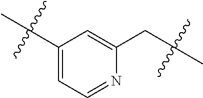 | —NH— | —(C═O)(CH₂)— | —O— | 1,2 |
| —NH— | 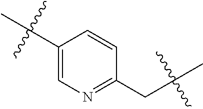 | —NH— | —(C═O)(CH₂)— | —O— | 1,2 |
| —NH— | 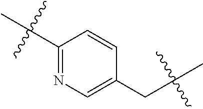 | —NH— | —(C═O)(CH₂)— | —O— | 1,2 |
| — | —(CR³R⁴)(C═O)— | —NR¹¹— | —(CH₂)₂— | —O— | 1,2 |
| — | —(CH₂)ₙ(C═O)— | —NR¹¹— | —(CH₂)₂— | —O— | 1,2 |
| — | —(CH₂)O(CH₂)(C═O)— | —NR¹¹— | C═O | —O— | 1,2 |
| — | —(CH₂)NR¹(CH)₂(C═O)— | —NR¹¹— | C═O | —O— | 1,2 |
| — | —(CH₂)NR¹(C═O)— | —NR¹¹— | —(CH₂)₂— | —O— | 1,2 |
| — | 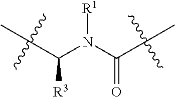 | —NR¹¹— | —(CH₂)₂— | —O— | 1,2 |
| —O— | —(CH₂)₁(C═O)— | —NR¹¹— | —(CH₂)₂— | —O— | 1,2 |
| —NR¹⁰— | —(CH₂)₂(C═O)— | —NR¹¹— | —(CH₂)₂— | —O— | 1,2 |
| — | 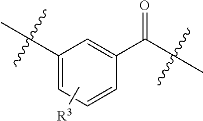 | —NR¹¹— | —(CH₂)₂— | —O— | 1,2 |
| — | 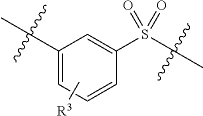 | —NR¹¹— | —(CH₂)₂— | —O— | 1,2 |
| — | 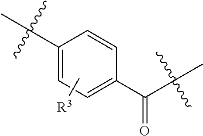 | —NR¹¹— | —(CH₂)₂— | —O— | 1,2 |
| — | 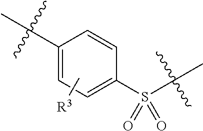 | —NR¹¹— | —(CH₂)₂— | —O— | 1,2 |
| — | 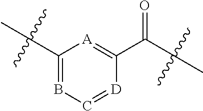 | —NR¹¹— | —(CH₂)₂— | —O— | 1,2 |
| — | 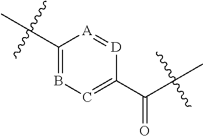 | —NR¹¹— | —(CH₂)₂— | —O— | 1,2 |
| — | 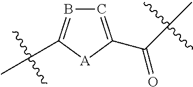 | —NR¹¹— | —(CH₂)₂— | —O— | 1,2 |
| — | 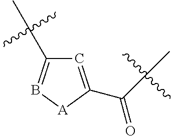 | —NR¹¹— | —(CH₂)₂— | —O— | 1,2 |
| — | 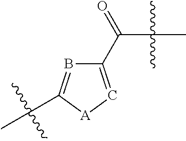 | —NR¹¹— | —(CH₂)₂— | —O— | 1,2 |
| —NR¹⁰— | 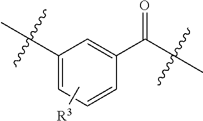 | —NR¹¹— | —(CH₂)₂— | —O— | 1,2 |
| —NR¹⁰— | 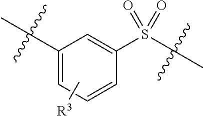 | —NR¹¹— | —(CH₂)₂— | —O— | 1,2 |
| —NR¹⁰— | 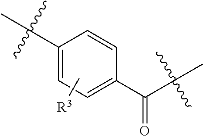 | —NR¹¹— | —(CH₂)₂— | —O— | 1,2 |
| —NR¹⁰— | 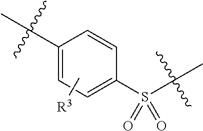 | —NR¹¹— | —(CH₂)₂— | —O— | 1,2 |
| —NR¹⁰— | 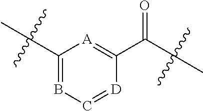 | —NR¹¹— | —(CH₂)₂— | —O— | 1,2 |
| —NR¹⁰— | 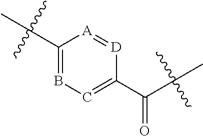 | —NR¹¹— | —(CH₂)₂— | —O— | 1,2 |
| —NR¹⁰— | 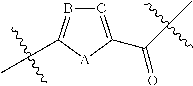 | —NR¹¹— | —(CH₂)₂— | —O— | 1,2 |
| —NR¹⁰— | 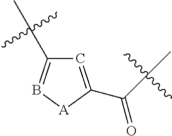 | —NR¹¹— | —(CH₂)₂— | —O— | 1,2 |
| —NR¹⁰— | 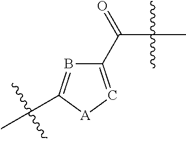 | —NR¹¹— | —(CH₂)₂— | —O— | 1,2 |
| — | —(CH₂)ₙ— | —NR¹¹— | —(C═O)(CH₂)— | —O— | 1,2 |
| — | —(CR³R⁴)(C═O)— | —NR¹¹— | —(C═O)(CH₂)— | —O— | 1,2 |
| —O— | —(CH₂)ₙ— | —NR¹¹— | —(C═O)(CH₂)— | —O— | 1,2 |
| | n >= 2 | | | | |
| —O— | 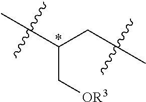 *R, S, or racemic | —NR¹¹— | —(C═O)(CH₂)— | —O— | 1,2 |
| —O— | 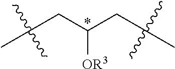 * R, S, or racemic | —NR¹¹— | —(C═O)(CH₂)— | —O— | 1,2 |
| —O— | 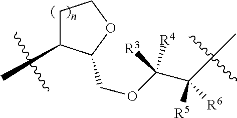 | —NR¹¹— | —(C═O)(CH₂)— | —O— | 1,2 |
| —NR¹⁰— | —(CH₂)₃— | —NR¹¹— | —(C═O)(CH₂)— | —O— | 1,2 |
| — | 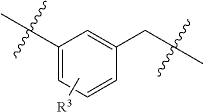 | —NR¹¹— | —(C═O)(CH₂)— | —O— | 1,2 |
| — | 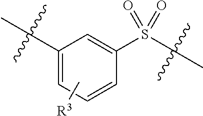 | —NR¹¹— | —(C═O)(CH₂)— | —O— | 1,2 |
| — | 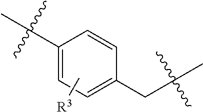 | —NR¹¹— | —(C═O)(CH₂)— | —O— | 1,2 |
| — | 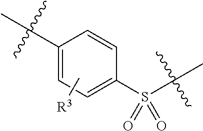 | —NR¹¹— | —(C═O)(CH₂)— | —O— | 1,2 |
| — | 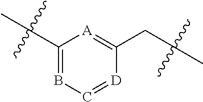 | —NR¹¹— | —(C═O)(CH₂)— | —O— | 1,2 |
| — | 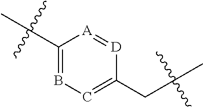 | —NR¹¹— | —(C═O)(CH₂)— | —O— | 1,2 |
| — | 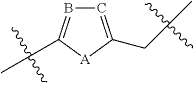 | —NR¹¹— | —(C═O)(CH₂)— | —O— | 1,2 |
| — | 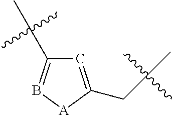 | —NR¹¹— | —(C═O)(CH₂)— | —O— | 1,2 |
| — | 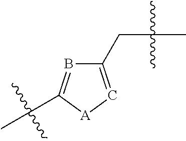 | —NR¹¹— | —(C═O)(CH₂)— | —O— | 1,2 |
| —NR¹⁰— | 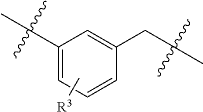 | —NR¹¹— | —(C═O)(CH₂)— | —O— | 1,2 |
| —NR¹⁰— | 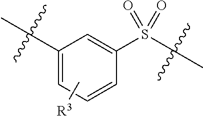 | —NR¹¹— | —(C═O)(CH₂)— | —O— | 1,2 |
| —NR¹⁰— | 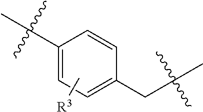 | —NR¹¹— | —(C═O)(CH₂)— | —O— | 1,2 |
| —NR¹⁰— | 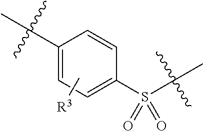 | —NR¹¹— | —(C═O)(CH₂)— | —O— | 1,2 |
| —NR¹⁰— | 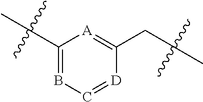 | —NR¹¹— | —(C═O)(CH₂)— | —O— | 1,2 |
| —NR¹⁰— | 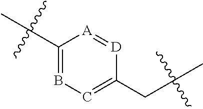 | —NR¹¹— | —(C═O)(CH₂)— | —O— | 1,2 |
| —NR¹⁰— | 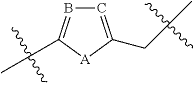 | —NR¹¹— | —(C═O)(CH₂)— | —O— | 1,2 |
| —NR¹⁰— | 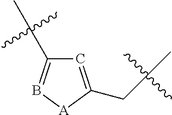 | —NR¹¹— | —(C═O)(CH₂)— | —O— | 1,2 |
| —NR¹⁰— | 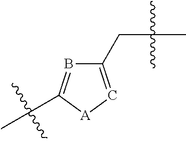 | —NR¹¹— | —(C═O)(CH₂)— | —O— | 1,2 |
| — | C═O | —NH— | CH₂(CH═CH) | — | 1 |
| — | —(CH₂)(C═O)— | —NH— | CH₂(CH═CH) | — | 1 |
| — | —(CH₂)₂(C═O)— | —NH— | CH₂(CH═CH) | — | 1 |
| — | —(CH₂)₃(C═O)— | —NH— | CH₂(CH═CH) | — | 1 |
| — | —(CH₂)NH(C═O)— | —NH— | CH₂(CH═CH) | — | 1 |
| — | 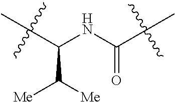 | —NH— | CH₂(CH═CH) | — | 1 |
| — | 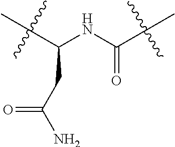 | —NH— | CH₂(CH═CH) | — | 1 |
| —O— | —(CH₂)₁(C═O)— | —NH— | CH₂(CH═CH) | — | 1 |
| —O— | —(CH₂)₂(C═O)— | —NH— | CH₂(CH═CH) | — | 1 |
| —O— | —(CH₂)₃(C═O)— | —NH— | CH₂(CH═CH) | — | 1 |
| —O— | 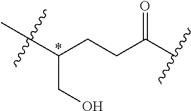 * R, S, or racemic | —NH— | CH₂(CH═CH) | — | 1 |
| —O— | 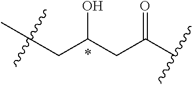 * R, S, or racemic | —NH— | CH₂(CH═CH) | — | 1 |
| —O— | 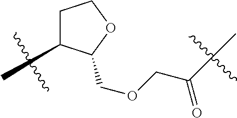 | —NH— | CH₂(CH═CH) | — | 1 |
| —O— | —(CH₂)₃— | —NH— | CH₂(CH═CH) | — | 1 |
| —NH— | —(CH₂)₂(C═O)— | —NH— | CH₂(CH═CH) | — | 1 |
| —NMe— | —(CH₂)₂(C═O)— | —NH— | CH₂(CH═CH) | — | 1 |
| — | 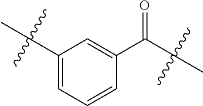 | —NH— | CH₂(CH═CH) | — | 1 |
| — | 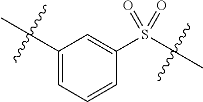 | —NH— | CH₂(CH═CH) | — | 1 |
| — | 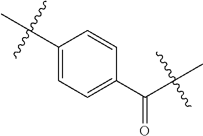 | —NH— | CH₂(CH═CH) | — | 1 |
| — | 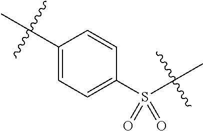 | —NH— | CH₂(CH═CH) | — | 1 |
| — | 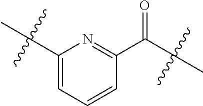 | —NH— | CH₂(CH═CH) | — | 1 |
| — | 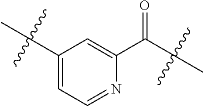 | —NH— | CH₂(CH═CH) | — | 1 |
| — | 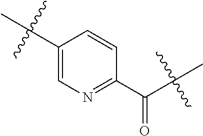 | —NH— | CH₂(CH═CH) | — | 1 |
| — | 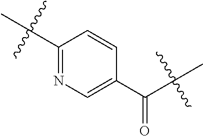 | —NH— | CH₂(CH═CH) | — | 1 |
| —NH— | 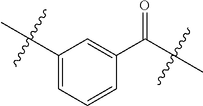 | —NH— | CH₂(CH═CH) | — | 1 |
| —NH— | 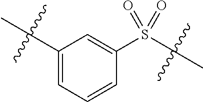 | —NH— | CH₂(CH═CH) | — | 1 |
| —NH— | 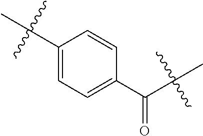 | —NH— | CH₂(CH═CH) | — | 1 |
| —NH— | 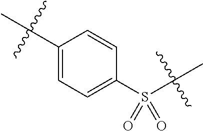 | —NH— | CH₂(CH═CH) | — | 1 |
| —NH— | 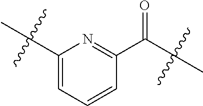 | —NH— | CH₂(CH═CH) | — | 1 |
| —NH— | 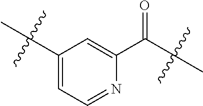 | —NH— | CH₂(CH═CH) | — | 1 |
| —NH— | 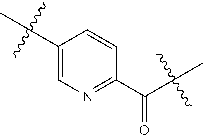 | —NH— | CH₂(CH═CH) | — | 1 |
| —NH— | 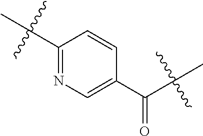 | —NH— | CH₂(CH═CH) | — | 1 |
| — | —(CH₂)— | —NH— | —(C═O)(CH═CH)— | — | 1 |
| — | 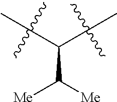 | —NH— | —(C═O)(CH═CH)— | — | 1 |
| — | 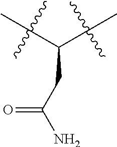 | —NH— | —(C═O)(CH═CH)— | — | 1 |
| — | —(CH₂)₂— | —NH— | —(C═O)(CH═CH)— | — | 1 |
| — | —(CH₂)₃— | —NH— | —(C═O)(CH═CH)— | — | 1 |
| — | —(CH₂)₄— | —NH— | —(C═O)(CH═CH)— | — | 1 |
| —O— | —(CH₂)₂— | —NH— | —(C═O)(CH═CH)— | — | 1 |
| —O— | —(CH₂)₃— | —NH— | —(C═O)(CH═CH)— | — | 1 |
| —O— | —(CH₂)₄— | —NH— | —(C═O)(CH═CH)— | — | 1 |
| —O— | 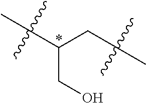 * R, S, or racemic | —NH— | —(C═O)(CH═CH)— | — | 1 |
| —O— | 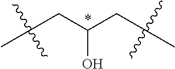 * R, S, or racemic | —NH— | —(C═O)(CH═CH)— | — | 1 |
| —O— | 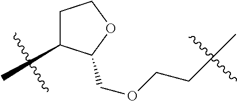 | —NH— | —(C═O)(CH═CH)— | — | 1 |
| —NH— | —(CH₂)₃— | —NH— | —(C═O)(CH═CH)— | — | 1 |
| —NH— | —(CH₂)₃— | —NH— | —(C═O)(CH═CH)— | — | 1 |
| —NMe— | —(CH₂)₃— | —NH— | —(C═O)(CH═CH)— | — | 1 |
| — | 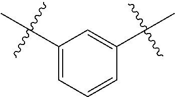 | —NH— | —(C═O)(CH═CH)— | — | 1 |
| — | 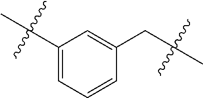 | —NH— | —(C═O)(CH═CH)— | — | 1 |
| — | 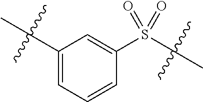 | —NH— | —(C═O)(CH═CH)— | — | 1 |
| — | 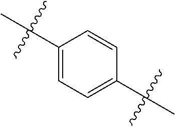 | —NH— | —(C═O)(CH═CH)— | — | 1 |
| — | 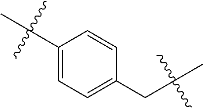 | —NH— | —(C═O)(CH═CH)— | — | 1 |
| — | 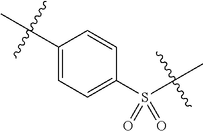 | —NH— | —(C═O)(CH═CH)— | — | 1 |
| — | 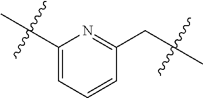 | —NH— | —(C═O)(CH═CH)— | — | 1 |
| — | 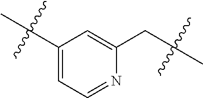 | —NH— | —(C═O)(CH═CH)— | — | 1 |
| — | 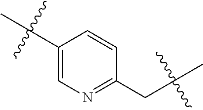 | —NH— | —(C═O)(CH═CH)— | — | 1 |
| — | 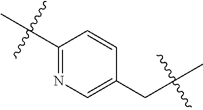 | —NH— | —(C═O)(CH═CH)— | — | 1 |
| —NH— | 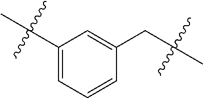 | —NH— | —(C═O)(CH═CH)— | — | 1 |
| —NH— | 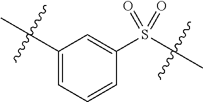 | —NH— | —(C═O)(CH═CH)— | — | 1 |
| —NH— | 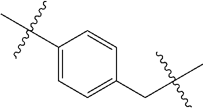 | —NH— | —(C═O)(CH═CH)— | — | 1 |
| —NH— | 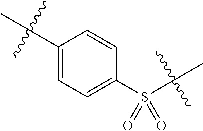 | —NH— | —(C═O)(CH═CH)— | — | 1 |
| —NH— | 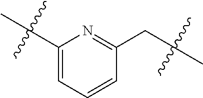 | —NH— | —(C═O)(CH═CH)— | — | 1 |
| —NH— | 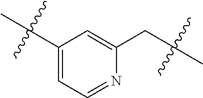 | —NH— | —(C═O)(CH═CH)— | — | 1 |
| —NH— | 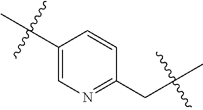 | —NH— | —(C═O)(CH═CH)— | — | 1 |
| —NH— | 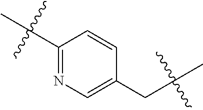 | —NH— | —(C═O)(CH═CH)— | — | 1 |
| — | —(CR³R⁴)(C═O)— | —NR¹¹— | CH₂(CH═CH) | — | 1 |
| — | —(CH₂)ₙ(C═O)— | —NR¹¹— | CH₂(CH═CH) | — | 1 |
| — | —(CH₂)O(CH₂)(C═O)— | —NR¹¹— | CH₂(CH═CH) | — | 1 |
| — | —(CH₂)NR¹(CH)₂(C═O)— | —NR¹¹— | CH₂(CH═CH) | — | 1 |
| — | —(CH₂)NR¹(C═O)— | —NR¹¹— | CH₂(CH═CH) | — | 1 |
| — | 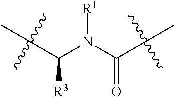 | —NR¹¹— | CH₂(CH═CH) | — | 1 |
| —O— | —(CH₂)₁(C═O)— | —NR¹¹— | CH₂(CH═CH) | — | 1 |
| —NR¹⁰— | —(CH₂)₂(C═O)— | —NR¹¹— | CH₂(CH═CH) | — | 1 |
| — | 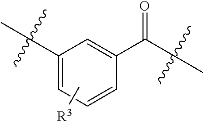 | —NR¹¹— | CH₂(CH═CH) | — | 1 |
| — | 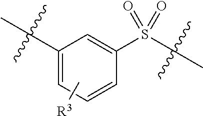 | —NR¹¹— | CH₂(CH═CH) | — | 1 |
| — | 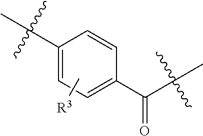 | —NR¹¹— | CH₂(CH═CH) | — | 1 |
| — | 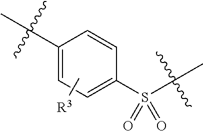 | —NR¹¹— | CH₂(CH═CH) | — | 1 |
| — | 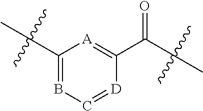 | —NR¹¹— | CH₂(CH═CH) | — | 1 |
| — | 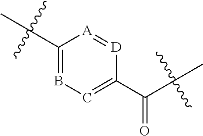 . | —NR¹¹— | CH₂(CH═CH) | — | 1 |
| — | 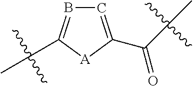 | —NR¹¹— | CH₂(CH═CH) | — | 1 |
| — | 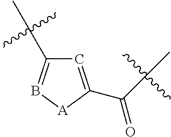 | —NR¹¹— | CH₂(CH═CH) | — | 1 |
| — | 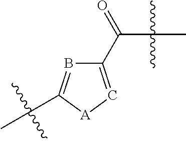 | —NR¹¹— | CH₂(CH═CH) | — | 1 |
| —NR¹⁰— | 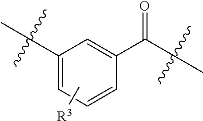 | —NR¹¹— | CH₂(CH═CH) | — | 1 |
| —NR¹⁰— | 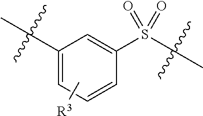 | —NR¹¹— | CH₂(CH═CH) | — | 1 |
| —NR¹⁰— | 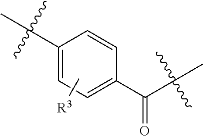 | —NR¹¹— | CH₂(CH═CH) | — | 1 |
| —NR¹⁰— | 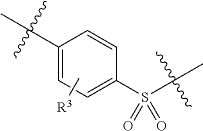 | —NR¹¹— | CH₂(CH═CH) | — | 1 |
| —NR¹⁰— | 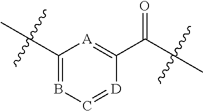 | —NR¹¹— | CH₂(CH═CH) | — | 1 |
| —NR¹⁰— | 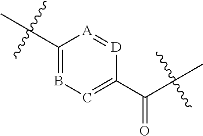 | —NR¹¹— | CH₂(CH═CH) | — | 1 |
| —NR¹⁰— | 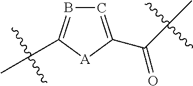 | —NR¹¹— | CH₂(CH═CH) | — | 1 |
| —NR¹⁰— | 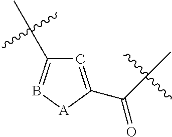 | —NR¹¹— | CH₂(CH═CH) | — | 1 |
| —NR¹⁰— | 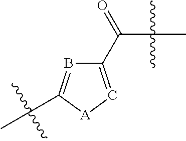 | —NR¹¹— | CH₂(CH═CH) | — | 1 |
| — | —(CH₂)ₙ— | —NR¹¹— | —(C═O)(CH═CH)— | — | 1 |
| — | —(CR³R⁴)(C═O)— | —NR¹¹— | —(C═O)(CH═CH)— | — | 1 |
| —O— | —(CH₂)ₙ— | —NR¹¹— | —(C═O)(CH═CH)— | — | 1 |
| —O— | 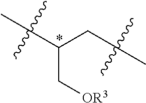 * R, S, or racemic | —NR¹¹— | —(C═O)(CH═CH)— | — | 1 |
| —O— | 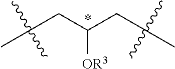 * R, S, or racemic | —NR¹¹— | —(C═O)(CH═CH)— | — | 1 |
| —O— | 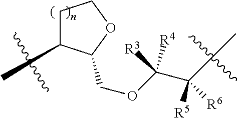 | —NR¹¹— | —(C═O)(CH═CH)— | — | 1 |
| —NR¹⁰— | —(CH₂)₃— | —NR¹¹— | —(C═O)(CH═CH)— | — | 1 |
| — | 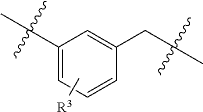 | —NR¹¹— | —(C═O)(CH═CH)— | — | 1 |
| — | 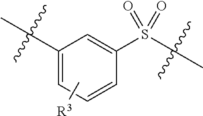 | —NR¹¹— | —(C═O)(CH═CH)— | — | 1 |
| — | 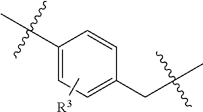 | —NR¹¹— | —(C═O)(CH═CH)— | — | 1 |
| — | 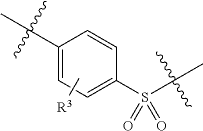 | —NR¹¹— | —(C═O)(CH═CH)— | — | 1 |
| — | 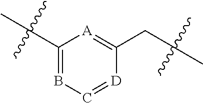 | —NR¹¹— | —(C═O)(CH═CH)— | — | 1 |
| — | 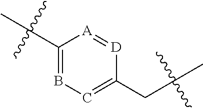 | —NR¹¹— | —(C═O)(CH═CH)— | — | 1 |
| — | 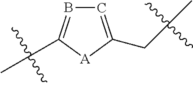 | —NR¹¹— | —(C═O)(CH═CH)— | — | 1 |
| — | 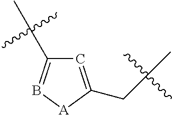 | —NR¹¹— | —(C═O)(CH═CH)— | — | 1 |
| — | 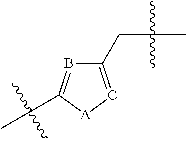 | —NR¹¹— | —(C═O)(CH═CH)— | — | 1 |
| —NR¹⁰— | 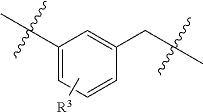 | —NR¹¹— | —(C═O)(CH═CH)— | — | 1 |
| —NR¹⁰— | 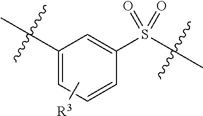 | —NR¹¹— | —(C═O)(CH═CH)— | — | 1 |
| —NR¹⁰— | 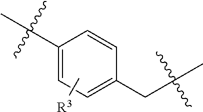 | —NR¹¹— | —(C═O)(CH═CH)— | — | 1 |
| —NR¹⁰— | 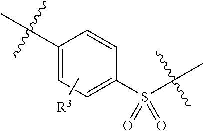 | —NR¹¹— | —(C═O)(CH═CH)— | — | 1 |
| —NR¹⁰— | 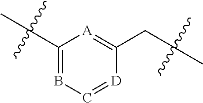 | —NR¹¹— | —(C═O)(CH═CH)— | — | 1 |
| —NR¹⁰— | 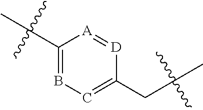 | —NR¹¹— | —(C═O)(CH═CH)— | — | 1 |
| —NR¹⁰— | 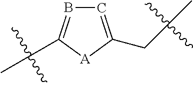 | —NR¹¹— | —(C═O)(CH═CH)— | — | 1 |
| —NR¹⁰— | 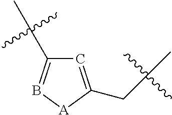 | —NR¹¹— | —(C═O)(CH═CH)— | — | 1 |
| —NR¹⁰— | 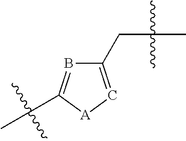 | —NR¹¹— | —(C═O)(CH═CH)— | — | 1 |
| — | C═O | —NH— | CH₂(CH═CH) | — | 1 |
| — | —(CH₂)(C═O)— | —NH— | CH₂(CH═CH) | — | 1 |
| — | —(CH₂)₂(C═O)— | —NH— | CH₂(CH═CH) | — | 1 |
| — | —(CH₂)₃(C═O)— | —NH— | CH₂(CH═CH) | — | 1 |
| — | —(CH₂)NH(C═O)— | —NH— | CH₂(CH═CH) | — | 1 |
| — | 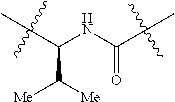 | —NH— | CH₂(CH═CH) | — | 1 |
| — | 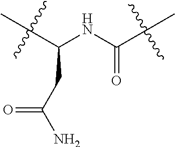 | —NH— | CH₂(CH═CH) | — | 1 |
| —O— | —(CH₂)₁(C═O)— | —NH— | CH₂(CH═CH) | — | 1 |
| —O— | —(CH₂)₂(C═O)— | —NH— | CH₂(CH═CH) | — | 1 |
| —O— | —(CH₂)₃(C═O)— | —NH— | CH₂(CH═CH) | — | 1 |
| —O— | 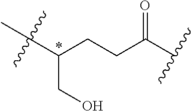 * R, S, or racemic | —NH— | CH₂(CH═CH) | — | 1 |
| —O— | 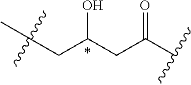 * R, S, or racemic | —NH— | CH₂(CH═CH) | | 1 |
| —O— | 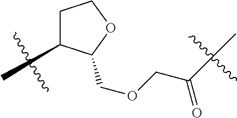 | —NH— | CH₂(CH═CH) | — | 1 |
| —O— | —(CH₂)₃— | —NH— | CH₂(CH═CH) | — | 1 |
| —NH— | —(CH₂)₂(C═O)— | —NH— | CH₂(CH═CH) | — | 1 |
| —NMe— | —(CH₂)₂(C═O)— | —NH— | CH₂(CH═CH) | — | 1 |
| — | 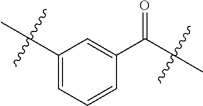 | —NH— | CH₂(CH═CH) | — | 1 |
| — | 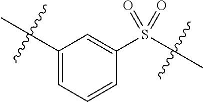 | —NH— | CH₂(CH═CH) | — | 1 |
| — | 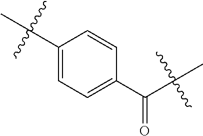 | —NH— | CH₂(CH═CH) | — | 1 |
| — | 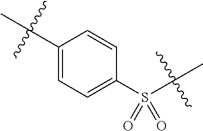 | —NH— | CH₂(CH═CH) | — | 1 |
| — | 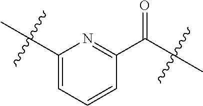 | —NH— | CH₂(CH═CH) | — | 1 |
| — | 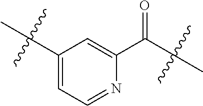 | —NH— | CH₂(CH═CH) | — | 1 |
| — | 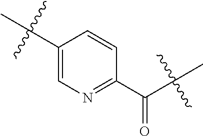 | —NH— | CH₂(CH═CH) | — | 1 |
| — | 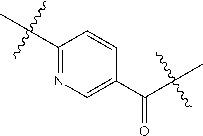 | —NH— | CH₂(CH═CH) | — | 1 |
| —NH— | 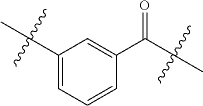 | —NH— | CH₂(CH═CH) | — | 1 |
| —NH— | 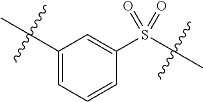 | —NH— | CH₂(CH═CH) | — | 1 |
| —NH— | 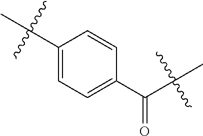 | —NH— | CH₂(CH═CH) | — | 1 |
| —NH— | 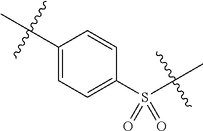 | —NH— | CH₂(CH═CH) | — | 1 |
| —NH— | 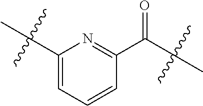 | —NH— | CH₂(CH═CH) | — | 1 |
| —NH— | 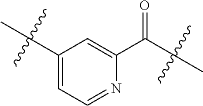 | —NH— | CH₂(CH═CH) | — | 1 |
| —NH— | 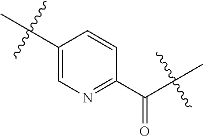 | —NH— | CH₂(CH═CH) | — | 1 |
| —NH— | 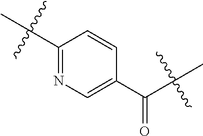 | —NH— | CH₂(CH═CH) | — | 1 |
| — | —(CH₂)— | —NH— | —(C═O)(CH═CH)— | — | 1 |
| — | 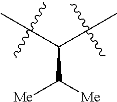 | —NH— | —(C═O)(CH═CH)— | — | 1 |
| — | 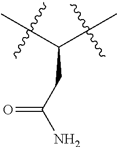 | —NH— | —(C═O)(CH═CH)— | — | 1 |
| — | —(CH₂)₂— | —NH— | —(C═O)(CH═CH)— | — | 1 |
| — | —(CH₂)₃— | —NH— | —(C═O)(CH═CH)— | — | 1 |
| — | —(CH₂)₄— | —NH— | —(C═O)(CH═CH)— | — | 1 |
| —O— | —(CH₂)₂— | —NH— | —(C═O)(CH═CH)— | — | 1 |
| —O— | —(CH₂)₃— | —NH— | —(C═O)(CH═CH)— | — | 1 |
| —O— | —(CH₂)₄— | —NH— | —(C═O)(CH═CH)— | — | 1 |
| —O— | 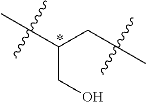 * R, S, or racemic | —NH— | —(C═O)(CH═CH)— | — | 1 |
| —O— | 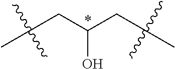 * R, S, or racemic | —NH— | —(C═O)(CH═CH)— | — | 1 |
| —O— | 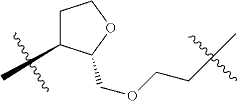 | —NH— | —(C═O)(CH═CH)— | — | 1 |
| —NH— | —(CH₂)₃— | —NH— | —(C═O)(CH═CH)— | — | 1 |
| —NH— | —(CH₂)₃— | —NH— | —(C═O)(CH═CH)— | — | 1 |
| —NMe— | —(CH₂)₃— | —NH— | —(C═O)(CH═CH)— | — | 1 |
| — | 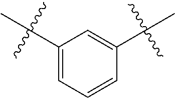 | —NH— | —(C═O)(CH═CH)— | — | 1 |
| — | 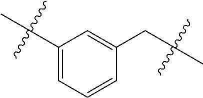 | —NH— | —(C═O)(CH═CH)— | — | 1 |
| — | 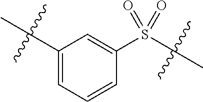 | —NH— | —(C═O)(CH═CH)— | — | 1 |
| — | 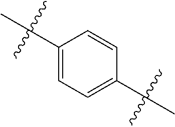 | —NH— | —(C═O)(CH═CH)— | — | 1 |
| — | 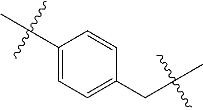 | —NH— | —(C═O)(CH═CH)— | — | 1 |
| — | 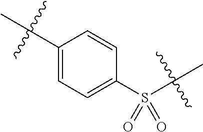 | —NH— | —(C═O)(CH═CH)— | — | 1 |
| — | 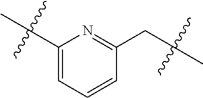 | —NH— | —(C═O)(CH═CH)— | — | 1 |
| — | 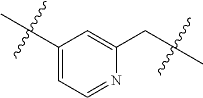 | —NH— | —(C═O)(CH═CH)— | — | 1 |
| — | 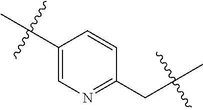 | —NH— | —(C═O)(CH═CH)— | — | 1 |
| — | 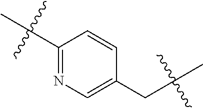 | —NH— | —(C═O)(CH═CH)— | — | 1 |
| —NH— | 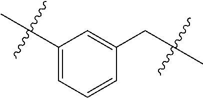 | —NH— | —(C═O)(CH═CH)— | — | 1 |
| —NH— | 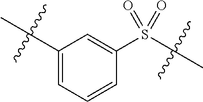 | —NH— | —(C═O)(CH═CH)— | — | 1 |
| —NH— | 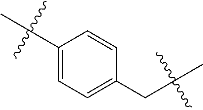 | —NH— | —(C═O)(CH═CH)— | — | 1 |
| —NH— | 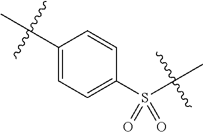 | —NH— | —(C═O)(CH═CH)— | — | 1 |
| —NH— | 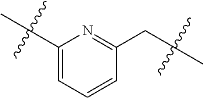 | —NH— | —(C═O)(CH═CH)— | — | 1 |
| —NH— | 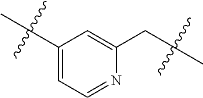 | —NH— | —(C═O)(CH═CH)— | — | 1 |
| —NH— | 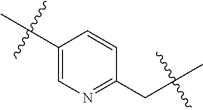 | —NH— | —(C═O)(CH═CH)— | — | 1 |
| —NH— | 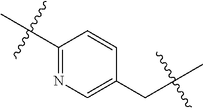 | —NH— | —(C═O)(CH═CH)— | — | 1 |
| — | —(CR³R⁴)(C═O)— | —NR¹¹— | —(CH₂)₃— | — | 1 |
| — | —(CH₂)ₙ(C═O)— | —NR¹¹— | —(CH₂)₃— | — | 1 |
| — | —(CH₂)O(CH₂)(C═O)— | —NR¹¹— | —(CH₂)₃— | — | 1 |
| — | —(CH₂)NR¹(CH)₂(C═O)— | —NR¹¹— | —(CH₂)₃— | — | 1 |
| — | —(CH₂)NR¹(C═O)— | —NR¹¹— | —(CH₂)₃— | — | 1 |
| — | 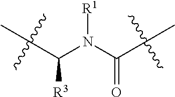 | —NR¹¹— | —(CH₂)₃— | — | 1 |
| —O— | —(CH₂)₁(C═O)— | —NR¹¹— | —(CH₂)₃— | — | 1 |
| —NR¹⁰— | —(CH₂)₂(C═O)— | —NR¹¹— | —(CH₂)₃— | — | 1 |
| Any | Any | Any | —(CH₂)₃— | — | 1 |
| — | 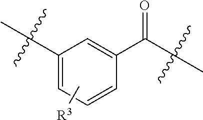 | —NR¹¹— | —(CH₂)₃— | — | 1 |
| — | 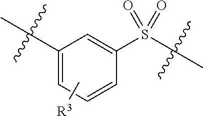 | —NR¹¹— | —(CH₂)₃— | — | 1 |
| — | 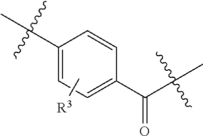 | —NR¹¹— | —(CH₂)₃— | — | 1 |
| — | 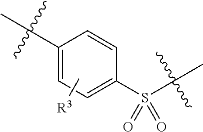 | —NR¹¹— | —(CH₂)₃— | — | 1 |
| — | 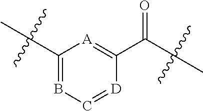 | —NR¹¹— | —(CH₂)₃— | — | 1 |
| — | 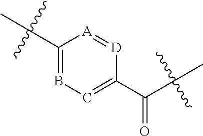 | —NR¹¹— | —(CH₂)₃— | — | 1 |
| — | 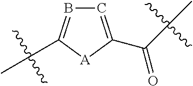 | —NR¹¹— | —(CH₂)₃— | — | 1 |
| — | 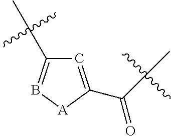 | —NR¹¹— | —(CH₂)₃— | — | 1 |
| — | 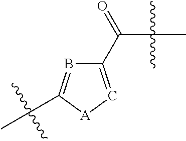 | —NR¹¹— | —(CH₂)₃— | — | 1 |
| —NR¹⁰— | 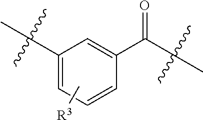 | —NR¹¹— | —(CH₂)₃— | — | 1 |
| —NR¹⁰— | 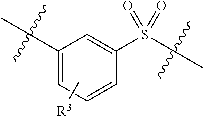 | —NR¹¹— | —(CH₂)₃— | — | 1 |
| —NR¹⁰— | 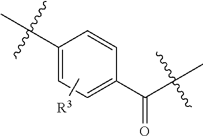 | —NR¹¹— | —(CH₂)₃— | — | 1 |
| —NR¹⁰— | 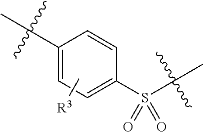 | —NR¹¹— | —(CH₂)₃— | — | 1 |
| —NR¹⁰— | 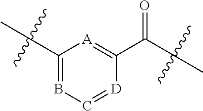 | —NR¹¹— | —(CH₂)₃— | — | 1 |
| —NR¹⁰— | 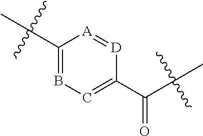 | —NR¹¹— | —(CH₂)₃— | — | 1 |
| —NR¹⁰— | 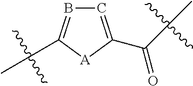 | —NR¹¹— | —(CH₂)₃— | — | 1 |
| —NR¹⁰— | 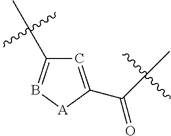 | —NR¹¹— | —(CH₂)₃— | — | 1 |
| —NR¹⁰— | 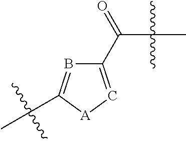 | —NR¹¹— | —(CH₂)₃— | — | 1 |
| —— | —(CH₂)ₙ— | —NR¹¹— | —(CH₂)₃— | — | 1 |
| —— | —(CR³R⁴)(C═O)— | —NR¹¹— | —(CH₂)₃— | — | 1 |
| —O— | n >= 2 | —NR¹¹— | —(CH₂)₃— | — | 1 |
| —O— | 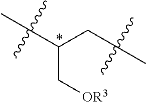 * R, S, or racemic | —NR¹¹— | —(CH₂)₃— | — | 1 |
| —O— | 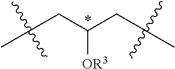 * R, S, or racemic | —NR¹¹— | —(CH₂)₃— | — | 1 |
| —O— | 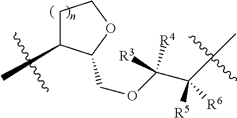 | —NR¹¹— | —(CH₂)₃— | — | 1 |
| —NR¹⁰— | —(CH₂)₃— | —NR¹¹— | —(CH₂)₃— | — | 1 |
| — | 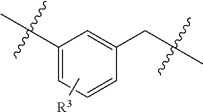 | —NR¹¹— | —(CH₂)₃— | — | 1 |
| — | 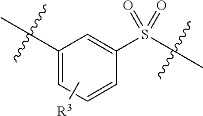 | —NR¹¹— | —(CH₂)₃— | — | 1 |
| — | 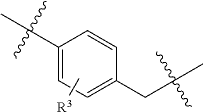 | —NR¹¹— | —(CH₂)₃— | — | 1 |
| — | 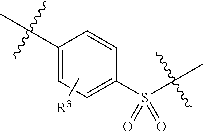 | —NR¹¹— | —(CH₂)₃— | — | 1 |
| — | 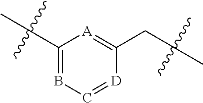 | —NR¹¹— | —(CH₂)₃— | — | 1 |
| — | 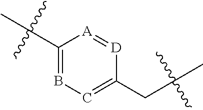 | —NR¹¹— | —(CH₂)₃— | — | 1 |
| — | 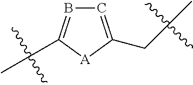 | —NR¹¹— | —(CH₂)₃— | — | 1 |
| — | 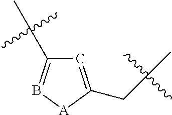 | —NR¹¹— | —(CH₂)₃— | — | 1 |
| — | 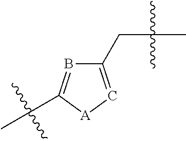 | —NR¹¹— | —(CH₂)₃— | — | 1 |
| —NR¹⁰— | 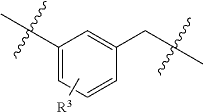 | —NR¹¹— | —(CH₂)₃— | — | 1 |
| —NR¹⁰— | 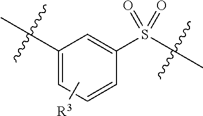 | —NR¹¹— | —(CH₂)₃— | — | 1 |
| —NR¹⁰— | 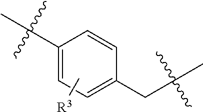 | —NR¹¹— | —(CH₂)₃— | — | 1 |
| —NR¹⁰— | 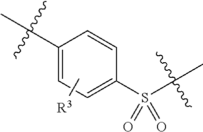 | —NR¹¹— | —(CH₂)₃— | — | 1 |
| —NR¹⁰— | 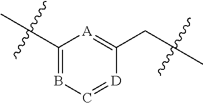 | —NR¹¹— | —(CH₂)₃— | — | 1 |
| —NR¹⁰— | 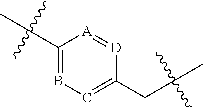 | —NR¹¹— | —(CH₂)₃— | — | 1 |
| —NR¹⁰— | 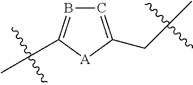 | —NR¹¹— | —(CH₂)₃— | — | 1 |
| —NR¹⁰— | 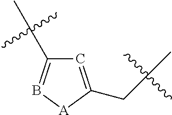 | —NR¹¹— | —(CH₂)₃— | — | 1 |
| —NR¹⁰— | 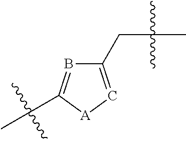 | —NR¹¹— | —(CH₂)₃— | — | 1 |

In some preferred embodiments, —X¹¹-L¹¹-X¹²-L¹²-X¹³—(CH₂)ₘ₁— is other than —O—CH₂—. In some embodiments, —X¹¹-L¹¹-X¹²-L¹²-X¹³—(CH₂)ₘ₁— is other than —(CH₂)_{b1}—(OCH₂CH₂)_{b2}—(CH₂)_{b3}—, wherein b1 and b3 are each integers between 0 and 4 and b2 is an integer between 0 and 2. For example, —X¹¹-L¹¹-X¹²-L¹²-X¹³—(CH₂)ₘ₁— is other than —(CH₂)₂—, or other than —(CH₂)₈—, or other than —(CH₂)₂—(OCH₂CH₂)—, or other than —(CH₂)₂—(OCH₂CH₂)₂—.

In some embodiments, the compounds of formula (A) have the structure of formula (IIIa)

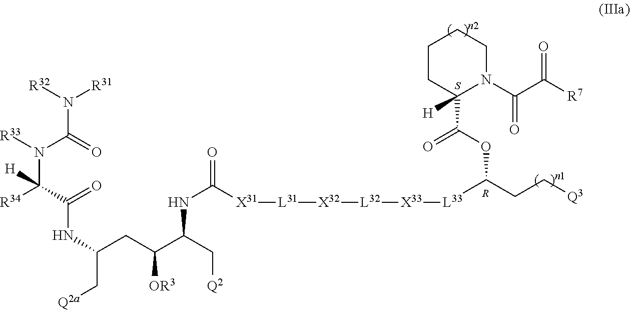

wherein, in formula (IIIa),

Q^{2a}, Q², Q³, n1, n2, R³, R⁷, R³¹, R³², R³³, and R³⁴ are as defined previously;

X³¹ is a linker selected from a bond and a hydrocarbylene group;

X³² and X³³ are independently selected from a linker selected from a bond, a hydrocarbylene group, and a functional linker group; and

L³¹, L³², and L³³ are independently selected from a bond, a hydrocarbylene group, and a functional linker group.

For example, R³¹ is selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, any of which may be substituted or unsubstituted. In some preferred embodiments, R³¹ is lower alkyl or substituted lower alkyl.

Also for example, R³² and R³³ are independently selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, any of which may be substituted or unsubstituted. In some preferred embodiments, R³² and R³³ are each lower alkyl or substituted lower alkyl. Also in some preferred embodiments, R³² and R³³ are taken together to form a ring. For example, R³² and R³³ may together form an ethylene, propylene, or butylene linker such that, together with the nitrogen atoms to which they attach, a 5-, 6-, or 7-member ring is formed. Such rings can have the structure

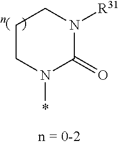

wherein the star represents the attachment point to the remainder of the compound.

Also for example, R³⁴ is selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, C₅-C₃₀ aryl, and C₅-C₃₀ heteroaryl, any of which may be substituted or unsubstituted. In some preferred embodiments, R³⁴ is lower alkyl or cycloalkyl, either of which may be substituted with, for example, a lower alkyl substituent. In other preferred embodiments, R³⁴ is substituted or unsubstituted aralkyl, such as an aryl-substituted benzyl group.

Also for example, L³³ is selected from a bond, alkylene such as methylene, and arylene. Examples of arylene groups are provided in Table 3.

For compounds having the structure of formula (IIIa), examples of the moiety —X³¹-L³¹-X³²-L³²-X³³-L³³- are provided in Table 3a.

TABLE 3a
Examples of —X³¹—L³¹—X³²—L³²—X³³—L³³— for compounds having the structure of formula (IIIa)

| X³¹ | L³¹ | X³² | L³² | X³³ | L³³ |
|---|---|---|---|---|---|
| — | — | — | (CH₂)ₙ | NH(C═O) | 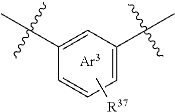 |
| — | — | NR^{X} | (CH₂)ₙ (n > 1) | NH(C═O) | 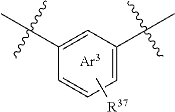 |
| — | — | — | (CH₂)ₙ | (C═O)NH | 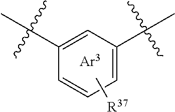 |
| — | — | — | —(CH₂)ₙ— | —NH— | 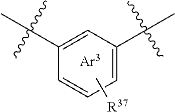 |
| — | — | NR^{X} | (CH₂)ₙ | (C═O)NH | 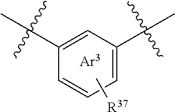 |
| — | — | NR^{X} | (CH₂)ₙ (n > 1) | —NH— | 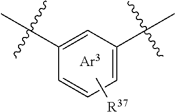 |
| — | — | — | (CH₂)ₙ | —O— | 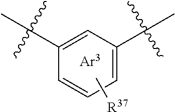 |
| — | — | NR^{X} | (CH₂)ₙ (n > 1) | —O— | 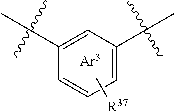 |
| — | — | — | (CH₂)ₙ | NH(C═O) | — |
| — | — | NR^{X} | (CH₂)ₙ (n > 1) | NH(C═O) | — |
| — | — | — | (CH₂)ₙ | (C═O)NH | — |
| — | — | — | (CH₂)ₙ | —NH— | — |
| — | — | NR^{X} | (CH₂)ₙ | (C═O)NH | — |
| — | — | NR^{X} | (CH₂)ₙ (n > 1) | —NH— | — |
| — | — | — | (CH₂)ₙ | —O— | — |
| — | — | NR^{X} | (CH₂)ₙ (n > 1) | —O— | — |
| — | —CR^{Y}R^{Z}— | NH(C═O) | (CH₂)ₙ | NH(C═O) | 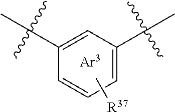 |
| — | —CR^{Y}R^{Z}— | NH(C═O)NR^{X} | (CH₂)ₙ (n > 1) | NH(C═O) | 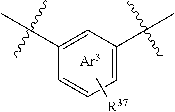 |
| — | —CR^{Y}R^{Z}— | NH(C═O) | (CH₂)ₙ | (C═O)NH | 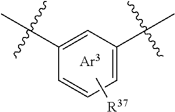 |
| — | —CR^{Y}R^{Z}— | NH(C═O) | (CH₂)ₙ | —NH— | 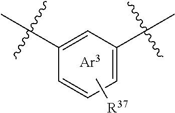 |
| — | —CR^{Y}R^{Z}— | NH(C═O)NR^{X} | (CH₂)ₙ | (C═O)NH | 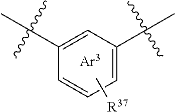 |
| — | —CR^{Y}R^{Z}— | NH(C═O)NR^{X} | (CH₂)ₙ (n > 1) | —NH— | 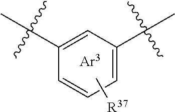 |
| — | —CR^{Y}R^{Z}— | NH(C═O) | (CH₂)ₙ | —O— | 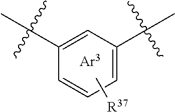 |
| — | —CR^{Y}R^{Z}— | NH(C═O)NR^{X} | (CH₂)ₙ (n > 1) | —O— | 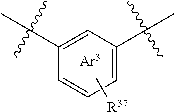 |
| — | —CR^{Y}R^{Z}— | NH(C═O) | (CH₂)ₙ | NH(C═O) | — |
| — | —CR^{Y}R^{Z}— | NH(C═O)NR^{X} | (CH₂)ₙ (n > 1) | NH(C═O) | — |
| — | —CR^{Y}R^{Z}— | NH(C═O) | (CH₂)ₙ | (C═O)NH | — |
| — | —CR^{Y}R^{Z}— | NH(C═O) | (CH₂)ₙ | —NH— | — |
| — | —CR^{Y}R^{Z}— | NH(C═O)NR^{X} | (CH₂)ₙ | (C═O)NH | — |
| — | —CR^{Y}R^{Z}— | NH(C═O)NR^{X} | (CH₂)ₙ (n > 1) | —NH— | — |
| — | —CR^{Y}R^{Z}— | NH(C═O) | (CH₂)ₙ | —O— | — |
| — | —CR^{Y}R^{Z}— | NH(C═O)NR^{X} | (CH₂)ₙ (n > 1) | —O— | — |
| 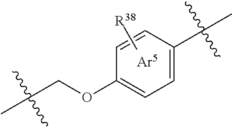 | — | — | (CH₂)ₙ | NH(C═O) | 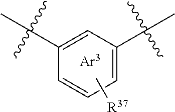 |
| 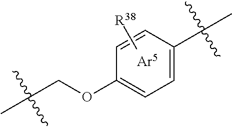 | (C═O) | NR^{X} | (CH₂)ₙ (n > 1) | NH(C═O) | 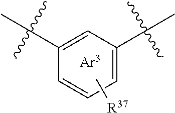 |
| 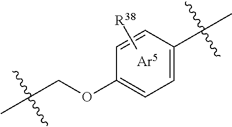 | — | — | (CH₂)ₙ | (C═O)NH | 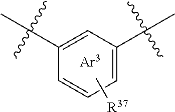 |
| 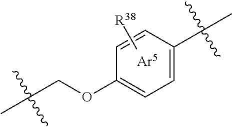 | — | — | (CH₂)ₙ | —NH— | 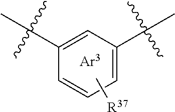 |
| 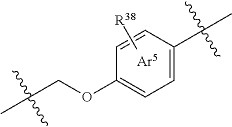 | (C═O) | NR^{X} | (CH₂)ₙ | (C═O)NH | 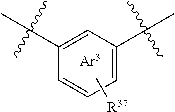 |
| 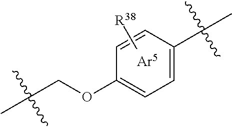 | (C═O) | NR^{X} | (CH₂)ₙ (n > 1) | —NH— | 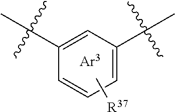 |
| 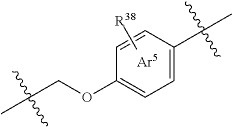 | — | — | (CH₂)ₙ | —O— | 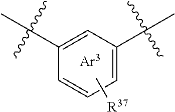 |
| 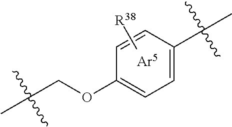 | (C═O) | NR^{X} | (CH₂)ₙ (n > 1) | —O— | 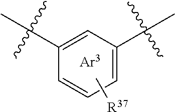 |
| 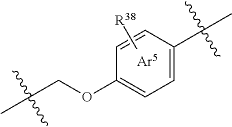 | — | — | (CH₂)ₙ | NH(C═O) | — |
| 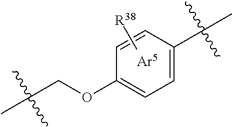 | (C═O) | NR^{X} | (CH₂)ₙ (n > 1) | NH(C═O) | — |
| 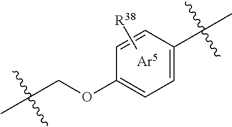 | — | — | (CH₂)ₙ | (C═O)NH | — |
| 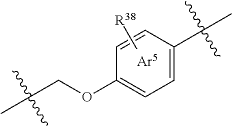 | — | — | (CH₂)ₙ | —NH— | — |
| 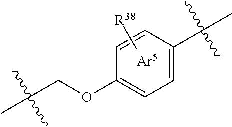 | (C═O) | NR^{X} | (CH₂)ₙ | (C═O)NH | — |
| 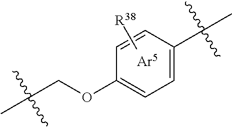 | (C═O) | NR^{X} | (CH₂)ₙ (n > 1) | —NH— | — |
| 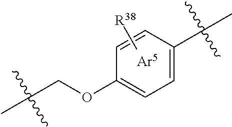 | — | — | (CH₂)ₙ | —O— | — |
| 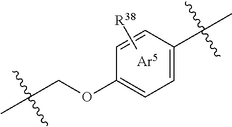 | (C═O) | NR^{X} | (CH₂)ₙ (n > 1) | —O— | — |

In Table 3a, R^{x}, R^{y}, and R^{z} are independently selected from H and hydrocarbyl. In some embodiments, R^{x}, R^{y}, and R^{z} are selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, C₅-C₃₀ aryl, and C₅-C₃₀ heteroaryl, any of which may be substituted or unsubstituted. In some preferred embodiments, R^{x}, R^{y}, and R^{z} are each lower alkyl.

In Table 3a, n is an integer greater than or equal to 1, unless otherwise specified.

In Table 3a, Ar³ is substituted or unsubstituted C₅-C₃₀ arylene, substituted or unsubstituted C₅-C₃₀ heteroarylene, substituted or unsubstituted C₅-C₃₀ biarylene, substituted or unsubstituted C₅-C₃₀ heterobiarylene, and substituted or unsubstituted C₁-C₃₀ alkylene. In some preferred embodiments, Ar³ is selected from biarylene and heterobiarylene moieties having a 6-member ring fused to a 5-membered ring, or a 6-member ring fused to another 6-member ring. Examples of Ar³ include phenyl, 2-pyridyl, 3-pyridyl, 4-pyridyl, 5-pyridyl, 6-pyridyl, 2-thiazolyl, 3-thiazolyl, 4-thiazolyl, and 5-thiazolyl. When Ar³ is substituted, it may contain 1, 2, 3, or 4 substituents (i.e., R³⁷ groups). Each R³⁷ is selected from alkyl and halo. For example, R³⁷ may be halo, such that Ar³ may contain 1, 2, or 3 halo substituents which may be the same or different. Preferred examples of Ar³ include aryl substituted as follows: 2-F, 4-F, 5-F, 6-F, 2-Cl, 4-Cl, 5-Cl, or 6-Cl, or a combination thereof.

In Table 3a, Ar⁵ is substituted or unsubstituted C₅-C₃₀ arylene, or substituted or unsubstituted C₅-C₃₀ heteroarylene. When Ar⁵ is substituted, it may contain 1, 2, 3, or 4 substituents (i.e., R³⁷ groups). Each R³⁸ is selected from alkyl and halo. For example, R³⁸ may be halo, such that Ar⁵ may contain 1, 2, or 3 halo substituents which may be the same or different. Preferred examples of Ar⁵ include aryl substituted as follows: 2,6-dimethyl, 2,6-difluoro, or 2,6-dichloro (wherein the numbers are with respect to the aryl position connected to oxygen).

In some embodiments, the compounds of formula (A) have the structure of formula (IVa)

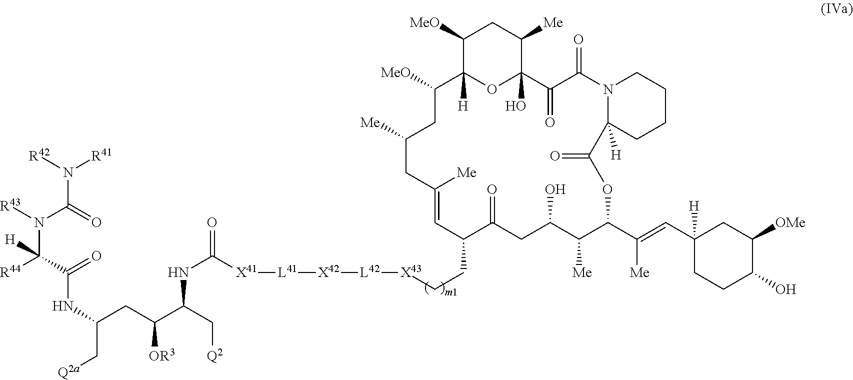

wherein, in formula (IVa):

m1 is an integer selected from 1 and 2

R³, Q^{2a} and Q² are as defined in formula (IIIa);

R⁴¹, R⁴², R⁴³, and R⁴⁴ are as defined for R³¹, R³², R³³, and R³⁴, respectively;

X⁴¹ is a linker selected from a bond and a hydrocarbylene group;

X⁴² and X⁴³ are independently selected from a linker selected from a bond, a hydrocarbylene group, and a functional linker group; and

L⁴¹, and L⁴² are independently selected from a bond, a hydrocarbylene group, and a functional linker group.

For compounds having the structure of formula (IVa), examples of the moiety —X⁴¹-L⁴¹-X⁴²-L⁴²-X⁴³—(CH₂)ₘ₁— are provided in Table 4a.

TABLE 4a
Examples of —X⁴¹—L⁴¹—X⁴²—L⁴²—X⁴³—(CH₂)ₘ₁—
for compounds having the structure of formula (IVa)

| X⁴¹ | L⁴¹ | X⁴² | L⁴² | X⁴³ | m1 |
|---|---|---|---|---|---|
| — | — | — | (CH₂)ₙ₂(CH═CH)ₘ (m = 0, n² = 0-2) (m = 1, n² = 1-2) | — | 1, 2 |
| — | — | — | (CH₂)ₙ₂ | —O— | 1, 2 |
| — | — | NR^{X} | (CH₂)ₙ₂(CH═CH)ₘ (m = 0, n² = 0-2) (m = 1, n² = 1-2) | — | 1, 2 |
| — | — | NR^{X} | (CH₂)ₙ₂ (n² not 1) | —O— | 1, 2 |
| — | (C═O)(CH₂) | NR^{X} | (CH₂)ₙ₂(CH═CH)ₘ (m = 0, n² = 0-2) (m = 1, n² = 1-2) | — | 1, 2 |
| — | (C═O)(CH₂) | NR^{X} | (CH₂)ₙ₂ (n² not 1) | —O— | 1, 2 |
| — | —NR^{Y}CR^{Y}R^{Z}(C═O)— | NR^{X} | (CH₂)ₙ₂(CH═CH)ₘ (m = 0, n² = 0-2) (m = 1, n² = 1-2) | — | 1, 2 |
| — | —NR^{Y}CR^{Y}R^{Z}(C═O)— | NR^{X} | (CH₂)ₙ₂ (n² not 1) | —O— | 1, 2 |
| — | —(C═O)CR^{Y}R^{Z}— | NR^{X} | (C═O)(CH₂)ₙ₂(CH═CH)ₘ (m = 0, 1, n² = 0-2) | — | 1, 2 |
| — | —(C═O)CR^{Y}R^{Z}— | NR^{X} | (C═O)(CH₂)ₙ₂ | —O— | 1, 2 |

| | | | | | |
|---|---|---|---|---|---|
| 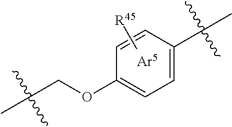 | — | — | (CH₂)ₙ₂(CH═CH)ₘ (m = 0, 1, n² = 0-2) | — | 1, 2 |
| 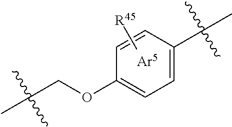 | (C═O) | — | (CH₂)ₙ₂ | —O— | 1, 2 |
| 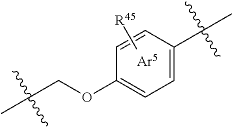 | (C═O) | NR^{X} | (CH₂)ₙ₂(CH═CH)ₘ (m = 0, n² = 0-2) (m = 1, n² = 1-2) | — | 1, 2 |
| 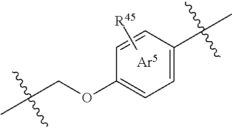 | (C═O) | NR^{X} | (CH₂)ₙ₂ (m = 0, n² = 0-2) (m = 1, n² = 1-2) | —O— | 1, 2 |
| 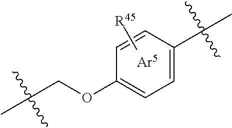 | (C═O)(CH₂) | NR^{X} | (CH₂)ₙ₂(CH═CH)ₘ (m = 0, n² = 0-2) (m = 1, n² = 1-2) | — | 1, 2 |
| 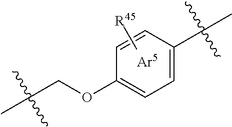 | (C═O)(CH₂) | NR^{X} | (CH₂)ₙ₂ (n² not 1) | —O— | 1, 2 |
| 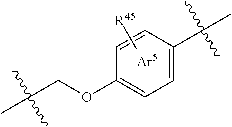 | —(C═O)NR^{Y}CR^{Y}R^{Z}(C═O)— | NR^{X} | (CH₂)ₙ₂(CH═CH)ₘ (m = 0, 1) | — | 1, 2 |
| 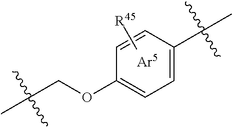 | —(C═O)NR^{Y}CR^{Y}R^{Z}(C═O)— | NR^{X} | (CH₂)ₙ₂ (n² not 1) | —O— | 1, 2 |
| 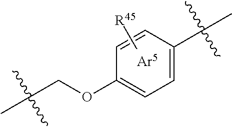 | —(C═O)CR^{Y}R^{Z}— | NR^{X} | (C═O)(CH₂)ₙ₂(CH═CH)ₘ (m = 0, 1) | — | 1, 2 |
| 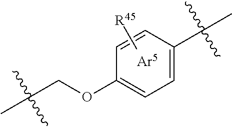 | —(C═O)CR^{Y}R^{Z}— | NR^{X} | (C═O)(CH₂)ₙ₂ | —O— | 1, 2 |

In Table 4a, R^{x}, R^{y}, R^{z}, and Ar⁵ are as defined previously, and R⁴⁵ is as defined for R³⁸ (see Table 3a). Also in Table 4a, and unless otherwise specified, m, and n2 are integers greater than or equal to 1.

In some preferred embodiments, —X⁴¹-L⁴¹-X⁴²-L⁴²-X⁴³—(CH₂)ₙ₁— is other than —O—(CH₂)—. In some embodiments, —X⁴¹-L⁴¹-X⁴²-L⁴²-X⁴³—(CH₂)ₙ₁— is other than —(CH₂)_{b1}—(OCH₂CH₂)_{b2}—(CH₂)_{b3}—, wherein b1 and b3 are each integers between 0 and 4 and b2 is an integer between 0 and 2. For example, —X⁴¹-L⁴¹-X⁴²-L⁴²-X⁴³—(CH₂)ₙ₁— is other than —(CH₂)₂—, or other than —(CH₂)₈—, or other than —(CH₂)₂—(OCH₂CH₂)—, or other than —(CH₂)₂—(OCH₂CH₂)₂—.

In some embodiments, the compounds of formula (A) have the structure of formula (Va)

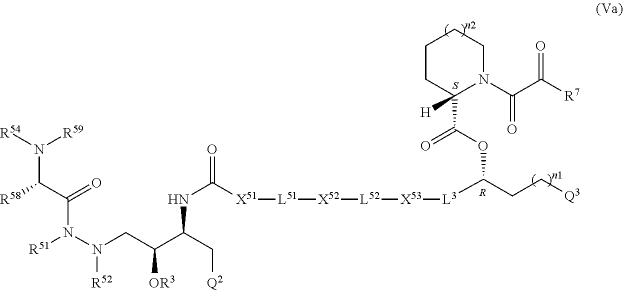

wherein, in formula (Va),

Q², Q³, n1, n2, R³, R⁷, R⁵¹, R⁵², R⁵⁴, R⁵⁸, R⁵⁹, and L³ are as defined previously;

X⁵¹ is selected from a bond, —O—, and —NR⁵⁶—;

L⁵¹ is alkylene;

X⁵² is selected from a bond, —O—, and —NR⁵⁶—;

L⁵² is alkylene;

X⁵³ is selected from —O— and —NR⁵⁶—; and

each R⁵⁶ is independently selected from H and alkyl.

For example, R⁵⁰ may be lower alkyl, including linear, branched, and cyclic alkyl, any of which may be substituted. For example, R⁵⁰ may be Me, Et, Pr, and the like.

Also for example, R⁵⁵ may be lower alkyl, such as Me, Et, Pr, and the like.

Also for example, Ar⁵ is aryl, including substituted aryl, heteroaryl, and substituted heteroaryl. As a further example, Ar⁵ may be pyridyl or pyrimidyl, and Ar⁵ may have the structure

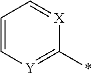

wherein

the star represents the attachment point to the remainder of the compound; and

X and Y are independently selected from —N— and —CH—.

Also for example, R⁵⁹ may be lower alkyl, including linear, branched, and cyclic alkyl, any of which may be substituted. For example, R⁵⁹ may be Me, Et, Pr, and the like.

Also for example, R⁵⁸ may have a structure such that R⁵⁸ and the adjacent carbonyl group form an L-amino acid residue. In some preferred embodiments, R⁵⁸ is selected from isopropyl, tert-butyl, 2-butyl, and —CH₂—S-Me. In other preferred embodiments, R⁵⁸ is selected from lower alkyl, including linear and branched, such as Me, Et, and the like. In still other embodiments, R⁵⁸ comprises a tertiary carbon with the formula —C(R^{m1})(R^{m2})(Ar⁶), wherein R^{m1} and R^{m2} are independently selected from H and alkyl, and wherein Ar⁶ is substituted or unsubstituted aryl, including heteroaryl. For example, R⁵⁸ may have the structure:

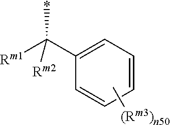

wherein n50 is an integer from 0 to 5 and each R^{m3} is independently selected from H, alkyl, and functional groups as listed herein previously. In some preferred embodiments, R^{m3} is selected from H, lower alkyl, halo, alkoxy, amido, and acetamido.

Also for example, R⁵¹ is selected from H and lower alkyl, including linear, branched, and cyclic alkyl, any of which may be substituted or unsubstituted.

Also for example, R⁵² is selected from H, alkyl, lower alkyl, aryl, heteroaryl, heteroatom-containing alkaryl, and heteroatom-containing aralkyl, any of which may be substituted or unsubstituted. For example, R⁵² may be linear, branched, or cyclic alkyl, including examples such as isopropyl and isobutyl. In some preferred embodiments, R⁵² is substituted aryl, including substituted heteroaryl, wherein the substituent(s) is/are selected from aryl, including heteroaryl. For example, R⁵² may have the structure —CH₂—Ar⁷-AR⁸, wherein Ar⁷ and Ar⁸ are independently selected from aryl and heteroaryl having one or more substituents. For example, R⁵² may have the structure

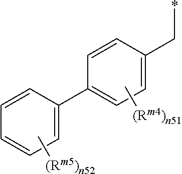

wherein

the star represents the attachment point to the remainder of the compound;

n51 is an integer in the range of 0-4;

n52 is an integer in the range of 0-5; and

each R^{m4} and R^{m5} is independently selected from alkyl, alkoxy, and functional groups. Some examples of R^{m5} include, for example, 4-Me, 4-CF3, 4-OH, 4-OMe, and 4-CN. In some preferred embodiments, R⁵² has a structure selected from

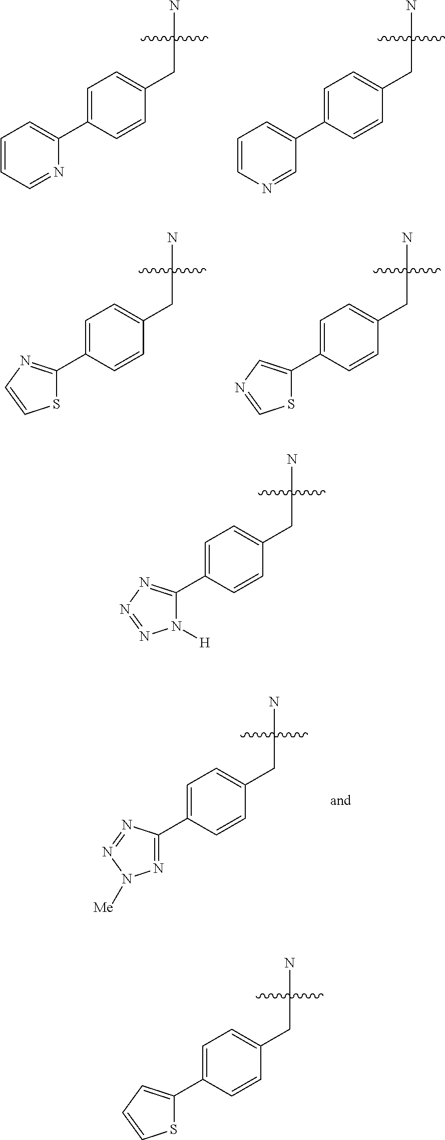

For compounds having the structure of formula (Va), examples of the moiety —X⁵¹-L⁵¹-X⁵²-L⁵²-X⁵³— are provided in Table 3.

TABLE 5
Examples of —X⁵¹—L⁵¹—X⁵²—L⁵²—X⁵³— for compounds having the structure of formula (Va)

| X⁵¹ | L⁵¹ | X⁵² | L⁵² | X⁵³ |
|---|---|---|---|---|
| — | —(CH₂)₂— | — | C═O | —NH— |
| — | —(CH₂)₃— | — | C═O | —NH— |
| — | C═O | —NH— | —(CH₂)₂— | —O— |
| — | —(CH₂)(C═O)— | —NH— | —(CH₂)₂— | —O— |
| — | —(CH₂)₂(C═O)— | —NH— | —(CH₂)₂— | —O— |
| — | —(CH₂)₃(C═O)— | —NH— | —(CH₂)₂— | —O— |
| — | —(CH₂)NH(C═O)— | —NH— | —(CH₂)₂— | —O— |
| — | 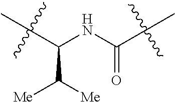 | —NH— | —(CH₂)₂— | —O— |
| — | 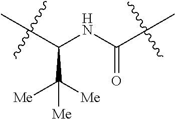 | —NH— | —(CH₂)₂— | —O— |
| — | 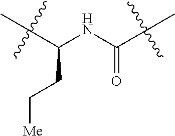 | —NH— | —(CH₂)₂— | —O— |
| — | 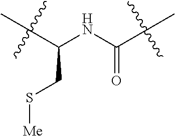 | —NH— | —(CH₂)₂— | —O— |
| — | 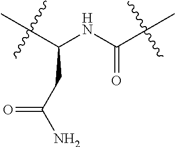 | —NH— | —(CH₂)₂— | —O— |
| —O— | —(CH₂)₁(C═O)— | —NH— | —(CH₂)₂— | —O— |
| —O— | —(CH₂)₂(C═O)— | —NH— | —(CH₂)₂— | —O— |
| —O— | —(CH₂)₃(C═O)— | —NH— | —(CH₂)₂— | —O— |
| —O— | —(CH₂)₃— | —NH— | —(CH₂)₂— | —O— |
| —NH— | —(CH₂)₂(C═O)— | —NH— | —(CH₂)₂— | —O— |
| —NMe— | —(CH₂)₂(C═O)— | —NH— | —(CH₂)₂— | —O— |
| — | —(CH₂)₃(C═O)— | —NH— | —(CH₂)₂— | —O— |
| — | —(CH₂)NH(C═O)— | —O— | —(CH₂)₂— | —O— |
| — | 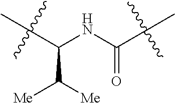 | —O— | —(CH₂)₂— | —O— |
| — | 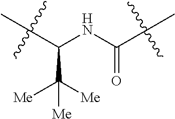 | —O— | —(CH₂)₂— | —O— |
| — | 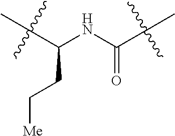 | —O— | —(CH₂)₂— | —O— |
| — | 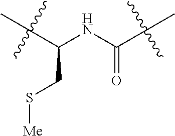 | —O— | —(CH₂)₂— | —O— |
| — | 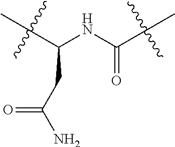 | —O— | —(CH₂)₂— | —O— |
| —O— | —(CH₂)₁(C═O)— | —O— | —(CH₂)₂— | —O— |
| —O— | —(CH₂)₂(C═O)— | —O— | —(CH₂)₂— | —O— |
| —O— | —(CH₂)₃(C═O)— | —O— | —(CH₂)₂— | —O— |
| —O— | —(CH₂)₃— | —O— | —(CH₂)₂— | —O— |
| —NH— | —(CH₂)₂(C═O)— | —O— | —(CH₂)₂— | —O— |
| —NMe— | —(CH₂)₂(C═O)— | —O— | —(CH₂)₂— | —O— |
| — | —(CH₂)NH(C═O)— | —O— | —(CH₂)₃— | —O— |
| — | 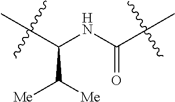 | —O— | —(CH₂)₃— | —O— |
| — | 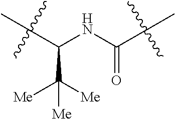 | —O— | —(CH₂)₃— | —O— |
| — | 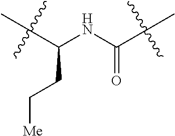 | —O— | —(CH₂)₃— | —O— |
| — | 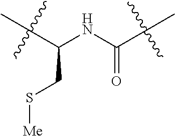 | —O— | —(CH₂)₃— | —O— |
| — | 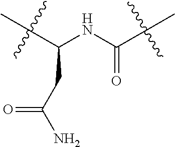 | —O— | —(CH₂)₃— | —O— |
| —O— | —(CH₂)₁(C═O)— | —O— | —(CH₂)₃— | —O— |
| —O— | —(CH₂)₂(C═O)— | —O— | —(CH₂)₃— | —O— |
| —O— | —(CH₂)₃(C═O)— | —O— | —(CH₂)₃— | —O— |
| —O— | —(CH₂)₃— | —O— | —(CH₂)₃— | —O— |
| —NH— | —(CH₂)₂(C═O)— | —O— | —(CH₂)₃— | —O— |
| —NMe— | —(CH₂)₂(C═O)— | —O— | —(CH₂)₃— | —O— |
| — | —(CH₂)— | —NH— | —(C═O)(CH₂)— | —O— |
| — | 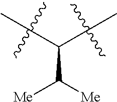 | —NH— | —(C═O)(CH₂)— | —O— |
| — | 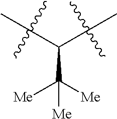 | —NH— | —(C═O)(CH₂)— | —O— |
| — | 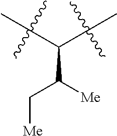 | —NH— | —(C═O)(CH₂)— | —O— |
| — | 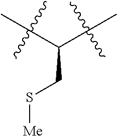 | —NH— | —(C═O)(CH₂)— | —O— |
| — | 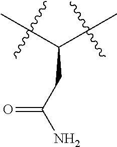 | —NH— | —(C═O)(CH₂)— | —O— |
| — | —(CH₂)₂— | —NH— | —(C═O)(CH₂)— | —O— |
| — | —(CH₂)₃— | —NH— | —(C═O)(CH₂)— | —O— |
| — | —(CH₂)₄— | —NH— | —(C═O)(CH₂)— | —O— |
| —O— | —(CH₂)₂— | —NH— | —(C═O)(CH₂)— | —O— |
| —O— | —(CH₂)₃— | —NH— | —(C═O)(CH₂)— | —O— |
| —O— | —(CH₂)₄— | —NH— | —(C═O)(CH₂)— | —O— |
| —NH— | —(CH₂)₃— | —NH— | —(C═O)(CH₂)— | —O— |
| —NH— | —(CH₂)₃— | —NH— | —(C═O)(CH₂)— | —O— |
| —NMe— | —(CH₂)₃— | —NH— | —(C═O)(CH₂)— | —O— |
| — | —(CH₂)ₙ— | — | C═O | —NR⁵⁶— |
| — | —(CH₂)O(CH₂)— | — | C═O | —NR⁵⁶— |
| — | (CH₂)NR¹(CH₂)— | — | C═O | —NR⁵⁶— |
| — | —(CR³R⁴)(C═O)— | —NR⁵⁶— | —(CH₂)₂— | —O— |
| — | —(CH₂)ₙ(C═O)— | —NR⁵⁶— | —(CH₂)₂— | —O— |
| — | —(CH₂)O(CH₂)(C═O)— | —NR⁵⁶— | C═O | —O— |
| — | —(CH₂)NR¹(CH)₂(C═O)— | —NR⁵⁶— | C═O | —O— |
| — | —(CH₂)NR¹(C═O)— | —NR⁵⁶— | —(CH₂)₂— | —O— |
| — | 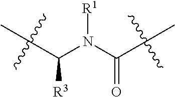 | —NR⁵⁶— | —(CH₂)₂— | —O— |
| —O— | —(CH₂)₁(C═O)— | —NR⁵⁶— | —(CH₂)₂— | —O— |
| —NR⁵⁶— | —(CH₂)₂(C═O)— | —NR⁵⁶— | —(CH₂)₂— | —O— |
| — | —(CR³R⁴)(C═O)— | —O— | —(CH₂)(CH₂)ₘ— | —O— |
| — | —(CH₂)ₙ(C═O)— | —O— | —(CH₂)(CH₂)ₘ— | —O— |
| — | —(CH₂)O(CH₂)(C═O)— | —O— | —(CH₂)(CH₂)ₘ— | —O— |
| — | —(CH₂)NR¹(C═O)— | —O— | —(CH₂)(CH₂)ₘ— | —O— |
| — | 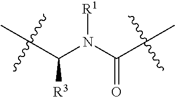 | —O— | —(CH₂)(CH₂)ₘ— | —O— |
| —O— | —(CH₂)₁(C═O)— | —O— | —(CH₂)(CH₂)ₘ— | —O— |
| —NR⁵⁶— | —(CH₂)₂(C═O)— | —O— | —(CH₂)(CH₂)ₘ— | —O— |
| — | —(CR³R⁴)(C═O)— | —O— | —(CH₂)(CH₂)ₘ— | —O— |
| — | —(CH₂)ₙ(C═O)— | —O— | —(CH₂)(CH₂)ₘ— | —O— |
| — | —(CH₂)O(CH₂)(C═O)— | —O— | —(CH₂)(CH₂)ₘ— | —O— |
| — | —(CH₂)NR¹(C═O)— | —O— | —(CH₂)(CH₂)ₘ— | —O— |
| — | 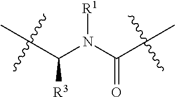 | —O— | —(CH₂)(CH₂)ₘ— | —O— |
| —O— | —(CH₂)n(C═O)— | —O— | —(CH₂)(CH₂)ₘ— | —O— |
| —NR⁵⁶— | —(CH₂)(CH₂)ₙ(C═O)— | —O— | —(CH₂)(CH₂)ₘ— | —O— |
| — | —(CH₂)ₙ— | —NR⁵⁶— | —(C═O)(CH₂)— | —O— |
| — | —(CR³R⁴)(C═O)— | —NR⁵⁶— | —(C═O)(CH₂)— | —O— |
| —O— | —(CH₂)ₙ— n >= 2 | —NR⁵⁶— | —(C═O)(CH₂)— | —O— |
| —O— | 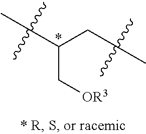 | —NR⁵⁶— | —(C═O)(CH₂)— | —O— |
| —O— | 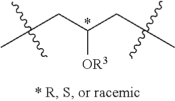 | —NR⁵⁶— | —(C═O)(CH₂)— | —O— |
| —NR⁵⁶— | —(CH₂)₃— | —NR⁵⁶— | —(C═O)(CH₂)— | —O— |

In some embodiments, the disclosure provides compounds having the structure of formula (VIa)

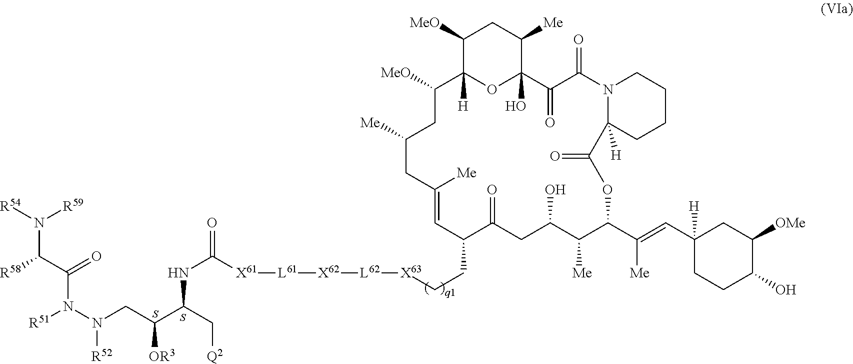

wherein, in formula (VIa):

Q², R³, R⁵¹, R⁵², R⁵⁴, R⁵⁸, and R⁵⁹, are as defined previously;

q1 is 1 or 2;

X⁶¹ is selected from a bond, —O—, and —NR⁶⁶—;

L⁶¹ is alkylene;

X⁶² is selected from a bond, —O—, and —NR⁶⁶—;

L⁶² is alkylene;

X⁶³ is selected from —O— and —NR⁶⁶—.

For compounds having the structure of formula (VIa), examples of the moiety —X⁶¹-L⁶¹-X⁶²-L⁶²-X⁶³—(CH₂)_{q1}— are provided in Table 4.

TABLE 6
Examples of —X⁶¹—L⁶¹—X⁶²—L⁶²—X⁶³—(CH₂)_{q1}—
for compounds having the structure of formula (VIa)

| X⁶¹ | L⁶¹ | X⁶² | L⁶² | X⁶³ | q1 |
|---|---|---|---|---|---|
| — | C═O | —NH— | —(CH₂)₂— | —O— | 1, 2 |
| — | —(CH₂)(C═O)— | —NH— | —(CH₂)₂— | —O— | 1, 2 |
| — | —(CH₂)₂(C═O)— | —NH— | —(CH₂)₂— | —O— | 1, 2 |
| — | —(CH₂)₃(C═O)— | —NH— | —(CH₂)₂— | —O— | 1, 2 |
| — | —(CH₂)NH(C═O)— | —NH— | —(CH₂)₂— | —O— | 1, 2 |
| — | 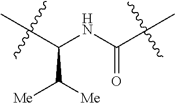 | —NH— | —(CH₂)₂— | —O— | 1, 2 |
| — | 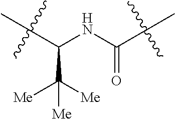 | —NH— | —(CH₂)₂— | —O— | 1, 2 |
| — | 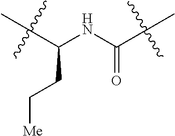 | —NH— | —(CH₂)₂— | —O— | 1, 2 |
| — | 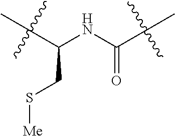 | —NH— | —(CH₂)₂— | —O— | 1, 2 |
| — | 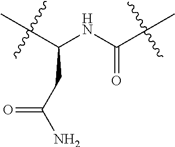 | —NH— | —(CH₂)₂— | —O— | 1, 2 |
| —O— | —(CH₂)₁(C═O)— | —NH— | —(CH₂)₂— | —O— | 1, 2 |
| —O— | —(CH₂)₂(C═O)— | —NH— | —(CH₂)₂— | —O— | 1, 2 |
| —O— | —(CH₂)₃(C═O)— | —NH— | —(CH₂)₂— | —O— | 1, 2 |
| —O— | 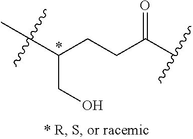 | —NH— | —(CH₂)₂— | —O— | 1, 2 |
| —O— | 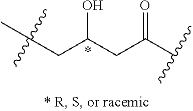 | —NH— | —(CH₂)₂— | —O— | 1, 2 |
| —O— | —(CH₂)₃— | —NH— | —(CH₂)₂— | —O— | 1, 2 |
| —NH— | —(CH₂)₂(C═O)— | —NH— | —(CH₂)₂— | —O— | 1, 2 |
| —NMe— | —(CH₂)₂(C═O)— | —NH— | —(CH₂)₂— | —O— | 1, 2 |
| — | —(CH₂)(C═O)— | —O— | —(CH₂)₂— | —O— | 1, 2 |
| — | —(CH₂)₂(C═O)— | —O— | —(CH₂)₂— | —O— | 1, 2 |
| — | —(CH₂)₃(C═O)— | —NH— | —(CH₂)₂— | —O— | 1, 2 |
| — | —(CH₂)NH(C═O)— | —O— | —(CH₂)₂— | —O— | 1, 2 |
| — | 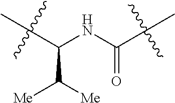 | —O— | —(CH₂)₂— | —O— | 1, 2 |
| — | 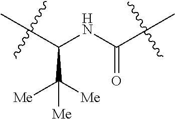 | —O— | —(CH₂)₂— | —O— | 1, 2 |
| — | 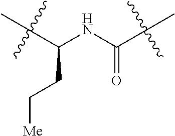 | —O— | —(CH₂)₂— | —O— | 1, 2 |
| — | 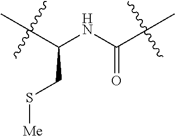 | —O— | —(CH₂)₂— | —O— | 1, 2 |
| — | 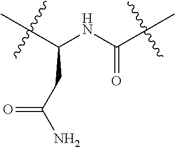 | —O— | —(CH₂)₂— | —O— | 1, 2 |
| —O— | —(CH₂)₁(C═O)— | —O— | —(CH₂)₂— | —O— | 1, 2 |
| —O— | —(CH₂)₂(C═O)— | —O— | —(CH₂)₂— | —O— | 1, 2 |
| —O— | —(CH₂)₃(C═O)— | —O— | —(CH₂)₂— | —O— | 1, 2 |
| —O— | 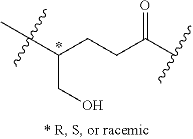 | —O— | —(CH₂)₂— | —O— | 1, 2 |
| —O— | 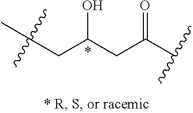 | —O— | —(CH₂)₂— | —O— | 1, 2 |
| —O— | —(CH₂)₃— | —O— | —(CH₂)₂— | —O— | 1, 2 |
| —NH— | —(CH₂)₂(C═O)— | —O— | —(CH₂)₂— | —O— | 1, 2 |
| —NMe— | —(CH₂)₂(C═O)— | —O— | —(CH₂)₂— | —O— | 1, 2 |
| — | —(CH₂)(C═O)— | —O— | —(CH₂)₃— | —O— | 1, 2 |
| — | —(CH₂)₂(C═O)— | —O— | —(CH₂)₃— | —O— | 1, 2 |
| — | —(CH₂)₃(C═O)— | —O— | —(CH₂)₃— | —O— | 1, 2 |
| — | —(CH₂)NH(C═O)— | —O— | —(CH₂)₃— | —O— | 1, 2 |
| — | 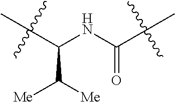 | —O— | —(CH₂)₃— | —O— | 1, 2 |
| — | 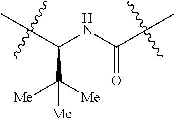 | —O— | —(CH₂)₃— | —O— | 1, 2 |
| — | 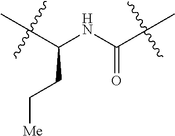 | —O— | —(CH₂)₃— | —O— | 1, 2 |
| — | 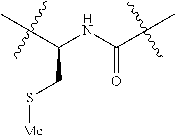 | —O— | —(CH₂)₃— | —O— | 1, 2 |
| — | 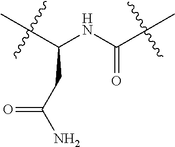 | —O— | —(CH₂)₃— | —O— | 1, 2 |
| —O— | —(CH₂)₁(C═O)— | —O— | —(CH₂)₃— | —O— | 1, 2 |
| —O— | —(CH₂)₂(C═O)— | —O— | —(CH₂)₃— | —O— | 1, 2 |
| —O— | —(CH₂)₃(C═O)— | —O— | —(CH₂)₃— | —O— | 1, 2 |
| —O— | 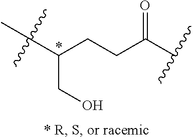 | —O— | —(CH₂)₃— | —O— | 1, 2 |
| —O— | 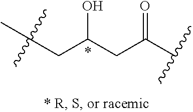 | —O— | —(CH₂)₃— | —O— | 1, 2 |
| —O— | —(CH₂)₃— | —O— | —(CH₂)₃— | —O— | 1, 2 |
| —NH— | —(CH₂)₂(C═O)— | —O— | —(CH₂)₃— | —O— | 1, 2 |
| —NMe— | —(CH₂)₂(C═O)— | —O— | —(CH₂)₃— | —O— | 1, 2 |
| — | —(CH₂)— | —NH— | —(C═O)(CH₂)— | —O— | 1, 2 |
| — | 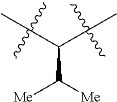 | —NH— | —(C═O)(CH₂)— | —O— | 1, 2 |
| — | 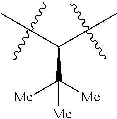 | —NH— | —(C═O)(CH₂)— | —O— | 1, 2 |
| — | 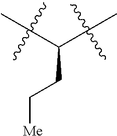 | —NH— | —(C═O)(CH₂)— | —O— | 1, 2 |
| — | 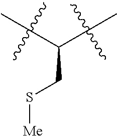 | —NH— | —(C═O)(CH₂)— | —O— | 1, 2 |
| — | 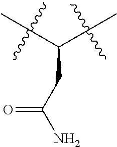 | —NH— | —(C═O)(CH₂)— | —O— | 1, 2 |
| — | —(CH₂)₂— | —NH— | —(C═O)(CH₂)— | —O— | 1, 2 |
| — | —(CH₂)₃— | —NH— | —(C═O)(CH₂)— | —O— | 1, 2 |
| — | —(CH₂)₄— | —NH— | —(C═O)(CH₂)— | —O— | 1, 2 |
| —O— | —(CH₂)₂— | —NH— | —(C═O)(CH₂)— | —O— | 1, 2 |
| —O— | —(CH₂)₃— | —NH— | —(C═O)(CH₂)— | —O— | 1, 2 |
| —O— | —(CH₂)₄— | —NH— | —(C═O)(CH₂)— | —O— | 1, 2 |
| —O— | 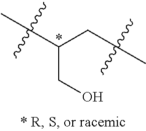 | —NH— | —(C═O)(CH₂)— | —O— | 1, 2 |
| —O— | 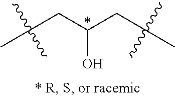 | —NH— | —(C═O)(CH₂)— | —O— | 1, 2 |
| —NH— | —(CH₂)₃— | —NH— | —(C═O)(CH₂)— | —O— | 1, 2 |
| —NH— | —(CH₂)₃— | —NH— | —(C═O)(CH₂)— | —O— | 1, 2 |
| —NMe— | —(CH₂)₃— | —NH— | —(C═O)(CH₂)— | —O— | 1, 2 |
| — | —(CR³R⁴)(C═O)— | —NR⁶⁶— | —(CH₂)₂— | —O— | 1, 2 |
| — | —(CH₂)ₙ(C═O)— | —NR⁶⁶— | —(CH₂)₂— | —O— | 1, 2 |
| — | —(CH₂)O(CH₂)(C═O)— | —NR⁶⁶— | C═O | —O— | 1, 2 |
| — | —(CH₂)NR¹(CH)₂(C═O)— | —NR⁶⁶— | C═O | —O— | 1, 2 |
| — | —(CH₂)NR¹(C═O)— | —NR⁶⁶— | —(CH₂)₂— | —O— | 1, 2 |
| — | 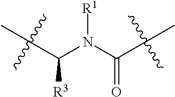 | —NR⁶⁶— | —(CH₂)₂— | —O— | 1, 2 |
| —O— | —(CH₂)₁(C═O)— | —NR⁶⁶— | —(CH₂)₂— | —O— | 1, 2 |
| —NR⁶⁶— | —(CH₂)₂(C═O)— | —NR⁶⁶— | —(CH₂)₂— | —O— | 1, 2 |
| — | —(CH₂)NR¹(C═O)— | —O— | —(CH₂)₂— | —O— | 1, 2 |
| — | 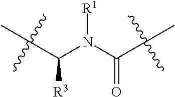 | —O— | —(CH₂)₂— | —O— | 1, 2 |
| —O— | —(CH₂)₁(C═O)— | —O— | —(CH₂)₂— | —O— | 1, 2 |
| —NR⁶⁶— | —(CH₂)₂(C═O)— | —O— | —(CH₂)₃— | —O— | 1, 2 |
| — | —(CH₂)NR¹(C═O)— | —O— | —(CH₂)₃— | —O— | 1, 2 |
| — | 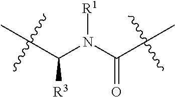 | —O— | —(CH₂)₃— | —O— | 1, 2 |
| —O— | —(CH₂)₁(C═O)— | —O— | —(CH₂)₃— | —O— | 1, 2 |
| —NR⁶⁶— | —(CH₂)₂(C═O)— | —O— | —(CH₂)₃— | —O— | 1, 2 |
| — | —(CH₂)ₙ— | —NR⁶⁶— | —(C═O)(CH₂)— | —O— | 1, 2 |
| — | —(CR³R⁴)(C═O)— | —NR⁶⁶— | —(C═O)(CH₂)— | —O— | 1, 2 |
| —O— | —(CH₂)ₙ— n >= 2 | —NR⁶⁶— | —(C═O)(CH₂)— | —O— | 1, 2 |
| —O— | 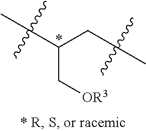 | —NR⁶⁶— | —(C═O)(CH₂)— | —O— | 1, 2 |
| —O— | 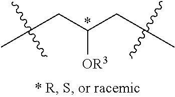 | —NR⁶⁶— | —(C═O)(CH₂)— | —O— | 1, 2 |
| —NR⁶⁶— | —(CH₂)₃— | —NR⁶⁶— | —(C═O)(CH₂)— | —O— | 1, 2 |
| — | C═O | —NH— | CH₂(CH═CH) | — | 1 |
| — | —(CH₂)(C═O)— | —NH— | CH₂(CH═CH) | — | 1 |
| — | —(CH₂)₂(C═O)— | —NH— | CH₂(CH═CH) | — | 1 |
| — | —(CH₂)₃(C═O)— | —NH— | CH₂(CH═CH) | — | 1 |
| — | —(CH₂)NH(C═O)— | —NH— | CH₂(CH═CH) | — | 1 |
| — | 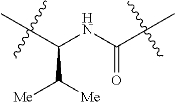 | —NH— | CH₂(CH═CH) | — | 1 |
| — | 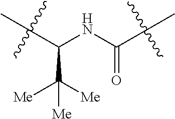 | —NH— | CH₂(CH═CH) | — | 1 |
| — | 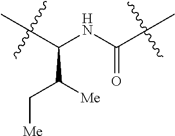 | —NH— | CH₂(CH═CH) | — | 1 |
| — | 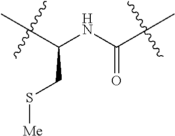 | —NH— | CH₂(CH═CH) | — | 1 |
| — | 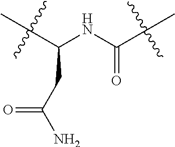 | —NH— | CH₂(CH═CH) | — | 1 |
| —O— | —(CH₂)₁(C═O)— | —NH— | CH₂(CH═CH) | — | 1 |
| —O— | —(CH₂)₂(C═O)— | —NH— | CH₂(CH═CH) | — | 1 |
| —O— | —(CH₂)₃(C═O)— | —NH— | CH₂(CH═CH) | — | 1 |
| —O— | 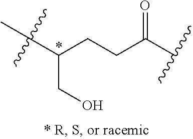 | —NH— | CH₂(CH═CH) | — | 1 |
| —O— | 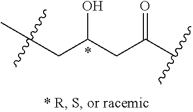 | —NH— | CH₂(CH═CH) | — | 1 |
| —O— | —(CH₂)₃— | —NH— | CH₂(CH═CH) | — | 1 |
| —NH— | —(CH₂)₂(C═O)— | —NH— | CH₂(CH═CH) | — | 1 |
| —NMe— | —(CH₂)₂(C═O)— | —NH— | CH₂(CH═CH) | — | 1 |
| — | —(CH₂)NH(C═O)— | —O— | CH₂(CH═CH) | — | 1 |
| — | 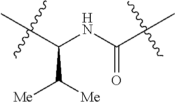 | —O— | CH₂(CH═CH) | — | 1 |
| — | 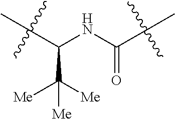 | —O— | CH₂(CH═CH) | — | 1 |
| — | 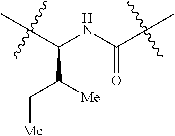 | —O— | CH₂(CH═CH) | — | 1 |
| — | 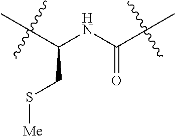 | —O— | CH₂(CH═CH) | — | 1 |
| — | 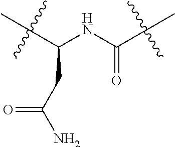 | —O— | CH₂(CH═CH) | — | 1 |
| — | —(CH₂)₂— | —NH— | —(C═O)(CH═CH)— | — | 1 |
| — | —(CH₂)₃— | —NH— | —(C═O)(CH═CH)— | — | 1 |
| — | —(CH₂)₄— | —NH— | —(C═O)(CH═CH)— | — | 1 |
| —O— | —(CH₂)₂— | —NH— | —(C═O)(CH═CH)— | — | 1 |
| —O— | —(CH₂)₃— | —NH— | —(C═O)(CH═CH)— | — | 1 |
| —O— | —(CH₂)₄— | —NH— | —(C═O)(CH═CH)— | — | 1 |
| —O— | 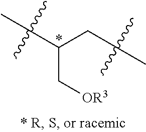 | —NH— | —(C═O)(CH═CH)— | — | 1 |
| —O— | 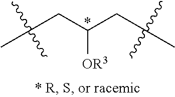 | —NH— | —(C═O)(CH═CH)— | — | 1 |
| —NH— | —(CH₂)₃— | —NH— | —(C═O)(CH═CH)— | — | 1 |
| —NH— | —(CH₂)₃— | —NH— | —(C═O)(CH═CH)— | — | 1 |
| —NMe— | —(CH₂)₃— | —NH— | —(C═O)(CH═CH)— | — | 1 |
| — | —(CR³R⁴)(C═O)— | —NR⁶⁶— | CH₂(CH═CH) | — | 1 |
| — | —(CH₂)ₙ(C═O)— | —NR⁶⁶— | CH₂(CH═CH) | — | 1 |
| — | —(CH₂)O(CH₂)(C═O)— | —NR⁶⁶— | CH₂(CH═CH) | — | 1 |
| — | —(CH₂)NR¹(CH)₂(C═O)— | —NR⁶⁶— | CH₂(CH═CH) | — | 1 |
| — | —(CH₂)NR¹(C═O)— | —NR⁶⁶— | CH₂(CH═CH) | — | 1 |
| — | 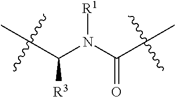 | —NR⁶⁶— | CH₂(CH═CH) | — | 1 |
| —O— | —(CH₂)₁(C═O)— | —NR⁶⁶— | CH₂(CH═CH) | — | 1 |
| —NR⁶⁶— | —(CH₂)₂(C═O)— | —NR⁶⁶— | CH₂(CH═CH) | — | 1 |
| — | —(CH₂)NR¹(C═O)— | —O— | CH₂(CH═CH) | — | 1 |
| — | 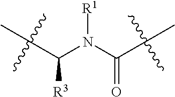 | —O— | CH₂(CH═CH) | — | 1 |
| — | —(CH₂)ₙ— | —NR⁶⁶— | —(C═O)(CH₂)— | — | 1 |
| — | —(CR³R⁴)(C═O)— | —NR⁶⁶— | —(C═O)(CH₂)— | — | 1 |
| —O— | —(CH₂)ₙ— n >= 2 | —NR⁶⁶— | —(C═O)(CH₂)— | — | 1 |
| — | —(CR³R⁴)(C═O)— | —O— | —(C═O)(CH₂)— | — | 1 |
| —O— | 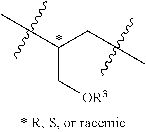 | —NR⁶⁶— | —(C═O)(CH₂)— | — | 1 |
| —O— | 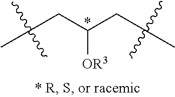 | —NR⁶⁶— | —(C═O)(CH₂)— | — | 1 |
| —NR⁶⁶— | —(CH₂)₃— | —NR⁶⁶— | —(C═O)(CH₂)— | — | 1 |
| —O— | —(CH₂)₃— | —NR⁶⁶— | —(C═O)(CH₂)— | — | 1 |
| —O— | —(CH₂)₃— | —NH— | CH₂(CH═CH) | — | 1 |
| —O— | —(CH₂)₃— | —O— | CH₂(CH═CH) | — | 1 |
| —NH— | —(CH₂)₂(C═O)— | —NH— | CH₂(CH═CH) | — | 1 |
| —NMe— | —(CH₂)₂(C═O)— | —NH— | CH₂(CH═CH) | — | 1 |
| — | —(CH₂)— | —NH— | —(C═O)(CH═CH)— | — | 1 |
| — | 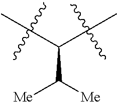 | —NH— | —(C═O)(CH═CH)— | — | 1 |
| — | 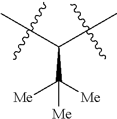 | —NH— | —(C═O)(CH═CH)— | — | 1 |
| — | 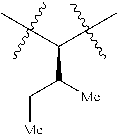 | —NH— | —(C═O)(CH═CH)— | — | 1 |
| — | 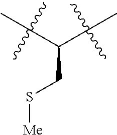 | —NH— | —(C═O)(CH═CH)— | — | 1 |
| — | 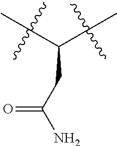 | —NH— | —(C═O)(CH═CH)— | — | 1 |
| — | —(CH₂)₂— | —NH— | —(C═O)(CH═CH)— | — | 1 |
| — | —(CH₂)₃— | —NH— | —(C═O)(CH═CH)— | — | 1 |
| — | —(CH₂)₄— | —NH— | —(C═O)(CH═CH)— | — | 1 |
| —O— | —(CH₂)₂— | —NH— | —(C═O)(CH═CH)— | — | 1 |
| —O— | —(CH₂)₃— | —NH— | —(C═O)(CH═CH)— | — | 1 |
| —O— | —(CH₂)₄— | —NH— | —(C═O)(CH═CH)— | — | 1 |
| —O— | 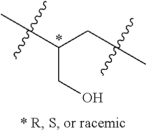 | —NH— | —(C═O)(CH═CH)— | — | 1 |
| —O— | 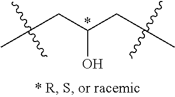 | —NH— | —(C═O)(CH═CH)— | — | 1 |
| —NH— | —(CH₂)₃— | —NH— | —(C═O)(CH═CH)— | — | 1 |
| —NH— | —(CH₂)₃— | —NH— | —(C═O)(CH═CH)— | — | 1 |
| —NMe— | —(CH₂)₃— | —NH— | —(C═O)(CH═CH)— | — | 1 |
| — | —(CR³R⁴)(C═O)— | —NR⁶⁶— | —(CH₂)₃— | — | 1 |
| — | —(CH₂)ₙ(C═O)— | —NR⁶⁶— | —(CH₂)₃— | — | 1 |
| — | —(CH₂)O(CH₂)(C═O)— | —NR⁶⁶— | —(CH₂)₃— | — | 1 |
| — | —(CH₂)NR¹(CH)₂(C═O)— | —NR⁶⁶— | —(CH₂)₃— | — | 1 |
| — | —(CH₂)NR¹(C═O)— | —NR⁶⁶— | —(CH₂)₃— | — | 1 |
| — | 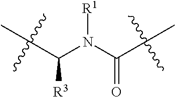 | —NR⁶⁶— | —(CH₂)₃— | — | 1 |
| —O— | —(CH₂)₁(C═O)— | —NR⁶⁶— | —(CH₂)₃— | — | 1 |
| —NR⁶⁶— | —(CH₂)₂(C═O)— | —NR⁶⁶— | —(CH₂)₃— | — | 1 |
| — | —(CH₂)ₙ— | —NR⁶⁶— | —(CH₂)₃— | — | 1 |
| — | —(CR³R⁴)(C═O)— | —NR⁶⁶— | —(CH₂)₃— | — | 1 |
| —O— | —(CH₂)ₙ— n >= 2 | —NR⁶⁶— | —(CH₂)₃— | — | 1 |
| —O— | 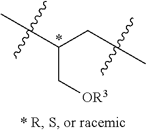 | —NR⁶⁶— | —(CH₂)₃— | — | 1 |
| —O— | 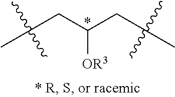 | —NR⁶⁶— | —(CH₂)₃— | — | 1 |
| —NR⁶⁶— | —(CH₂)₃— | —NR⁶⁶— | —(CH₂)₃— | — | 1 |
| — | —(CH₂)NR¹(C═O)— | —O— | —(CH₂)₃— | — | 1 |
| — | 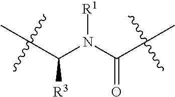 | —O— | —(CH₂)₃— | — | 1 |

In another embodiment, the disclosure provides compounds having the structure of formula (B)

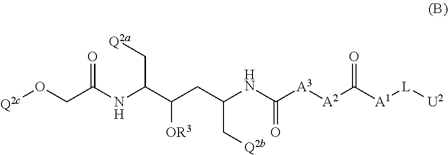

wherein

A¹ and A² are independently selected from nitrogen-containing linking moieties;

A³ is a hydrocarbylene linker;

Q^{2a}, Q^{2b}, and Q^{2c} are independently selected from alkyl and aryl;

R³ is selected from H, hydrocarbyl, functional groups, hydroxyl-protecting groups, and inorganic acid groups;

L is a linking moiety selected from hydrocarbylene and functional groups;

U² is a group selected from Units A, and B, as defined previously, as well as pharmaceutically acceptable salts, prodrugs, and metabolites thereof.

In some embodiments, the compounds of formula (B) have the structure of formula (IIIb):

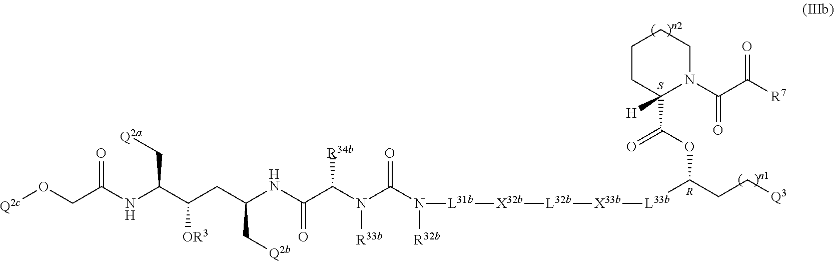

wherein, in formula (IIIb):

Q^{2a}, Q³, n1, n2, R³, and R⁷ are as defined previously (e.g., in formula (IIIa));

Q^{2b} and Q^{2c} are as defined for Q^{2a} previously;

R^{32b}, R^{33b}, and R^{34b} are independently selected from H and hydrocarbyl, provided that any two of R^{32b}, R^{33b}, and R^{34b} may be taken together to form a ring; and

X^{32b}, X^{33b}, L^{31b}, L^{32b}, and L^{33b} are linkers independently selected from a bond, a hydrocarbylene group, and a functional linker group.

For example, R^{32b} and R^{33b} are independently selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, any of which may be substituted or unsubstituted. In some preferred embodiments, R^{32b} and R^{33b} are each lower alkyl or substituted lower alkyl. Also in some preferred embodiments, R^{32b} and R^{33b} are taken together to form a ring. For example, R^{32b} and R^{33b} may together form an ethylene, propylene, or butylene linker such that, together with the nitrogen atoms to which they attach, a 5-, 6-, or 7-member ring is formed. Such rings can have the structure

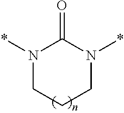

wherein the stars represent the attachment point to the remainder of the compound.

Also for example, R^{34b} is selected from H, C₁-C₂₄ alkyl, heteroatom-containing C₁-C₂₄ alkyl, C₂-C₂₄ alkenyl, heteroatom-containing C₂-C₂₄ alkenyl, C₂-C₂₄ alkynyl, heteroatom-containing C₂-C₂₄ alkynyl, C₅-C₃₀ aryl, and C₅-C₃₀ heteroaryl, any of which may be substituted or unsubstituted. In some preferred embodiments, R^{34b} is lower alkyl or cycloalkyl, either of which may be substituted with, for example, a lower alkyl substituent. In other preferred embodiments, R^{34b} is substituted or unsubstituted aralkyl, such as an aryl-substituted benzyl group.

Also for example, L^{33b} is selected from a bond, alkylene such as methylene, and arylene. Examples of arylene groups are provided in Table 3b.

For compounds having the structure of formula (IIIb), examples of the moiety —L^{31b}-X^{32b}-L^{32b}-X^{33b}-L^{33b}- are provided in Table 3b.

TABLE 3b
Examples of —L^{31b}—X^{32b}—L^{32b}—X^{33b}—L^{33b}— for compounds having the structure of formula (IIIb)

| L^{31b} | X^{32b} | L^{32b} | X^{33b} | L^{33b} |
|---|---|---|---|---|
| — | — | (CH₂)ₙ | — | 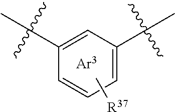 |
| — | — | (CH₂)ₙ (n not 1) | NH(C═O) | 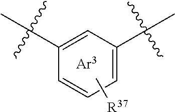 |
| — | — | (CH₂)ₙ (n not 1) | —NH— | 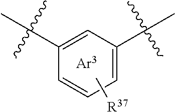 |
| — | — | (CH₂)ₙ | (C═O)NH | 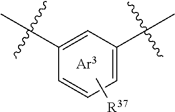 |
| — | — | (CH₂)ₙ (n not 1) | —O— | 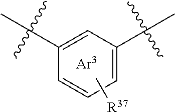 |
| — | — | (CH₂)ₙ | — | — |
| — | — | (CH₂)ₙ (n not 1) | NH(C═O) | — |
| — | — | (CH₂)ₙ (n not 1) | —NH— | — |
| — | — | (CH₂)ₙ | (C═O)NH | — |
| — | — | (CH₂)ₙ (n not 1) | —O— | — |
| —CR^{Y}R^{Z}— | (C═O)NR^{X} | (CH₂)ₙ | — | 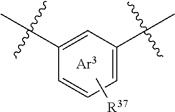 |
| —CR^{Y}R^{Z}— | (C═O)NR^{X} | (CH₂)ₙ (n not 1) | NH(C═O) | 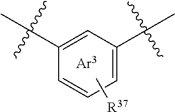 |
| —CR^{Y}R^{Z}— | (C═O)NR^{X} | (CH₂)ₙ (n not 1) | —NH— | 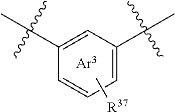 |
| —CR^{Y}R^{Z}— | (C═O)NR^{X} | (CH₂)ₙ | (C═O)NH | 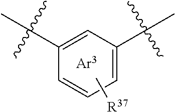 |
| —CR^{Y}R^{Z}— | (C═O)NR^{X} | (CH₂)ₙ (n not 1) | —O— | 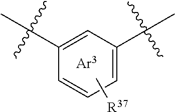 |
| —CR^{Y}R^{Z}— | (C═O)NR^{X} | (CH₂)ₙ | — | — |
| —CR^{Y}R^{Z}— | (C═O)NR^{X} | (CH₂)ₙ (n not 1) | NH(C═O) | — |
| —CR^{Y}R^{Z}— | (C═O)NR^{X} | (CH₂)ₙ (n not 1) | —NH— | — |
| —CR^{Y}R^{Z}— | (C═O)NR^{X} | (CH₂)ₙ | (C═O)NH | — |
| —CR^{Y}R^{Z}— | (C═O)NR^{X} | (CH₂)ₙ (n not 1) | —O— | — |

In Table 3b, R^{x}, R^{y}, R^{z}, n, Ar³, and R³⁷ are as defined previously (see Table 3a).

In some embodiments, the compounds of formula (B) have the structure of formula (IVb)

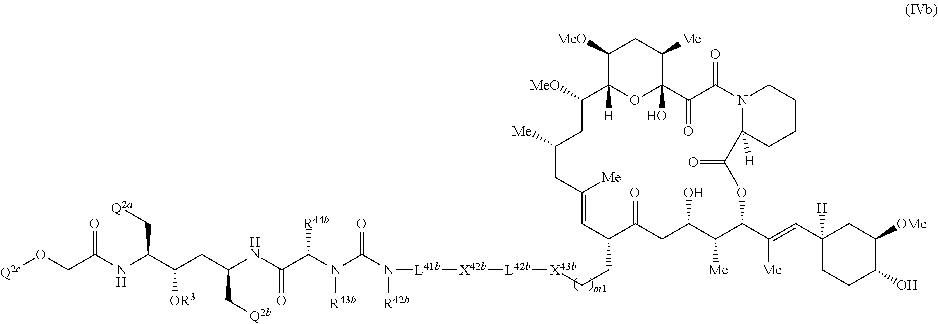

wherein, in formula (IVb):

m1 is an integer selected from 1 and 2;

R³, Q^{2a}, Q^{2b}, and Q^{2c} are as defined previously (e.g., in formula (IIIb) or in formula (IVa));

R^{42b}, R^{43b}, and R^{44b} are as defined for R^{32b}, R^{33b}, and R^{34b}, respectively, in formula (IIIb);

X^{42b} and X^{43b} are independently selected from a linker selected from a bond, a hydrocarbylene group, and a functional linker group; and

L^{41b}, and L^{42b} are independently selected from a bond, a hydrocarbylene group, and a functional linker group.

For compounds having the structure of formula (IVb), examples of the moiety -L^{41b}-X^{42b}-L^{42b}-X^{43b}—(CH₂)ₘ₁— are provided in Table 4b.

TABLE 4b
Examples of —L^{41b}—X^{42b}—L^{42b}—X^{43b}—(CH₂)ₘ₁—
for compounds having the structure of formula (IVb)

| L^{41b} | X^{42b} | L^{42b} | X^{43b} | m1 |
|---|---|---|---|---|
| — | — | (CH₂)ₙ₂(CH═CH)ₘ | — | 1, 2 |
| | | (m = 0, n² = 0-2) | | |
| | | (m = 1, n² = 1-2) | | |
| — | — | (CH₂)ₙ₂ | —O— | 1, 2 |
| — | — | (CH₂)ₙ₂(CH═CH)ₘ | — | 1, 2 |
| | | (m = 0, n² = 0-2) | | |
| | | (m = 1, n² = 1-2) | | |
| — | — | (CH₂)ₙ₂ | —O— | 1, 2 |
| | | (n² not 1) | | |
| —NR^{Y}CR^{Y}R^{Z}(C═O)— | NR^{X} | (CH₂)ₙ₂(CH═CH)ₘ | — | 1, 2 |
| | | (m = 0, n² = 0-2) | | |
| | | (m = 1, n² = 1-2) | | |
| —NR^{Y}CR^{Y}R^{Z}(C═O)— | NR^{X} | (CH₂)ₙ₂ | —O— | 1, 2 |
| | | (n² not 1) | | |
| —(C═O)CR^{Y}R^{Z}— | NR^{X} | (C═O)(CH₂)ₙ₂(CH═CH)ₘ | — | 1, 2 |
| | | (m = 0, 1, n² = 0-2) | | |
| —(C═O)CR^{Y}R^{Z}— | NR^{X} | (C═O)(CH₂)ₙ₂ | —O— | 1, 2 |

In Table 4b, R^{x}, R^{y}, R^{z}, m, and n2 are as defined previously (see Table 4a).

The compounds described herein may further be modified as described in PCT application PCT/US06/43400, which is entitled “Improving the Pharmacokinetics of Protease Inhibitors and Other Drugs,” and which published as WO 2007/053792, the contents of which are incorporated herein by reference.

A compound of the disclosure may be administered in the form of a salt, ester, amide, prodrug, active metabolite, analog, or the like, provided that the salt, ester, amide, prodrug, active metabolite or analog is pharmaceutically acceptable and pharmacologically active in the present context. Salts, esters, amides, prodrugs, active metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 5th Ed. (New York: Wiley-Interscience, 2001). Furthermore, where appropriate, functional groups on the compounds of the disclosure may be protected from undesired reactions during preparation or administration using protecting group chemistry. Suitable protecting groups are described, for example, in Green, Protective Groups in Organic Synthesis, 3rd Ed. (New York: Wiley-Interscience, 1999).

For example, where appropriate, any of the compounds described herein may be in the form of a pharmaceutically acceptable salt. A pharmaceutically acceptable salt may be prepared from any pharmaceutically acceptable organic acid or base, any pharmaceutically acceptable inorganic acid or base, or combinations thereof. The acid or base used to prepare the salt may be naturally occurring.

Suitable organic acids for preparing acid addition salts include, e.g., C₁-C₆ alkyl and C₆-C₁₂ aryl carboxylic acids, di-carboxylic acids, and tri-carboxylic acids such as acetic acid, propionic acid, succinic acid, maleic acid, fumaric acid, tartaric acid, glycolic acid, citric acid, pyruvic acid, oxalic acid, malic acid, malonic acid, benzoic acid, cinnamic acid, mandelic acid, salicylic acid, phthalic acid, and terephthalic acid, and aryl and alkyl sulfonic acids such as methanesulfonic acid, ethanesulfonic acid, and p-toluenesulfonic acid, and the like. Suitable inorganic acids for preparing acid addition salts include, e.g., hydrochloric acid, hydrobromic acid, hydroiodic acid, sulfuric acid, nitric acid, and phosphoric acid, and the like. An acid addition salt may be reconverted to the free base by treatment with a suitable base.

Suitable organic bases for preparing basic addition salts include, e.g., primary, secondary and tertiary amines, such as trimethylamine, triethylamine, tripropylamine, N,N-dibenzylethylenediamine, 2-dimethylaminoethanol, ethanolamine, ethylenediamine, glucamine, glucosamine, histidine, and polyamine resins, cyclic amines such as caffeine, N-ethylmorpholine, N-ethylpiperidine, and purine, and salts of amines such as betaine, choline, and procaine, and the like. Suitable inorganic bases for preparing basic addition salts include, e.g., salts derived from sodium, potassium, ammonium, calcium, ferric, ferrous, aluminum, lithium, magnesium, or zinc such as sodium hydroxide, potassium hydroxide, calcium carbonate, sodium carbonate, and potassium carbonate, and the like. A basic addition salt may be reconverted to the free acid by treatment with a suitable acid.

Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Any of the compounds of the disclosure may be the active agent in a formulation as described herein. Formulations containing the compounds of the disclosure may include 1, 2, 3 or more of the compounds described herein, and may also include one or more additional active agents such as analgesics, antibiotics, and other anti-retroviral agents (such as reverse transcriptase inhibitors including 3′-azido-2′,3′-dideoxythymidine (AZT), 2′3′-dideoxycytidine (ddC), and 2′3′-dideoxyinosine (ddI)).

The amount of active agent in the formulation typically ranges from about 0.05 wt % to about 95 wt % based on the total weight of the formulation. For example, the amount of active agent may range from about 0.05 wt % to about 50 wt %, or from about 0.1 wt % to about 25 wt %. Alternatively, the amount of active agent in the formulation may be measured so as to achieve a desired dose.

Formulations containing the compounds of the disclosure may be presented in unit dose form or in multi-dose containers with an optional preservative to increase shelf life.

The compositions of the disclosure may be administered to the patient by any appropriate method. In general, both systemic and localized methods of administration are acceptable. It will be obvious to those skilled in the art that the selection of a method of administration will be influenced by a number of factors, such as the condition being treated, frequency of administration, dosage level, and the wants and needs of the patient. For example, certain methods may be better suited for rapid delivery of high doses of active agent, while other methods may be better suited for slow, steady delivery of active agent. Examples of methods of administration that are suitable for delivery of the compounds of the disclosure include parental and transmembrane absorption (including delivery via the digestive and respiratory tracts). Formulations suitable for delivery via these methods are well known in the art.

For example, formulations containing the compounds of the disclosure may be administered parenterally, such as via intravenous, subcutaneous, intraperitoneal, or intramuscular injection, using bolus injection and/or continuous infusion. Generally, parenteral administration employs liquid formulations.

The compositions may also be administered via the digestive tract, including orally and rectally. Examples of formulations that are appropriate for administration via the digestive tract include tablets, capsules, pastilles, chewing gum, aqueous solutions, and suppositories.

The formulations may also be administered via transmucosal administration. Transmucosal delivery includes delivery via the oral (including buccal and sublingual), nasal, vaginal, and rectal mucosal membranes. Formulations suitable for transmucosal deliver are well known in the art and include tablets, chewing gums, mouthwashes, lozenges, suppositories, gels, creams, liquids, and pastes.

The formulations may also be administered transdermally. Transdermal delivery may be accomplished using, for example, topically applied creams, liquids, pastes, gels and the like as well as what is often referred to as transdermal “patches.”

The formulations may also be administered via the respiratory tract. Pulmonary delivery may be accomplished via oral or nasal inhalation, using aerosols, dry powders, liquid formulations, or the like. Aerosol inhalers and imitation cigarettes are examples of pulmonary dosage forms.

Liquid formulations include solutions, suspensions, and emulsions. For example, solutions may be aqueous solutions of the active agent and may include one or more of propylene glycol, polyethylene glycol, and the like. Aqueous suspensions can be made by dispersing the finely divided active agent in water with viscous material, such as natural or synthetic gums, resins, methylcellulose, sodium carboxymethylcellulose, or other well known suspending agents. Also included are formulations of solid form which are intended to be converted, shortly before use, to liquid form.

Tablets and lozenges may comprise, for example, a flavored base such as compressed lactose, sucrose and acacia or tragacanth and an effective amount of an active agent. Pastilles generally comprise the active agent in an inert base such as gelatin and glycerine or sucrose and acacia. Mouthwashes generally comprise the active agent in a suitable liquid carrier.

For topical administration to the epidermis the chemical compound according to the disclosure may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Transdermal patches typically comprise: (1) a impermeable backing layer which may be made up of any of a wide variety of plastics or resins, e.g. aluminized polyester or polyester alone or other impermeable films; and (2) a reservoir layer comprising, for example, a compound of the disclosure in combination with mineral oil, polyisobutylene, and alcohols gelled with USP hydroxymethylcellulose. As another example, the reservoir layer may comprise acrylic-based polymer adhesives with resinous crosslinking agents which provide for diffusion of the active agent from the reservoir layer to the surface of the skin. The transdermal patch may also have a delivery rate-controlling membrane such as a microporous polypropylene disposed between the reservoir and the skin. Ethylene-vinyl acetate copolymers and other microporous membranes may also be used. Typically, an adhesive layer is provided which may comprise an adhesive formulation such as mineral oil and polyisobutylene combined with the active agent.

Other typical transdermal patches may comprise three layers: (1) an outer layer comprising a laminated polyester film; (2) a middle layer containing a rate-controlling adhesive, a structural non-woven material and the active agent; and (3) a disposable liner that must be removed prior to use. Transdermal delivery systems may also involve incorporation of highly lipid soluble carrier compounds such as dimethyl sulfoxide (DMSO), to facilitate penetration of the skin. Other carrier compounds include lanolin and glycerin.

Rectal or vaginal suppositories comprise, for example, an active agent in combination with glycerin, glycerol monopalmitate, glycerol, monostearate, hydrogenated palm kernel oil and fatty acids. Another example of a suppository formulation includes ascorbyl palmitate, silicon dioxide, white wax, and cocoa butter in combination with an effective amount of an active agent.

Nasal spray formulations may comprise a solution of active agent in physiologic saline or other pharmaceutically suitable carder liquids. Nasal spray compression pumps are also well known in the art and can be calibrated to deliver a predetermined dose of the solution.

Aerosol formulations suitable for pulmonary administration include, for example, formulations wherein the active agent is provided in a pressurized pack with a suitable propellant. Suitable propellants include chlorofluorocarbons (CFCs) such as dichlorodifluoromethane, trichlorofluoromethane, or dichlorotetrafluoroethane, carbon dioxide, or other suitable gases. The aerosol may also contain a surfactant such as lecithin. The dose of drug may be controlled by provision of a metered valve.

Dry powder suitable for pulmonary administration include, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. Unit doses for dry powder formulations may be, for example, in the form of capsules or cartridges of, e.g., gelatin, or blister packs from which the powder may be administered by means of an inhaler.

In addition to the foregoing components, it may be necessary or desirable in some cases (depending, for instance, on the particular composition or method of administration) to incorporate any of a variety of additives, e.g., components that improve drug delivery, shelf-life, patient acceptance, etc. Suitable additives include acids, antioxidants, antimicrobials, buffers, colorants, crystal growth inhibitors, defoaming agents, diluents, emollients, fillers, flavorings, gelling agents, fragrances, lubricants, propellants, thickeners, salts, solvents, surfactants, other chemical stabilizers, or mixtures thereof. Examples of these additives can be found, for example, in M. Ash and I. Ash, Handbook of Pharmaceutical Additives (Hampshire, England: Gower Publishing, 1995), the contents of which are herein incorporated by reference.

Appropriate dose and regimen schedules will be apparent based on the present disclosure and on information generally available to the skilled artisan. When the compounds of the disclosure are used in the treatment of HIV, achievement of the desired effects may require weeks, months, or years of controlled, low-level administration of the formulations described herein. Other dosage regimens, including less frequent administration of high-intensity dosages, are also within the scope of the disclosure.

The amount of active agent in formulations that contain the compounds of the disclosure may be calculated to achieve a specific dose (i.e., unit weight of active agent per unit weight of patient) of active agent. Furthermore, the treatment regimen may be designed to sustain a predetermined systemic level of active agent. For example, formulations and treatment regimen may be designed to provide an amount of active agent that ranges from about 0.001 mg/kg/day to about 100 mg/kg/day for an adult. As a further example, the amount of active agent may range from about 0.1 mg/kg/day to about 50 mg/kg/day, about 0.1 mg/kg/day to about 25 mg/kg/day, or about 1 mg/kg/day to about 10 mg/kg/day. One of skill in the art will appreciate that dosages may vary depending on a variety of factors, including method and frequency of administration, and physical characteristics of the patient.

Treatment regimens that make use of multiple methods of administration are within the scope of the disclosure. For example, a small, steady dose of the compounds of the disclosure may be administered continuously, along with an initial or periodic bolus injection.

The compounds of the disclosure may be prepared using synthetic methods as exemplified in the experimental section herein, as well as (where appropriate) standard procedures that are known to those skilled in the art of synthetic organic chemistry and used for the preparation of analogous compounds or moieties within compounds. Some appropriate synthetic procedures may be found, for example, in J. March, Advanced Organic Chemistry: Reactions, Mechanisms and Structure, 5th Edition (New York: Wiley-Interscience, 2001). Syntheses of representative compounds are detailed in the Examples provided herein; it will be appreciated that such synthetic methods are also within the scope of the invention.

In some embodiments, the compounds of the disclosure are synthesized by providing a core fragment having protease inhibitive properties and linking the core fragment (via, e.g., a coupling reaction) with an additional functional unit. The additional functional unit may be, for example, a protein binding moiety.

In some embodiments, the compounds of the disclosure are protease inhibitors. Accordingly, the compounds are capable of interfering with the activity of certain proteases, for example HIV protease. In some preferred embodiments, the compounds of the disclosure are equally effective at inhibiting HIV protease in cell free assays and in cell infectivity assays. That is, the presence of cellular matter does not reduce the efficacy of the compounds. In some other embodiments, the compounds of the disclosure exhibit a modest decrease in efficacy between a cell free assay and a cell infectivity assay. For example, the IC₅₀ values of the compounds in a cell infectivity assay are no more than 100% greater than the IC₅₀ values of the compounds in a cell free assay, or no more than 50% greater, or no more than 25% greater, or no more than 10% greater. In some embodiments, the IC₅₀ values of the compounds in a cell infectivity assay are less than the IC₅₀ values of the compounds in a cell free assay. In some preferred embodiments, the compounds of the disclosure exhibit IC₅₀ values in cell infectivity assays that are below about 75 nM, or below about 50 nM, or below about 25 nM, or below about 10 nM.

Accordingly, the compounds find utility in treating viral infections. In certain embodiments, the compounds are useful as inhibitors of HIV protease. The compounds of the disclosure, and compositions comprising such compounds, are useful in the treatment of AIDS or HIV infections, including multidrug-resistant strains of HIV. In certain embodiments, the compounds are also useful in treating other viral infections, such as Hepatitis C.

Accordingly, the disclosure provides a method for treating an HIV-infected patient, the method comprising administering to the patient an effective amount of any of the compounds disclosed herein. The disclosure also provides a method for preventing viral replication, the method comprising administering an effective amount of any of the compounds disclosed herein. The disclosure also provides a method for inhibiting the activity of HIV-1 protease, the method comprising administering an effective amount of any of the compounds disclosed herein. The disclosure also provides a method for treating a patient suffering from AIDS, the method comprising administering an effective amount of any of the compounds disclosed herein. The disclosure also provides a method for inhibiting the spread of HIV-virions to non-infected cells, the method comprising contacting a cell infected with HIV with an effective amount of any of the compounds disclosed herein. As described in more detail herein, in any of the aforementioned methods, the compound may be administered in a composition comprising one or more active agents and one or more additives.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description as well as the examples that follow, are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

### Example 1

### Preparation of (R)-tert-butyl 2-{3-[3-(3,4-dimethoxyphenyl)-1-hydroxypropyl]phenoxy}ethylcarbamate (6)

Compound 6 was prepared according to the method shown in Scheme 1.

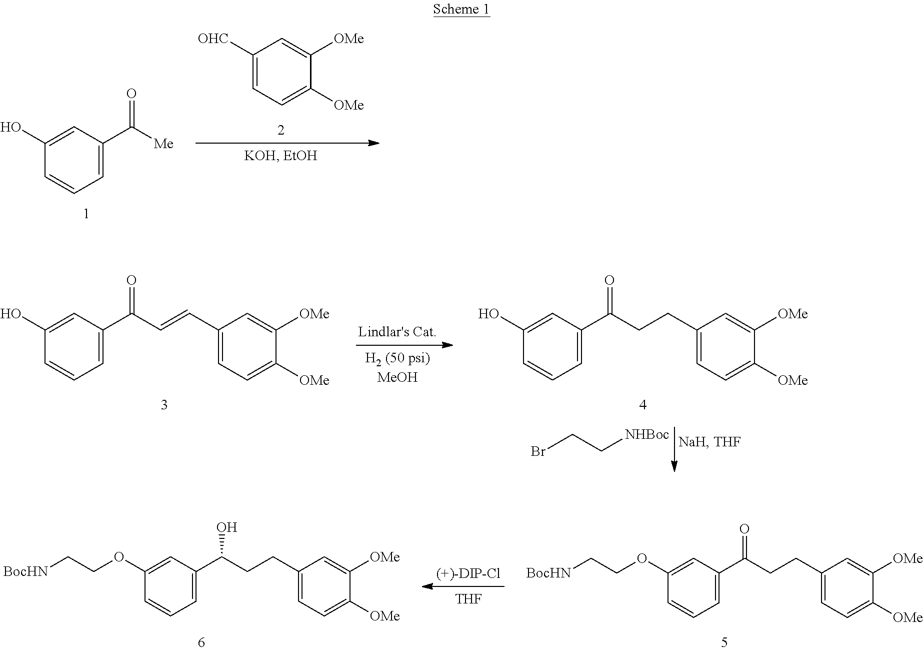

Preparation of (E)-3-(3,4-dimethoxyphenyl)-1-(3-hydroxyphenyl)prop-2-en-1-one (3): A cold solution of KOH (16.4 g, 293 mmol) in water (122 mL) was added to a stirred solution of ketone 1 (10.0 g, 73.4 mmol) and aldehyde 2 (12.2 g, 73.40 mmol) in ethanol (40 mL) at 0° C. and the reaction mixture was stirred overnight. The mixture was poured into ice water (122 mL) containing concentrated HCl (25 mL) and the aqueous layer was extracted with EtOAc (3×100 mL). The combined organic layers were washed with brine (1×100 mL), dried over anhydrous Na₂SO₄ and concentrated. The resultant solids were washed with CH₂Cl₂ and filtered to afford α,β-unsaturated ketone 3 (13.6 g, 65%) as a yellow solid: ¹H NMR (400 MHz, CDCl₃) δ 7.78 (d, J=15.6 Hz, 1H), 7.61 (s, 1H), 7.56 (d, J=7.6 Hz, 1H), 7.4-7.3 (m, 2H), 7.22 (d, J=6.4 Hz, 1H), 7.19-7.10 (m, 2H), 6.89 (d, J=6.4 Hz. 1H), 3.94 (s, 6H); MS (ESI) m/z 285 [C₁₇H₁₆O₄+H]⁺.

Preparation (E)-3-(3,4-dimethoxyphenyl)-1-(3-hydroxyphenyl)propane-1-one (4): Lindlar's catalyst (2.4 g) was added to a solution of ketone 3 (20 g, 70.20 mmol) in methanol (80 mL) and the reaction mixture was stirred in a parr apparatus under hydrogen (50 psi) for 6 h. The reaction mixture was filtered through celite, concentrated under reduced pressure, and the solids were washed with EtOAc to afford ketone 4 (13.5 g, 67%) as an off-white solid: ¹H NMR (400 MHz, DMSO-d₆) δ 9.74 (s, 1H), 7.42 (d, J=8.0 Hz, 1H), 7.31-7.28 (m, 2H), 7.01-6.98 (m, 1H), 6.87-6.81 (m, 2H), 6.75-6.70 (m, 1H), 3.71 (s, 3H), 3.69 (s, 3H), 3.26 (t, J=4.4 Hz, 2H), 2.85 (t, J=7.6 Hz, 2H).

Preparation of tert-butyl 2-{3-[3-(3,4-dimethoxyphenyl)propane]phenoxy}ethylcarbamate (5): A solution of phenol 4 (5.00 g, 17.45 mmol) in DMF (30 mL) was added to a suspension of NaH (60% in mineral oil, 800 mg, 20.2 mmol) in DMF (20 mL) at 0° C. and stirred for 0.25 h. To this mixture was added a solution of N-tert-butoxycarbonyl-2-bromo-ethanamine (5.00 g, 21.35 mmol) in DMF (20 mL). The reaction mixture was heated to 40° C. and stirred for 3 d at this temperature. The reaction mixture was cooled to 5° C., quenched with water (200 mL), and extracted with EtOAc (3×150 mL). The combined organic layers were washed with NaOH (1×150 mL), water (1×150 mL), and brine (1×150 mL), then dried over anhydrous Na₂SO₄, filtered and concentrated. As crude product (6.00 g) could not be purified by column chromatography, it was purified by deprotection of Boc group to amine salt by addition of 20% HCl in dioxane (25 mL) at 0° C. and stirred for 2 h. Then it was concentrated and triturated with EtOAc (25 mL) to afford amine salt (4.10 g). Then the amine salt was again protected with Boc by addition of (Boc)₂O (2.68 g, 12.27 mmol) and aqueous sat. NaHCO₃ (10 mL) to the amine salt in dioxane (10 mL) at 0° C. and stirred for 2 h. It was then extracted with EtOAc (3×100 mL), washed with brine (1×100 mL), then dried over anhydrous Na₂SO₄, filtered and concentrated. The residue was purified by column chromatography (20% EtOAc in petroleum ether) to afford the protected amine 5 (3.90 g, 52%) as yellow oil. ¹H NMR (400 MHz, CDCl₃) δ 7.54 (d, J=7.6 Hz, 1H), 7.47 (d, J=2.4 Hz, 1H), 7.38-7.34 (t, J=8.0 Hz, 1H), 7.09 (dd, J=2.0, 8.0 Hz, 1H), 6.82-6.77 (m, 3H), 5.01 (bs, 1H), 4.07-4.04 (m, 2H), 3.87 (s, 3H), 3.86 (s, 3H), 3.55 (bs, 2H), 3.28-3.24 (m, 2H), 3.09-2.99 (m, 2H), 1.45 (s, 9H); MS (ESI) m/z 429.22 [C₂₄H₃₁NO₆+H]⁺.

Preparation of (R)-tert-butyl 2-{3-[3-(3,4-dimethoxyphenyl)-1-hydroxypropyl]phenoxy}ethylcarbamate (6): A solution of (+)-DIP-chloride (7.82 g, 24.4 mmol) in THF (9 mL) was added a solution of ketone 5 (7.00 g, 16.29 mmol) in THF (18 mL) at −20° C. The resulting mixture was allowed to stand at −10° C. for 24 h and then the solvent was removed under reduced pressure. The residue was treated with Et₂O (55 mL) followed by diethanolamine (14.0 mL, 146.67 mmol) and was stirred for 12 h. This mixture was diluted with EtOAc (150 mL) and filtered through celite. The filtrate was concentrated and the residue was purified by silica-gel chromatography (25% EtOAc in petroleum ether) to afford alcohol 6 (3.80 g, 55%) as a light brown oil: ¹H NMR (400 MHz, CDCl₃) δ 7.29-7.25 (m, 2H), 6.94-6.91 (m, 2H), 6.82-6.72 (m, 3H), 4.68-4.65 (m, 1H), 4.03-4.01 (m, 2H), 3.87 (s, 3H), 3.86 (s, 3H), 5.00 (bs, 1H), 3.54-3.51 (m, 2H), 2.71-2.63 (m, 2H), 2.32-2.27 (m, 1H), 1.40 (s, 9H), 1.40-1.20 (m, 2H).

### Example 2

### Preparation of (2S)-1-(1,2-dioxo-3,3-dimethylpentyl)-2-piperidinecarboxylic acid (10)

Compound 10 was prepared according to the method shown in Scheme 2.

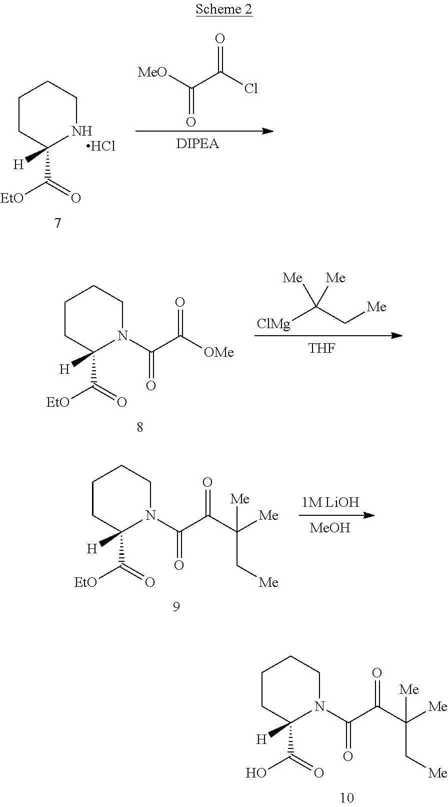

Preparation of Ethyl (2S)-1-(1,2-dioxo-2-methoxyethyl)-2-piperidinecarboxylate (8): To a stirred solution of ethyl piperidine-2-carboxylate (7, 20.0 g, 103.3 mmol) in CH₂Cl₂ (30 mL) at 0° C. was added N,N-diisopropylethylamine (45.0 mL, 258.2 mmol) and methyl chlorooxoacetate (11.9 mL, 129.1 mmol). The reaction mixture was stirred at rt for 1.5 h, then diluted with CH₂Cl₂ (250 mL) and washed with water (300 mL) and brine (200 mL), dried over Na₂SO₄, filtered and concentrated. The residue was purified by column chromatography (20% EtOAc in petroleum ether) to afford amide 8 (17.5 g, 69%) as a reddish oil: ¹H NMR (CDCl₃, 400 MHz, δ 5.00 (d, J=5.2 Hz, 1H), 4.13 (q, J=14.4 Hz, 2H), 3.80 (s, 3H), 3.41 (br d, J=13.2 Hz, 1H), 3.20-3.17 (m, 1H), 2.15 (br d, J=14.4 Hz, 1H), 1.69-1.61 (m, 4H), 1.39-1.27 (m, 4H).

Preparation of Ethyl (2S)-1-(1,2-dioxo-3,3-dimethylpentyl)-2-piperidinecarboxylate (9): To a solution of diester 8 (18.50 g, 76.05 mmol) in THF (153 mL) at −78° C. was added 1,1-dimethylpropylmagnesium chloride (1M in Et₂O, 101.4 mL, 101.4 mmol) under argon atmosphere and the reaction mixture was stirred for 3 h at −78° C. The reaction mixture was then poured in to saturated aqueous NH₄Cl (150 mL) and the aqueous layer was extracted with EtOAc (2×250 mL). The combined organic layers were washed with brine (200 mL), dried over Na₂SO₄, filtered and concentrated. The crude product was purified by column chromatography (5% EtOAc in petroleum ether) to afford ketone 9 (16.1 g, 75%) as a light yellow oil: ¹H NMR (CDCl₃, 400 MHz) δ 5.25 (d, J=5.6 Hz, 1H), 4.23 (q, J=13.0 Hz, 2H), 3.41 (br d, J=13.2 Hz, 1H), 3.22 (td, J=12.8 Hz, 3.2 Hz, 1H), 2.35 (br d, J=13.6 Hz, 1H), 1.79-1.31 (m, 7H), 1.29 (t, J=7.2 Hz, 3H), 1.23 (s, 3H), 1.19 (s, 3H), 0.91-0.82 (m, 3H).

Preparation of (2S)-1-(1,2-dioxo-3,3-dimethylpentyl)-2-piperidinecarboxylic acid (10): A mixture of ketone 9 (16.1 g, 56.87 mmol) and LiOH (1.0N in water, 86.6 mL) in methanol (500 mL) was stirred at 0° C. for 0.5 h and then warmed to rt and stirred at that temperature for 16 h. The reaction mixture was acidified with HCl (10% in water, 50 mL), and extracted with CH₂Cl₂ (3×500 mL). The combined organic layers were washed with water (250 ml) and brine (300 mL), dried over Na₂SO₄, filtered and concentrated to afford an off-white solid which was further purified by trituration with hexane to afford acid 10 (10.6 g, 73%) as a white solid: ¹H NMR (CDCl₃, 400 MHz) δ 5.32 (d, J=4.4 Hz, 1H), 3.41 (br d, J=14.0 Hz, 1H), 3.25 (br t, J=13.2 Hz, 1H), 2.35 (br d, J=13.6 Hz, 1H), 1.84-1.43 (m, 7H), 1.25 (s, 3H), 1.20 (s, 3H), 0.91-0.82 (m, 3H).

### Example 3

### Preparation of (1R)-3-(3,4-di-methoxyphenyl)-1-[3-(2-ethanamine)phenoxyl]-1-propanyl (2S)-1-(3,3-dimethyl-1,2-dioxopentyl)-2-piperidinecarboxylate hydrochloride (12)

Compound 12 was prepared according to the method shown in Scheme 3.

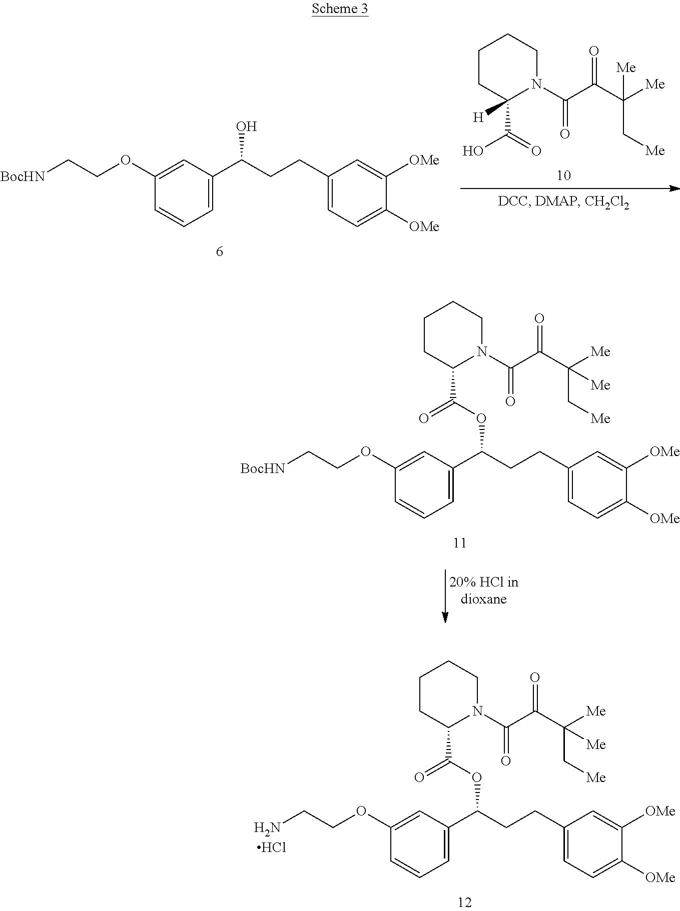

Preparation of (S)-{(R)-1-{3-[2-(tert-butoxycarbonylamino)ethoxy]phenyl}-3-[3,4-dimethoxyphenyl]propyl}-1-(3,3-dimethyl-2-oxopentanoyl)piperidine-2-carboxylate (11) IN-YSA-A-178: To a stirred solution of alcohol 6 (3.80 g, 8.81 mmol) and acid 10 (2.44 g, 9.69 mmol) in CH₂Cl₂ (50 mL) at 0° C. was added DMAP (108 mg, 0.881 mmol) followed by DCC (1.99 g, 9.69 mmol) and stirred for 2 h. The reaction mixture was diluted with EtOAc (100 mL) and filtered through celite. The solvent was concentrated to dryness and the crude residue was purified by column chromatography (20% EtOAc in petroleum ether) to afford ester 11 (4.12 g, 70%) as a yellow oil: ¹H NMR (400 MHz, CDCl₃) δ 7.29-7.25 (m, 2H), 6.90-6.77 (m, 3H), 6.71-6.69 (m, 2H), 5.78-5.76 (m, 1H), 5.32-5.29 (m, 1H), 5.01 (bs, 1H), 4.04-4.02 (m, 2H), 3.86 (s, 3H), 3.85 (s, 3H), 3.55 (bs, 2H), 3.35-3.32 (m, 1H), 3.17-3.14 (m, 1H), 2.57-2.55 (m, 2H), 2.38-2.35 (m, 1H), 2.31-2.18 (m, 1H), 1.77-1.45 (m, 7H), 1.45 (s, 9H), 1.23 (s, 3H), 1.21 (s, 3H), 0.95 (t, 3H); MS (ESI) m/z 669.3 [C₃₇H₅₂N₂O₉+H]⁺.

Preparation of (1R)-3-(3,4-di-methoxyphenyl)-1-[3-(2-ethanamine)phenoxyl]-1-propanyl (2S)-1-(3,3-dimethyl-1,2-dioxopentyl)-2-piperidinecarboxylate hydrochloride (12, SLFA2); IN-YSA-A-179: To a stirred solution of Boc-protected amine 11 (4.10 g, 6.13 mmol) in CH₂Cl₂ (5 mL) at 0° C. was added 20% HCl in 1,4-dioxane (11.1 mL, 60.13 mmol) and reaction mixture was allowed to stir at rt for 16 h. The solvent was concentrated to afford amine 9 (SLFA2, 4.00 g, >99%) as an off-white foam: ¹H NMR (400 MHz, CDCl₃) δ 8.55 (bs, 1H), 7.30-7.15 (m, 1H), 6.93-6.68 (m, 7H), 5.80-5.70 (m, 1H), 5.35-5.30 (m, 1H), 4.30-4.18 (m, 2H), 3.86 (s, 3H), 3.84 (s, 3H), 3.40-3.30 (m, 2H), 3.16-3.14 (m, 1H), 2.38-2.26 (m, 3H), 2.05 (bs, 1H), 1.74-1.60 (m, 5H), 1.53-1.30 (m, 2H), 1.25 (s, 3H), 1.23 (s, 3H), 0.9 (m, 3H); MS (ESI) m/z 569.3 [C₃₂H₄₄N₂O₇+H]⁺.

### Example 4

### Preparation of 2-(3-((R)-3-(3,4-dimethoxyphenyl)-1-(S)-1-(3,3-dimethyl-2-oxopentanoyl)piperidine-2-carbonyloxy)propyl)phenoxy)acetic acid (16)

Compound 16 was prepared according to the method shown in Scheme 4.

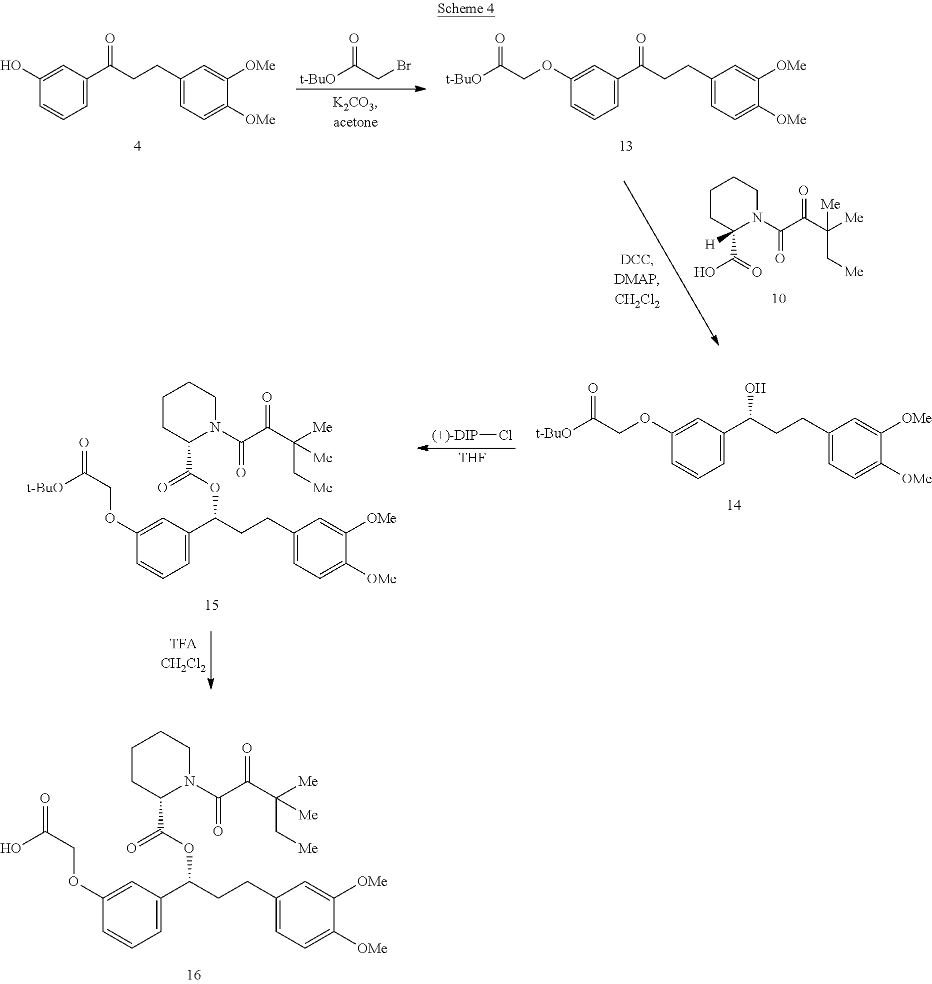

Preparation of tert-butyl 2-[3-(3,4-dimethoxyphenyl)propanoyl]phenoxyacetate (13): A slurry of phenol 4 (10.0 g, 34.9 mmol) and K₂CO₃ (9.66 g, 69.9 mmol) in acetone (100 mL) was treated with tert-butyl bromoacetate (5.67 mL, 38.4 mmol) at rt for 24 h. The reaction mixture was filtered and the solvent was concentrated. The residue was purified by column chromatography (20% EtOAc in petroleum ether) to afford ether 13 (12.4 g, 88%) as a light yellow oil: ¹H NMR (400 MHz, CDCl₃) δ 7.60 (d, J=8.0 Hz, 1H), 7.48 (s, 1H), 7.42-7.37 (m, 1H), 7.14-7.11 (m, 1H), 6.82-6.77 (m, 3H), 4.57 (s, 2H), 3.87 (s, 3H), 3.86 (s, 3H), 3.28 (t, J=6.8 Hz, 2H), 3.03 (t, J=7.2 Hz, 2H), 1.49 (s, 9H).

Preparation of (R)-tert-butyl 2-[3-(3,4-dimethoxyphenyl)-1-hydroxypropyl]phenoxyacetate (14): (+)-DIP-chloride (3.63 g, 11.33 mmol) in THF (9 mL) was added to a solution of ketone 13 (2.40 g, 7.55 mmol) in THF (6 mL) at −20° C. The resultant solution was allowed to stand at −10° C. for 16 h and the solvent was removed under reduced pressure. The residue was diluted with Et₂O (25 mL) followed by addition of diethanolamine (6.51 mL, 68.0 mmol) and stirred for 6 h. This mixture was diluted with ethyl acetate (50 mL), filtered through celite, and the filtrate was concentrated. The crude product was purified by column chromatography (25% EtOAc in petroleum ether) to afford alcohol 14 (1.42 g, 58%) as a light brown oil: ¹H NMR (400 MHz, CDCl₃) δ 7.29-7.22 (m, 1H), 6.95-6.90 (m, 2H), 6.82-6.78 (m, 2H), 6.71 (d, J=1.6 Hz, 2H), 4.68-4.65 (m, 1H), 4.52 (s, 2H), 3.87 (s, 3H), 3.86 (s, 3H), 2.75-2.58 (m, 2H), 2.35-2.30 (m, 1H), 2.10-1.95 (m, 3H), 1.49 (s, 9H).

Preparation of (S)-{(R)-1-[3-(2-tert-butoxy-2-oxoethoxy)phenyl]-3-(3,4-dimethoxyphenyl)propyl-(3,3-dimethyl-2-oxopentanoyl)}piperidine-2-carboxylate (15): To a solution of alcohol 14 (1.00 g, 2.68 mmol) and acid 10 (750 mg, 2.95 mmol) in CH₂Cl₂ (10 mL) at 0° C. was added DMAP (30 mg, 0.29 mmol) and DCC (600 mg, 2.95 mmol). The reaction mixture was stirred for 2 h, diluted with EtOAc (40 mL) and filtered through celite. The filtrate was concentrated and the crude residue was purified column chromatography (20% EtOAc in petroleum ether) to afford ester 15 (1.30 g, 72%) as a light yellow oil: ¹H NMR (400 MHz, CDCl₃) δ 7.25-7.17 (m, 2H), 6.95-6.70 (m, 5H), 5.75-5.67 (m, 1H), 5.25 (bs, 1H), 4.51 (s, 1H), 3.86 (s, 3H), 3.85 (s, 3H), 3.39-3.30 (m, 1H), 3.19-3.10 (m, 1H), 2.60-2.49 (m, 2H), 2.39-2.20 (m, 2H), 2.05-2.01 (m, 1H), 1.78-1.55 (m, 5H), 1.47 (s, 9H), 1.24 (s, 3H), 1.23 (s, 3H), 0.88 (t, 3H).

Preparation of 2-(3-((R)-3-(3,4-dimethoxyphenyl)-1-(S)-1-(3,3-dimethyl-2-oxopentanoyl)piperidine-2-carbonyloxy)propyl)phenoxy)acetic acid (16): To a solution of tert-butylester 15 (1.00 g, 2.03 mmol) in CH₂Cl₂ (30 mL) at 0° C. was added TFA (10.1 mL) and the solution was stirred at rt for 2 h. The reaction mixture was diluted with toluene (20 mL) and solvents were removed under reduced pressure. The crude product was purified by column chromatography (2% MeOH in CH₂Cl₂) to afford acid 16 (850 mg, 92%) as a yellow foam: ¹H NMR (400 MHz, CDCl₃) δ 7.40-7.31 (m, 1H), 6.94-6.88 (m, 3H), 6.80 (d, J=6.0 Hz, 1H), 6.70 (d, J=8.4 Hz, 2H), 5.76-5.70 (m, 1H), 5.32 (d, J=6.8 Hz, 1H), 4.70 (s, 2H), 3.87 (s, 3H), 3.86 (s, 3H), 3.41-3.19 (m, 4H), 2.70-2.54 (m, 2H), 2.45-2.05 (m, 3H), 1.85-1.65 (m, 5H), 1.22-1.14 (m, 8H), 0.87 (t, J=7.8 Hz, 3H).

### Example 5

### Preparation of N-[(2R,3S)-3-amino-2-hydroxy-4-phenylbutyl]-N-isobutyl-4-nitrobenzenesulfonamide hydrochloride (20)

Compound 20 was prepared according to the method shown in Scheme 5.

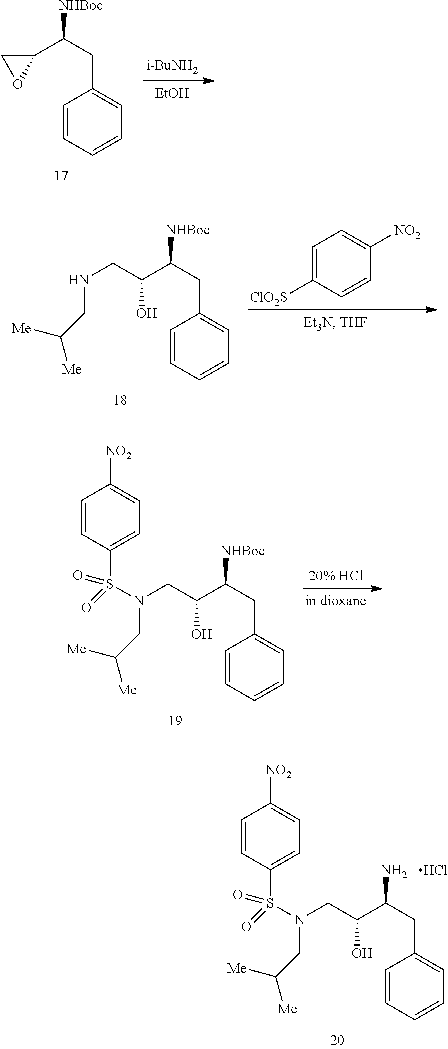

Preparation of tert-butyl (2S,3R)-3-hydroxy-4-(isobutylamino)-1-phenylbutan-2-ylcarbamate (18): To a stirred solution of epoxide 17 (5.00 g, 18.98 mmol) in ethanol (140 ml) was added isobutylamine (19.0 ml, 189.9 mmol) and the reaction mixture was heated to 80° C. and stirred for 3 h at that temperature. Upon cooling to rt, the solvents were concentrated off and the residue was triturated with hexanes (3×30 mL) to afford amino alcohol 18 (5.43 g, 85%) as a white solid: ¹H NMR (400 MHz, CDCl₃): δ 7.31-7.21 (m, 5H), 4.73-4.70 (m, 1H), 3.81 (bs, 1H), 3.48-3.46 (m, 1H), 3.01-2.96 (m, 1H), 2.88-2.86 (m, 1H), 2.70-2.68 (m, 2H), 2.42 (m, 2H), 1.75-1.60 (m, 2H), 1.44 (s, 9H), 0.92 (d, J=5.0 Hz, 2H); MS (ESI) m/z 336 [C₁₉H₃₂N₂O₃+H]⁺.

Preparation of tert-butyl (2S,3R)-3-hydroxy-4-(N-isobutyl-4-nitrophenylsulfonamido)-1-phenylbutan-2-ylcarbamate (19): Triethylamine (2.47 mL, 17.75 mmol) was added to a solution of amine 18 (5.43 g, 16.13 mmol) in CH₂Cl₂ (25 mL) at 0° C. To this mixture was added 4-nitrobenzenesulfonylchloride (3.75 g, 16.94 mmol) and the reaction was stirred at ambient temperature for 3 h. The reaction mixture was washed with water (2×20 mL), dried over Na₂SO₄, filtered and concentrated. The crude compound was purified by column chromatography (1.5% MeOH in CH₂Cl₂) to afford sulfonamide 19 (6.73 g, 80%) as a pale yellow solid: ¹H NMR (400 MHz, DMSO-d₆) δ 8.36 (d, J=8.8 Hz, 2H), 8.05 (d, J=8.8 Hz, 2H), 7.24-7.13 (m, 5H), 6.68 (d, J=8.8 Hz, 1H), 4.95 (d, J=6.4 Hz, 1H), 4.33 (d, J=4.4 Hz, 1H), 3.78-3.74 (m, 1H), 3.49-3.33 (m, 3H), 3.17-3.3.03 (m, 2H), 2.95-2.90 (m, 2H), 1.23 (s, 9H), 0.93-0.90 (m, 6H); MS (ESI) m/z 521 [C₂₅H₃₅N₃O₇S+H]⁺.

Preparation of N-[(2R,3S)-3-amino-2-hydroxy-4-phenylbutyl]-N-isobutyl-4-nitrobenzenesulfonamide hydrochloride (20, PI-1.4): A solution of 20% HCl in 1,4-dioxane (35.3 mL, 19.4 mmol) was added to carbamate 19 (6.73 g, 12.90 mmol) at 0° C. The reaction was stirred at rt for 1 h and the solvent was concentrated. The residue was dissolved in CH₂Cl₂ (40 mL) and evaporated (the same process was repeated four times) to provide pure amine hydrochloride 20 (5.02 g, 85%) as a white solid: ¹H NMR (400 MHz, DMSO-d₆) δ 8.37 (d, J=8.8 Hz, 2H), 8.06 (d, J=8.8 Hz, 2H), 7.36-7.25 (m, 5H), 5.63 (d, J=6.0 Hz, 1H), 3.94-3.91 (m, 1H), 3.44-3.39 (m, 2H), 3.06-2.82 (m, 3H), 1.92-1.85 (m, 1H), 0.80 (d, J=6.8 Hz, 3H), 0.71 (d, J=6.4 Hz, 3H); MS (ESI) m/z 421 [C₂₀H₂₇N₃O₅S+H]⁺.

### Example 6

### Preparation of (S)—{(R)-1-[3-{(10S,11R)-13-(4-aminophenylsulfonyl)-10-benzyl-11-hydroxy-15-methyl-2,8-dioxo-7-oxa-3,9,13-triazahexadecyloxy}phenyl]-3-(3,4-dimethoxyphenyl)propyl}1-(3,3-dimethyl-2-oxopentanoyl)piperidine-2-carboxylate (23)

Compound 23 was prepared according to the method shown in Scheme 6.

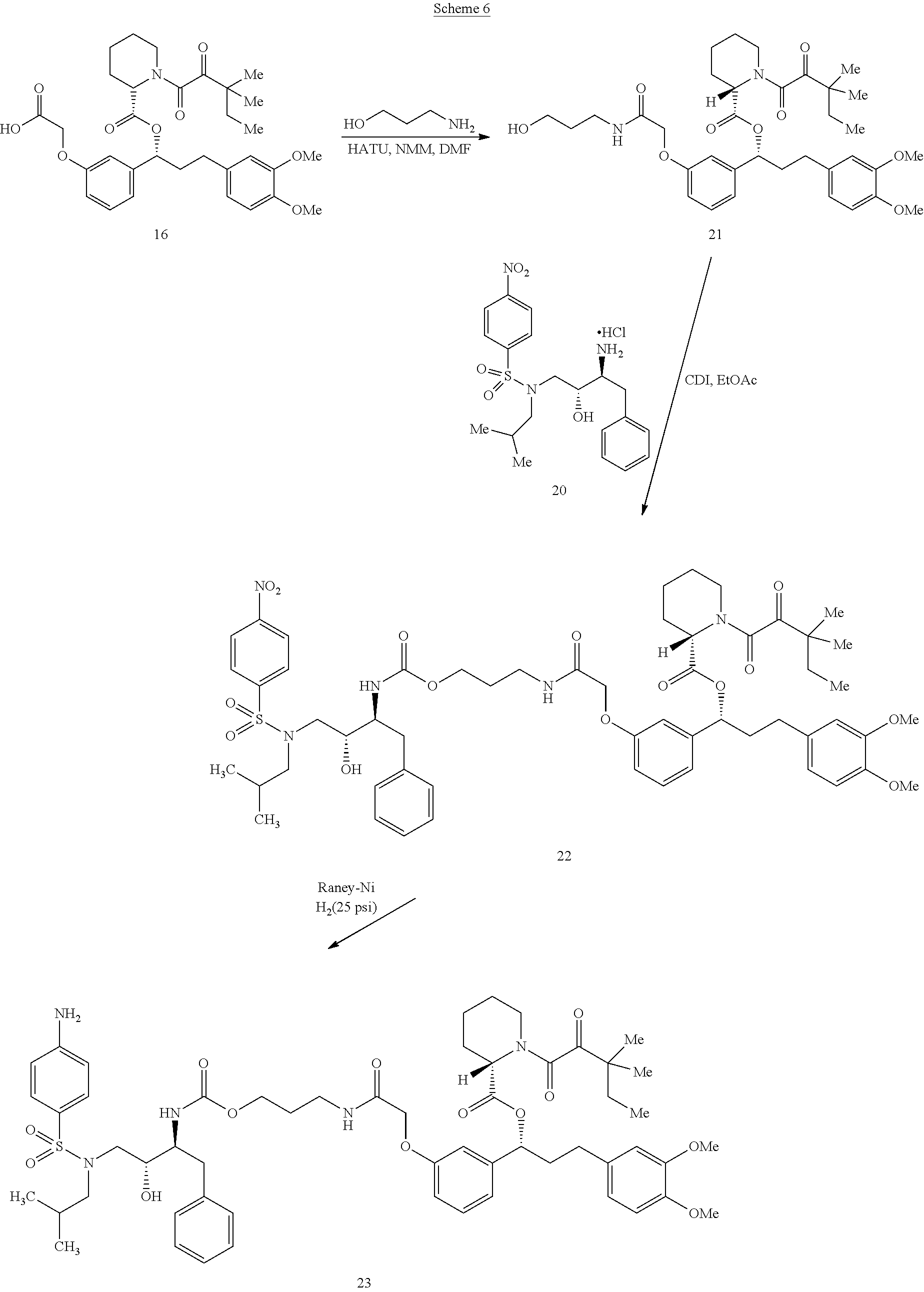

Preparation of (S)-{(R)-3-[3,4-dimethoxyphenyl]-1-{3-[2-(3-hydroxypropylamino)-2-oxoethoxy]phenyl}propyl}-1-(3,3-dimethyl-2-oxopentanoyl)piperidine-2-carboxylate (21): To a solution of acid 16 (750 mg, 1.28 mmol) in DMF (10 mL) was added HATU (480 mg, 1.28 mmol) and NMM (140 μL, 1.28 mmol) followed by addition of 3-amino propanol (90 μL, 1.28 mmol). The reaction was heated to 80° C. for 3 h and poured onto water (30 mL). The aqueous layer was extracted with EtOAc (3×25 mL) and the combined organic layers were washed with brine (50 mL), dried over anhydrous Na₂SO₄, filtered and concentrated. The crude product was purified by column chromatography (25% EtOAc in petroleum ether) to afford amide 21 (670 mg, 82%) as a yellow oil: ¹H NMR (400 MHz, CDCl₃) δ 8.01 (s, 1H), 7.30-7.28 (m, 1H), 6.97-6.95 (m, 2H), 6.83-6.77 (m, 2H), 6.69-6.68 (m, 2H), 5.76-5.70 (m, 1H), 5.30 (s, 1H), 4.52 (s, 2H), 3.86 (s, 3H), 3.85 (s, 3H), 3.66 (t, J=5.2 Hz, 2H), 3.52 (t, J=6.0 Hz, 2H), 3.41-3.19 (m, 2H), 2.70-2.54 (m, 2H), 2.45-2.05 (m, 3H), 1.85-1.65 (m, 5H), 1.22-1.14 (m, 8 H), 0.90-0.85 (m, 3H).

Preparation of (S)-{(R)-1-{3-[(10S,11R)-10-benzyl-11-hydroxy-15-methyl-13-(4-nitrophenylsulfonyl)-2,8-dioxo-7-oxa-3,9,13-triazahexadecyloxy]-phenyl}-3-(3,4-dimethoxyphenyl)propyl}-1-(3,3-dimethyl-2-oxopentanoyl)piperidine-2-carboxylate (22): To a solution of alcohol 21 (100 mg, 0.15 mmol) in EtOAc (5 mL) was added CDI (20 mg, 0.15 mmol) and the reaction was stirred for 2 h. To this mixture was further added amine hydrochloride 20 (70 mg, 0.15 mmol) and the reaction was refluxed for 22 h. The reaction was cooled to rt and the solvents were removed concentrated. The crude residue was purified by preparative TLC (3% MeOH in CH₂Cl₂) to afford conjugate 22 (10 mg, 7%) as a light yellow foam: ¹H NMR (400 MHz, CDCl₃) δ 8.36 (d, J=8.8 Hz, 2H), 8.04 (d, J=8.8 Hz), 7.30-6.70 (m, 12H), 5.71-5.60 (m, 1H), 5.22-5.12 (m, 1H), 5.00 (d, J=6.4 Hz, 1H), 4.45 (s, 2H), 3.81-3.80 (m, 1H), 3.70 (s, 3H), 3.69 (s, 3H), 3.52-3.50 (m, 2H), 3.13-3.08 (m, 4H), 2.93-2.89 (m, 1H), 1.75-1.51 (m, 7H), 1.25-1.14 (m, 9H), 0.84-0.76 (m, 6H).

Preparation of (S)-{(R)-1-[3-{(10S,11R)-13-(4-aminophenylsulfonyl)-10-benzyl-11-hydroxy-15-methyl-2,8-dioxo-7-oxa-3,9,13-triazahexadecyloxy}phenyl]-3-(3,4-dimethoxyphenyl)propyl}1-(3,3-dimethyl-2-oxopentanoyl)piperidine-2-carboxylate (23): Raney-Ni (50% in water, 500 mg) was added to a solution of nitro 22 (200 mg, 0.12 mmol) in ethanol (10 mL) and the reaction mixture was stirred in a parr apparatus under hydrogen (25 psi) for 4 h. The reaction mixture was filtered through celite and concentrated under reduced pressure. The crude product was purified by preparative TLC (3% MeOH in CH₂Cl₂) to afford aniline 23 (50 mg, 40%) as a yellow white solid: ¹H NMR (400 MHz, MeOD) δ 7.47 (d, J=8.8 Hz, 2H), 7.20-6.70 (m, 12H), 5.80-5.70 (m, 1H), 4.51 (s, 2H), 3.80 (s, 3H), 3.79 (s, 3H), 3.16-3.12 (m, 2H), 3.05-2.95 (m, 1H), 2.90-2.75 (m, 2H), 2.70-2.55 (m, 3H), 2.45-1.95 (m, 4H), 1.73-1.65 (m, 5H), 0.92-0.85 (m, 6H); MS (APCI) m/z 1059 [C₅₆H₇₅N₅O₁₃S+H]⁺.

### Example 7

### Preparation of (S)-{(R)-1-(3-[(11S,12R)-11-benzyl-12-hydroxy-16-methyl-14-(4-nitrophenylsulfonyl)-4,9-dioxo-8-oxa-3,10,14-triazaheptadecyloxy]phenyl-3-(3,4-dimethoxyphenyl)propyl}-1-(3,3-dimethyl-2-oxopentanoyl)piperidine-2-carboxylate (27)

Compound 27 was prepared according to the method shown in Scheme 7

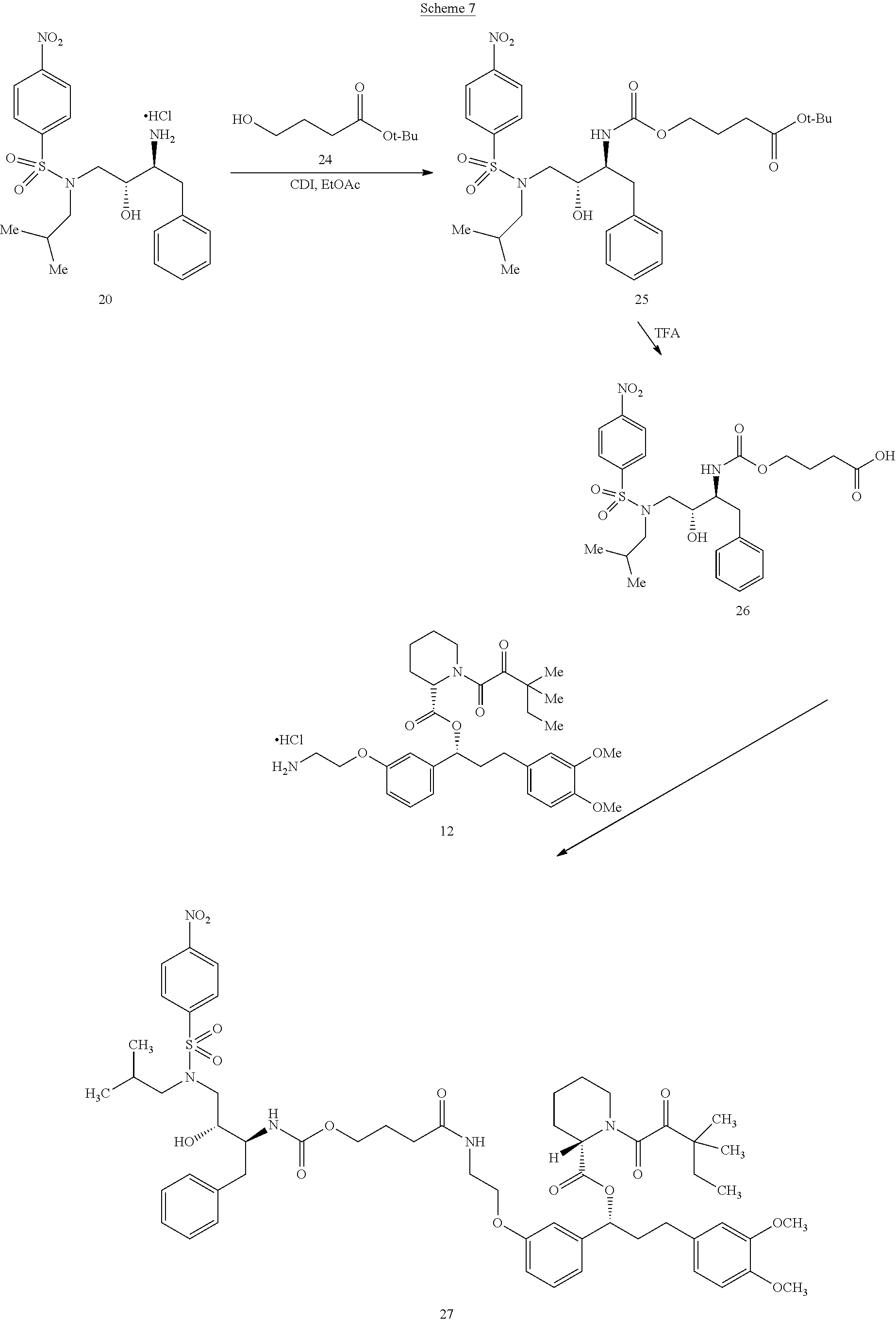

Preparation of tert-butyl-4-[(2S,3R)-3-hydroxy-4-(N-isobutyl-4-nitrophenylsulfonamido)-1-phenylbutan-2-ylcarbamoyloxy]butane (25): To a solution of tert-butyl-4-hydroxybutyrate (24, 100 mg, 0.62 mmol) in EtOAc (5 mL) was added CDI (101 mg, 0.62 mmol) and the reaction mixture was stirred for 3 h. Amine hydrochloride 20 (285 mg, 0.62 mmol) was added to the reaction mixture and the reaction was refluxed for 22 h. Upon cooling to rt the solvent was evaporated and the resultant residue was purified by column chromatography (silica-gel, gradient 10-20% EtOAc in petroleum ether) to afford impure tert-butylester 25 (140 mg) as yellow oil: ¹H NMR (CDCl₃, 400 MHz) δ 8.36 (d, J=8.8 Hz, 2H), 7.96 (d, J=8.8 Hz, 2H), 7.34-7.22 (m, 5H), 4.77-4.76 (m, 1H), 4.03-3.97 (m, 2H), 3.85-3.82 (m, 1H), 3.20-3.17 (m, 2H), 3.02-2.87 (m, 2H), 2.38-2.34 (m, 2H), 2.24-2.18 (m, 2H), 1.89-1.82 (m, 3H), 1.46 (s, 9H), 0.91-0.87 (m, 6H).

Preparation of 4-[(2S,3R)-3-hydroxy-4-(N-isobutylnitrophenylsulfonamido)-1-phenylbutan-2-ylcarbamoyloxy]butanoicacid (26): Trifluoroacetic acid (1.15 mL) was added to a solution of the above impure tert-butylester 25 (140 mg) in CH₂Cl₂ (4.0 mL) at 0° C. The reaction mixture was allowed to warm to rt and react for 2 h. The mixture was diluted with toluene (10 mL) and solvents were evaporated under reduced pressure to afford acid 26 (65 mg, 55%) as an off white solid which was directly used for the next step without purification.

Preparation of (S)-{(R)-1-(3-[(11S,12R)-11-benzyl-12-hydroxy-16-methyl-14-(4-nitrophenylsulfonyl)-4,9-dioxo-8-oxa-3,10,14-triazaheptadecyloxy]phenyl-3-(3,4-dimethoxyphenyl)propyl}-1-(3,3-dimethyl-2-oxopentanoyl)piperidine-2-carboxylate (27): To a solution of acid 26 (65 mg, 0.11 mmol) in DMF (4 mL) was added HATU (44 mg, 0.11 mmol) and N-methylmorpholine (12.0 μL, 0.11 mmol) followed by addition of amine 12 (71 mg, 0.11 mmol). The reaction mixture was heated at 80° C. for 4 h, cooled to rt and diluted with EtOAc (15 mL). The resulting solution was washed with water (3×10 mL) and brine (2×10 mL), dried over anhydrous Na₂SO₄, filtered and concentrated. The crude product was purified by column chromatography (silica-gel, gradient CH₂Cl₂ to 1% MeOH/CH₂Cl₂). Further purification by preparative TLC (1% MeOH/CH₂Cl₂) provided amide 27 (30 mg, 23%) as a light yellow foam which had 91% purity as observed by HPLC analysis: ¹H NMR (400 MHz, CDCl₃) δ 8.33 (d, J=8.40 Hz, 2H), 7.96 (d, J=8.40 Hz, 2H), 7.29-7.16 (m, 6H), 6.95-6.90 (m, 2H), 6.95-6.90 (m, 2H), 6.86-6.84 (m, 2H), 6.70-6.66 (m, 2H), 6.12-6.10 (m, 1H), 5.79-5.75 (m, 1H), 5.31-5.30 (m, 1H), 4.85-4.90 (m, 1H), 4.10-3.95 (m, 4H), 3.86 (s, 3H), 3.85 (s, 3H), 3.79-3.74 (m, 1H), 3.65-3.62 (m, 2H), 3.38-3.35 (m, 1H), 3.22-3.20 (m, 2H), 3.01-2.97 (m, 2H), 2.83-2.81 (m, 1H), 2.62-2.53 (m, 2H), 2.39-2.32 (m, 1H), 2.29-2.27 (m, 2H), 2.25-2.16 (m, 2H), 2.14-2.05 (m, 1H), 1.92-1.69 (m, 5H), 1.68-1.36 (m, 8H), 1.40-1.10 (m, 6H), 0.90-0.79 (m, 6H); MS (ESI) m/z 1101.7 [C₅₇H₇₅N₅O₁₅S]⁺.

### Example 8

### Synthesis of A1.4

Compound A1.4 is prepared according to the method shown in Scheme 8.

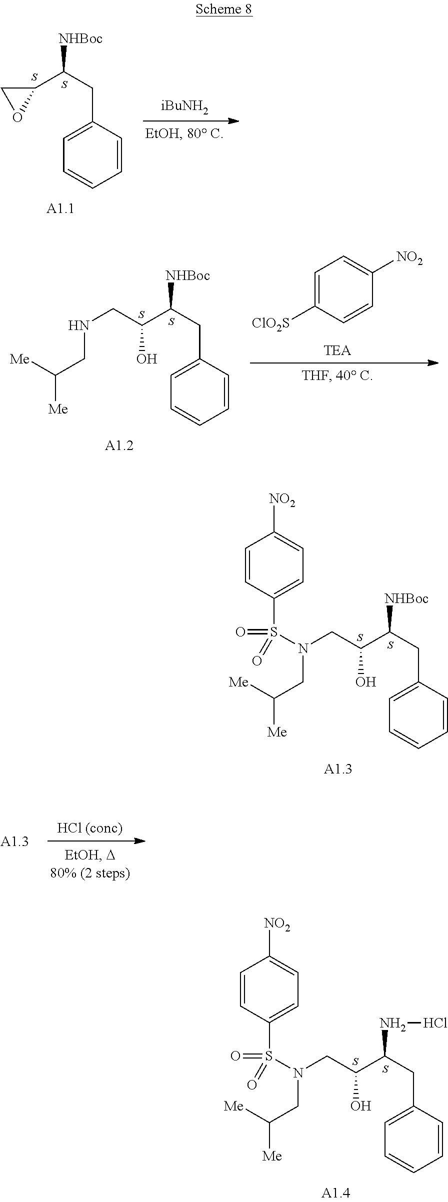

### Example 9a

### Synthesis of B1

Compound B1 is prepared according to the method shown in Scheme 9a.

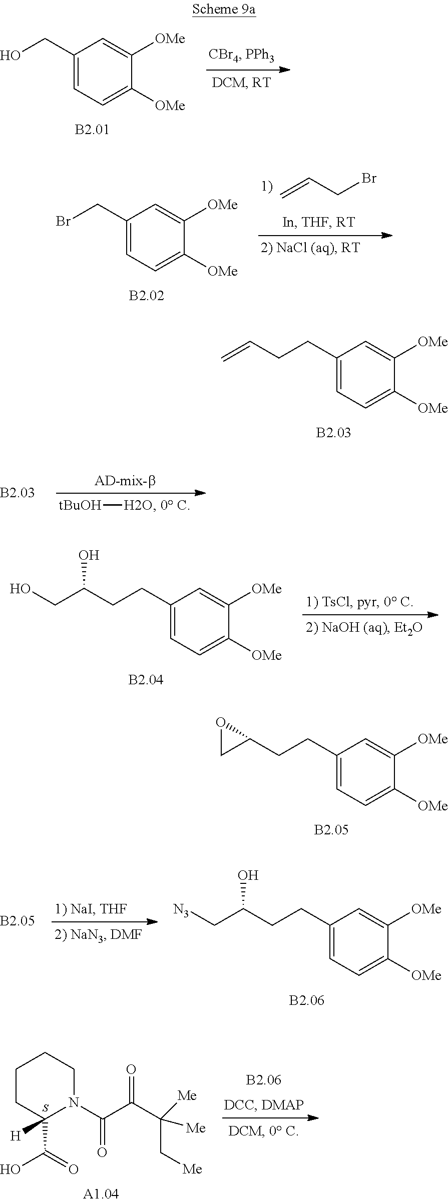

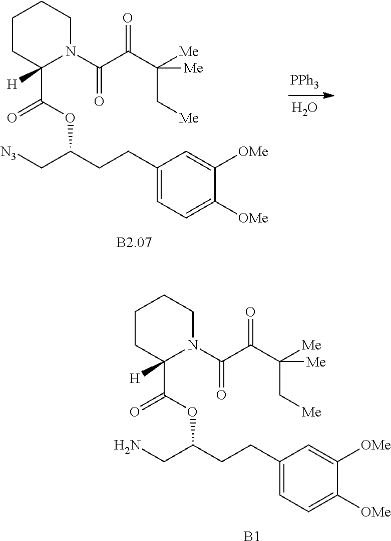

### Example 9b

### Synthesis of B2

Compound B2 is prepared according to the method shown in Scheme 9b.

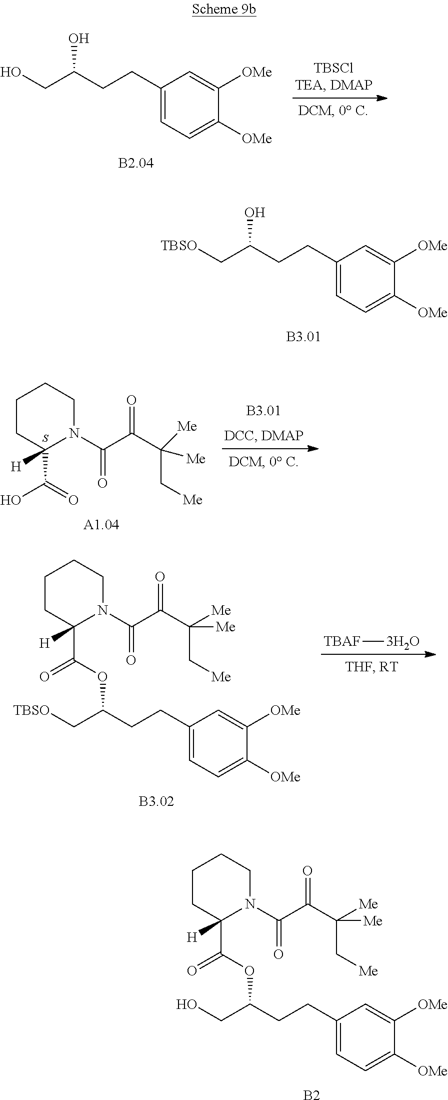

### Example 10a

### Synthesis of C1

Compound C1 is prepared according to the method shown in Scheme 10a.

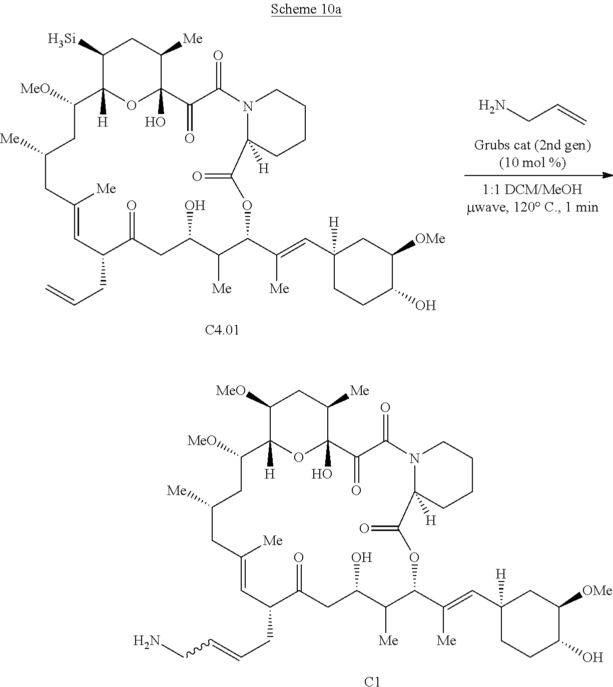

### Example 10b

### Synthesis of D1

Compound D1 is prepared according to the method shown in Scheme 10b.

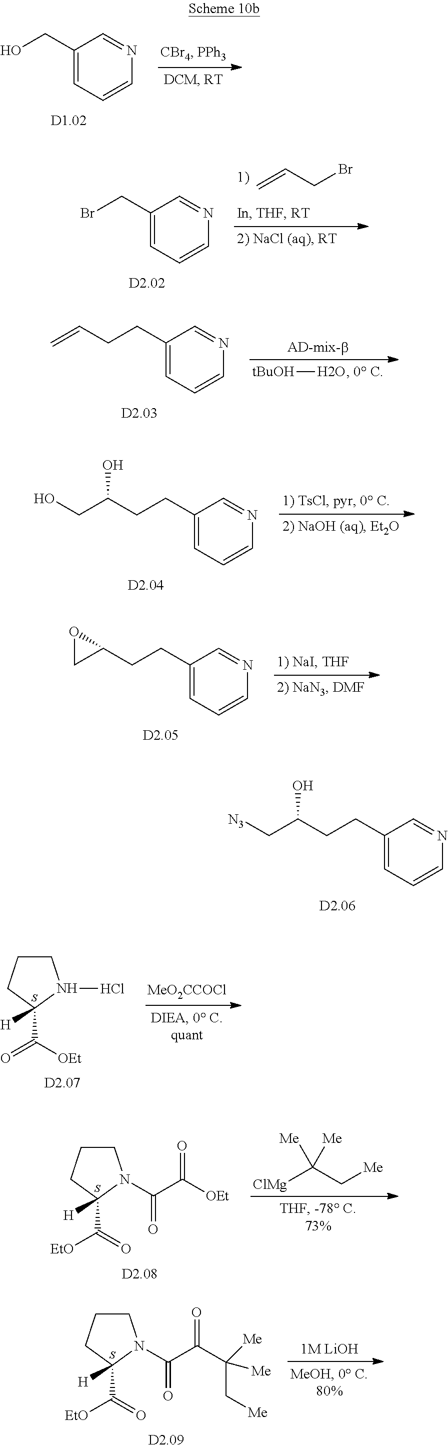

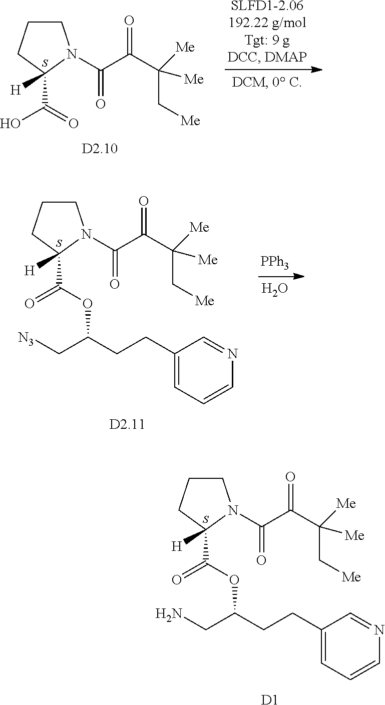

### Example 10c

### Synthesis of D2

### Compound D2 is prepared according to the method shown in Scheme 10c

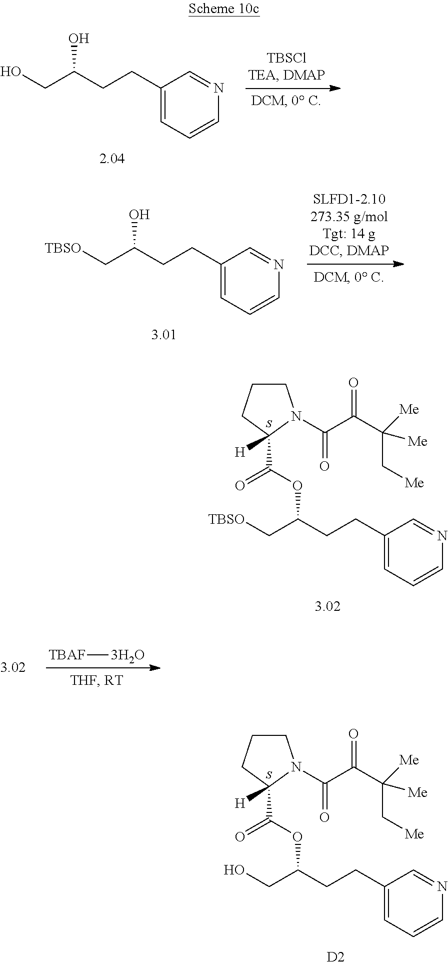

### Example 10d

### Synthesis of E1

Compound E1 is prepared according to the method shown in Scheme 10d.

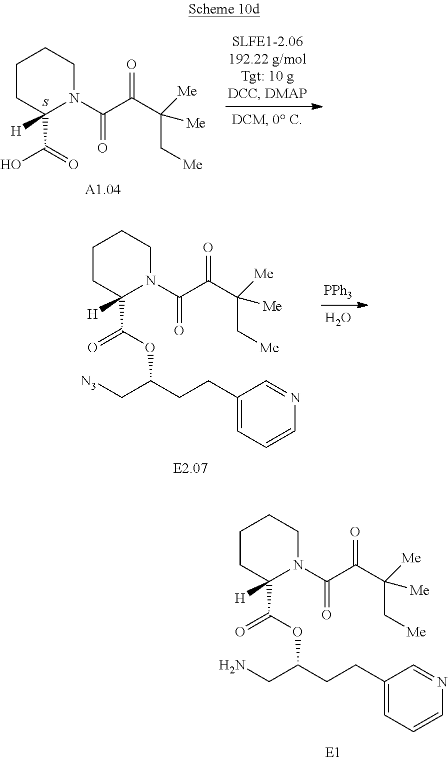

### Example 10e

### Synthesis of E2

Compound E2 is prepared according to the method shown in Scheme 10e.

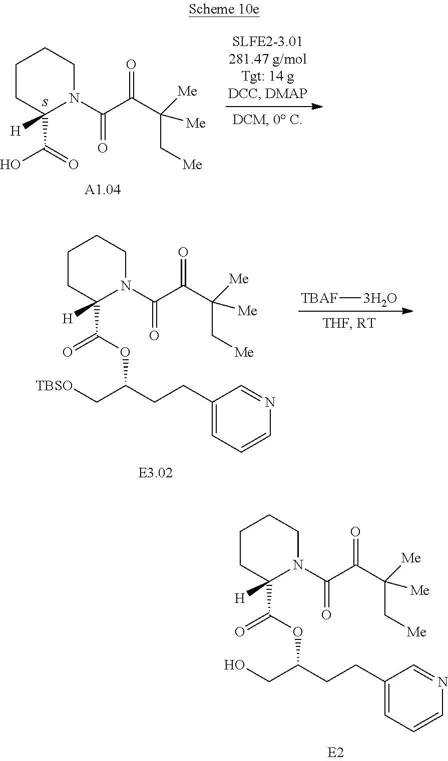

### Example 11

### Synthesis of A2.001

Compound A2.001 is prepared according to the method shown in Scheme 11.

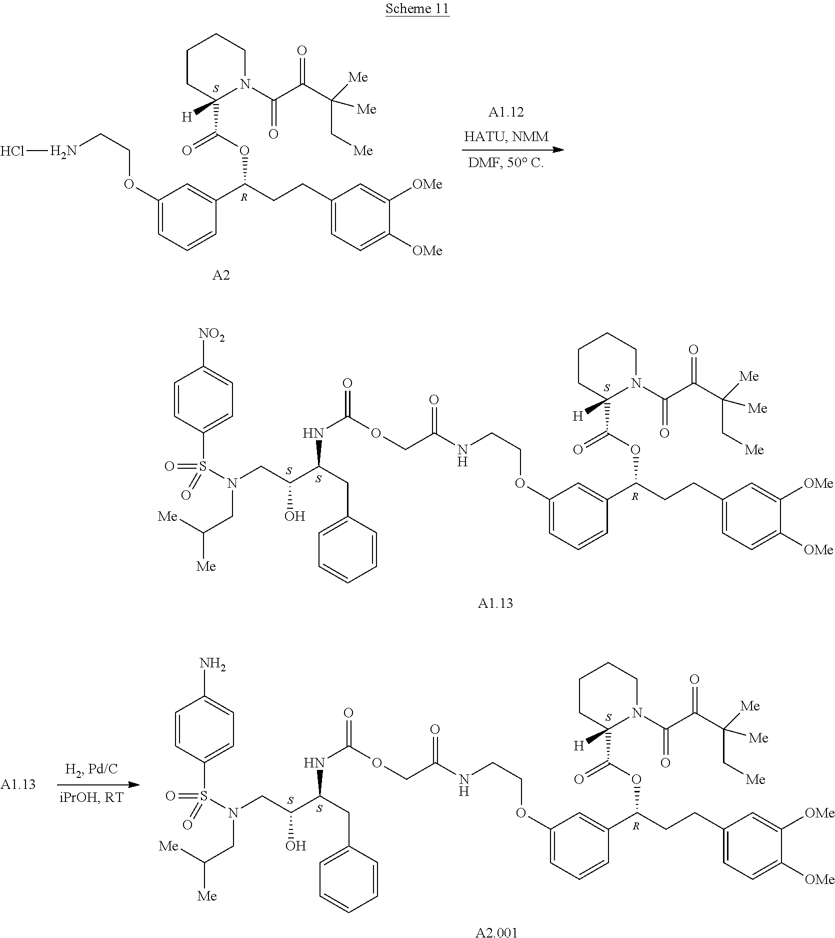

Alternative reaction conditions for the reduction reaction converting A1.13 to A2.001 utilize SnCl₂-2H₂O in EtOAc solvent at 70° C.

### Example 12

### Synthesis of A2.002

Compound A2.002 is prepared according to the method shown in Scheme 12.

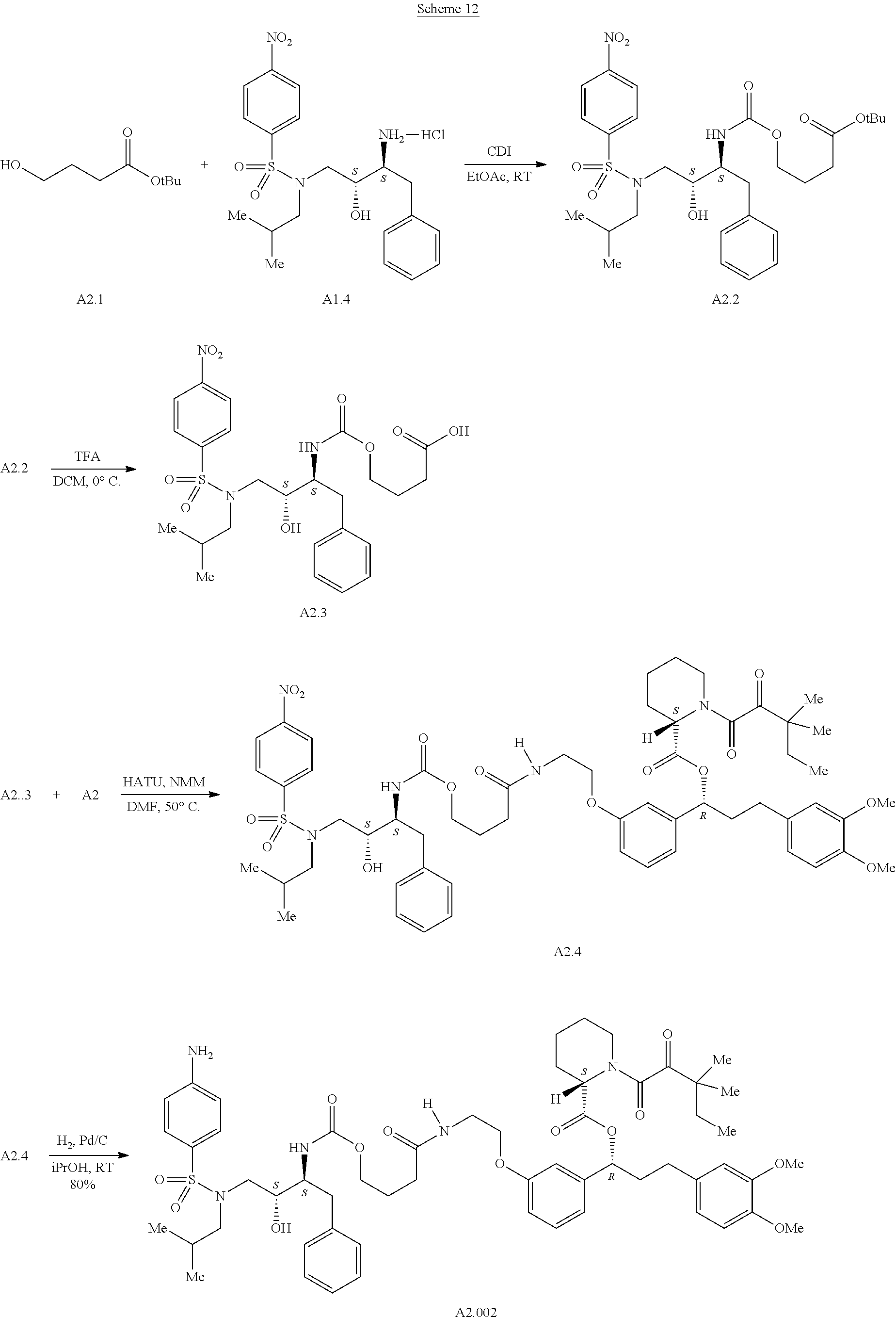

### Example 13

### Synthesis of A2.003

Compound A2.003 is prepared according to the method shown in Scheme 13.

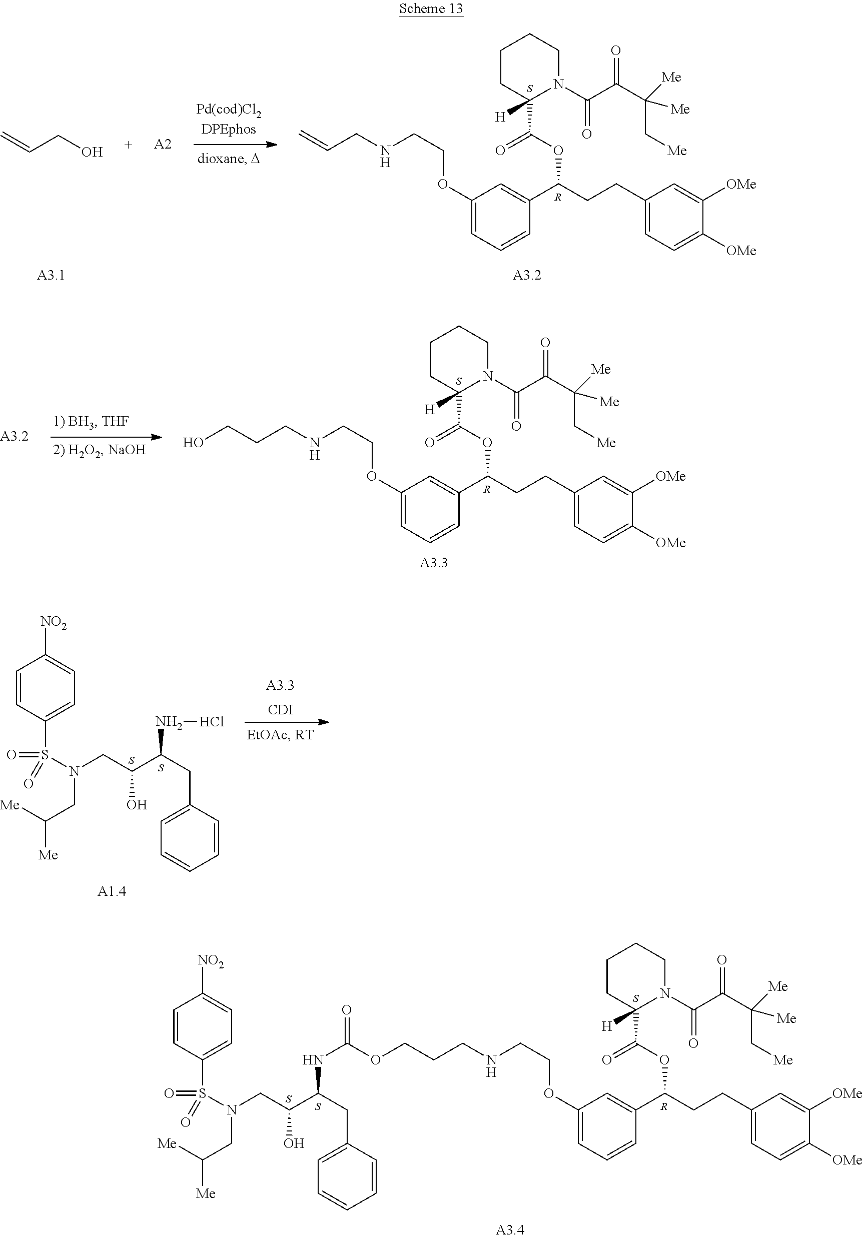

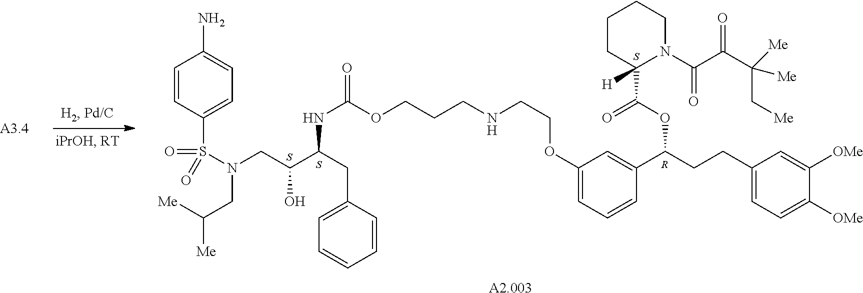

### Example 14

### Synthesis of A2.003

Compound A2.003 is prepared according to the method shown in Scheme 14.

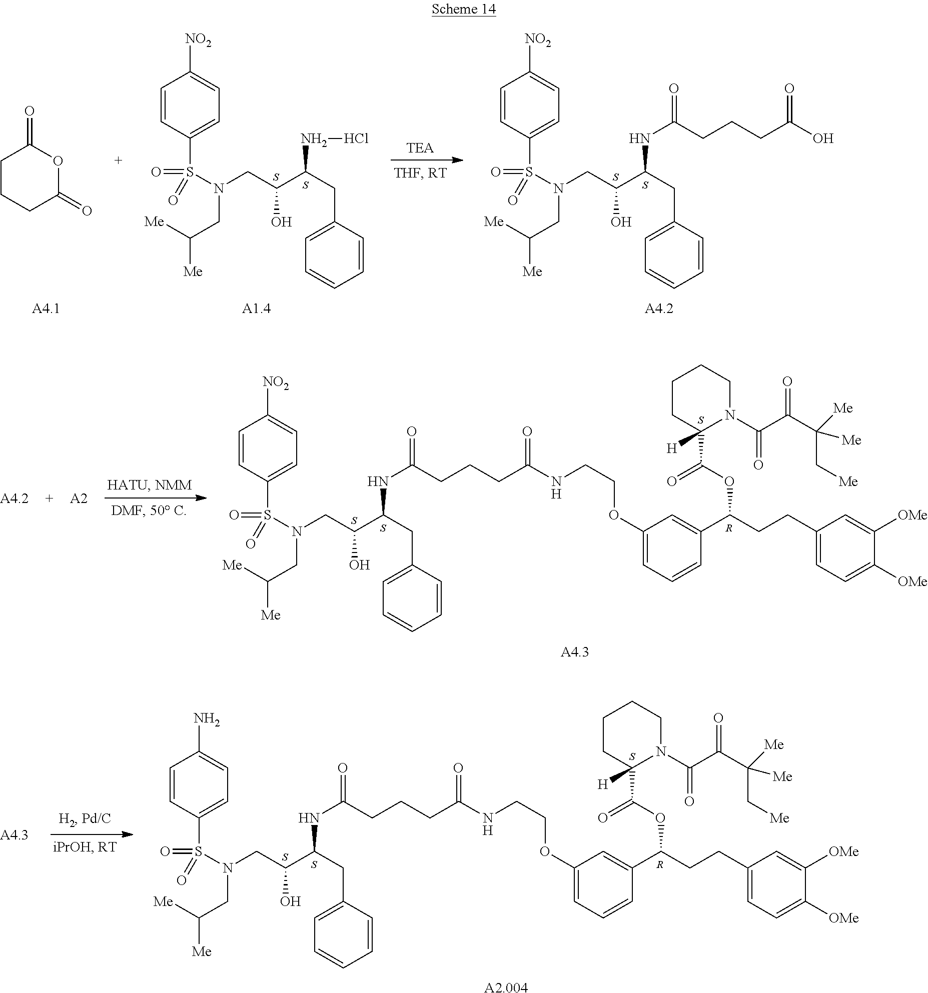

### Example 15

### Synthesis of A2.005

Compound A2.005 is prepared according to the method shown in Scheme 15.

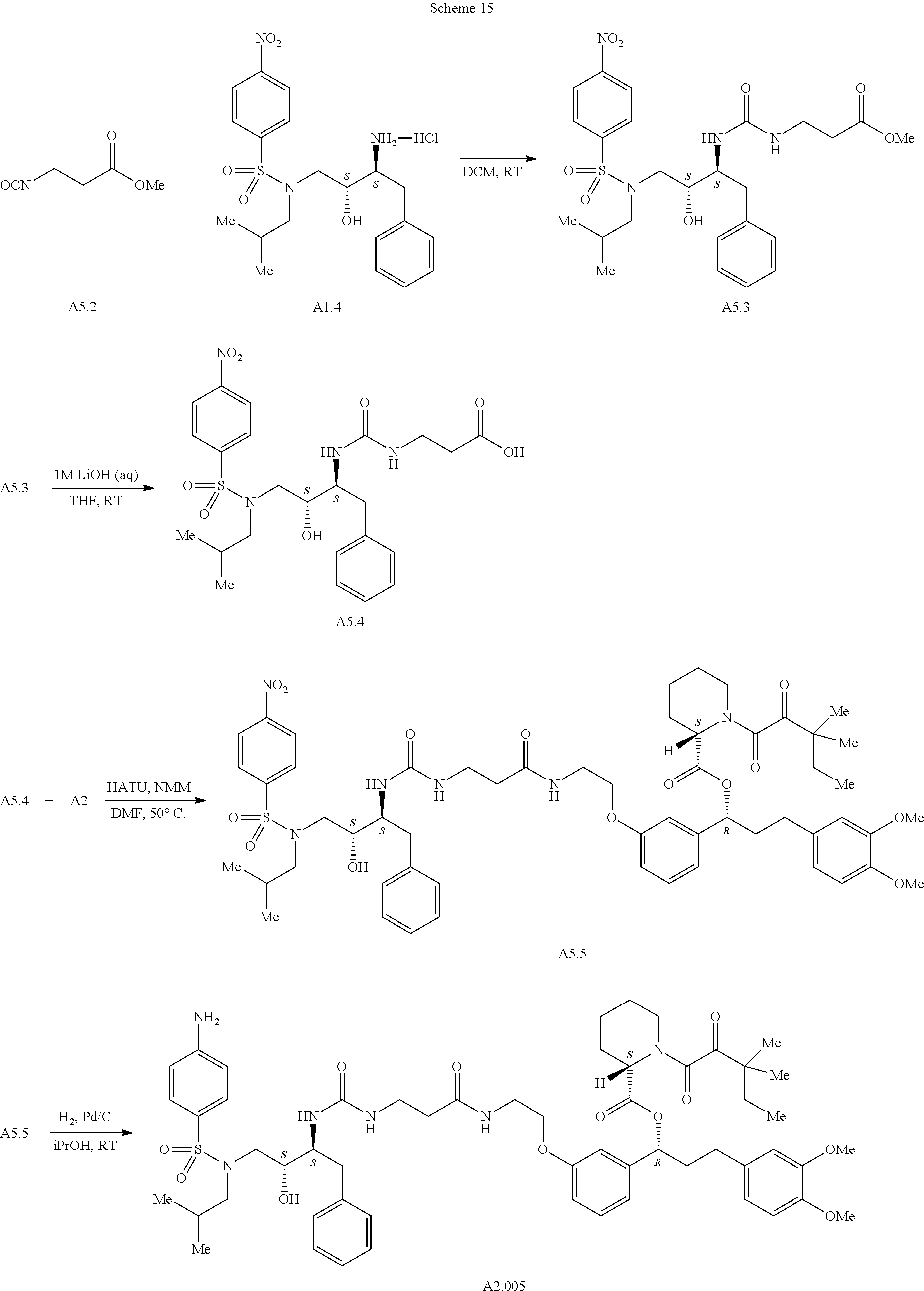

### Example 16

### Synthesis of A2.006

Compound A2.006 is prepared according to the method shown in Scheme 16.

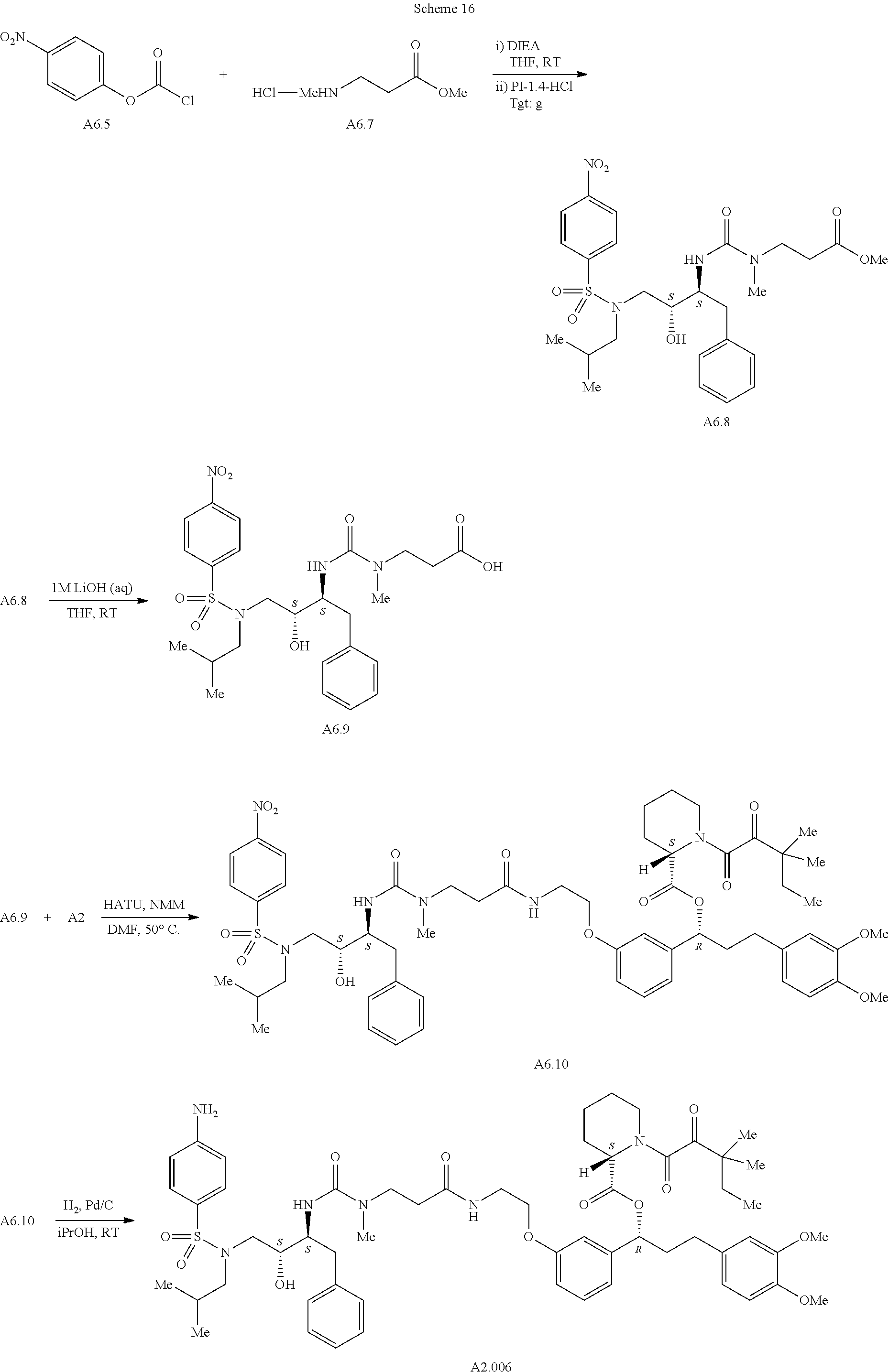

### Example 17

### Synthesis of A2.007

Compound A2.007 is prepared according to the method shown in Scheme 17.

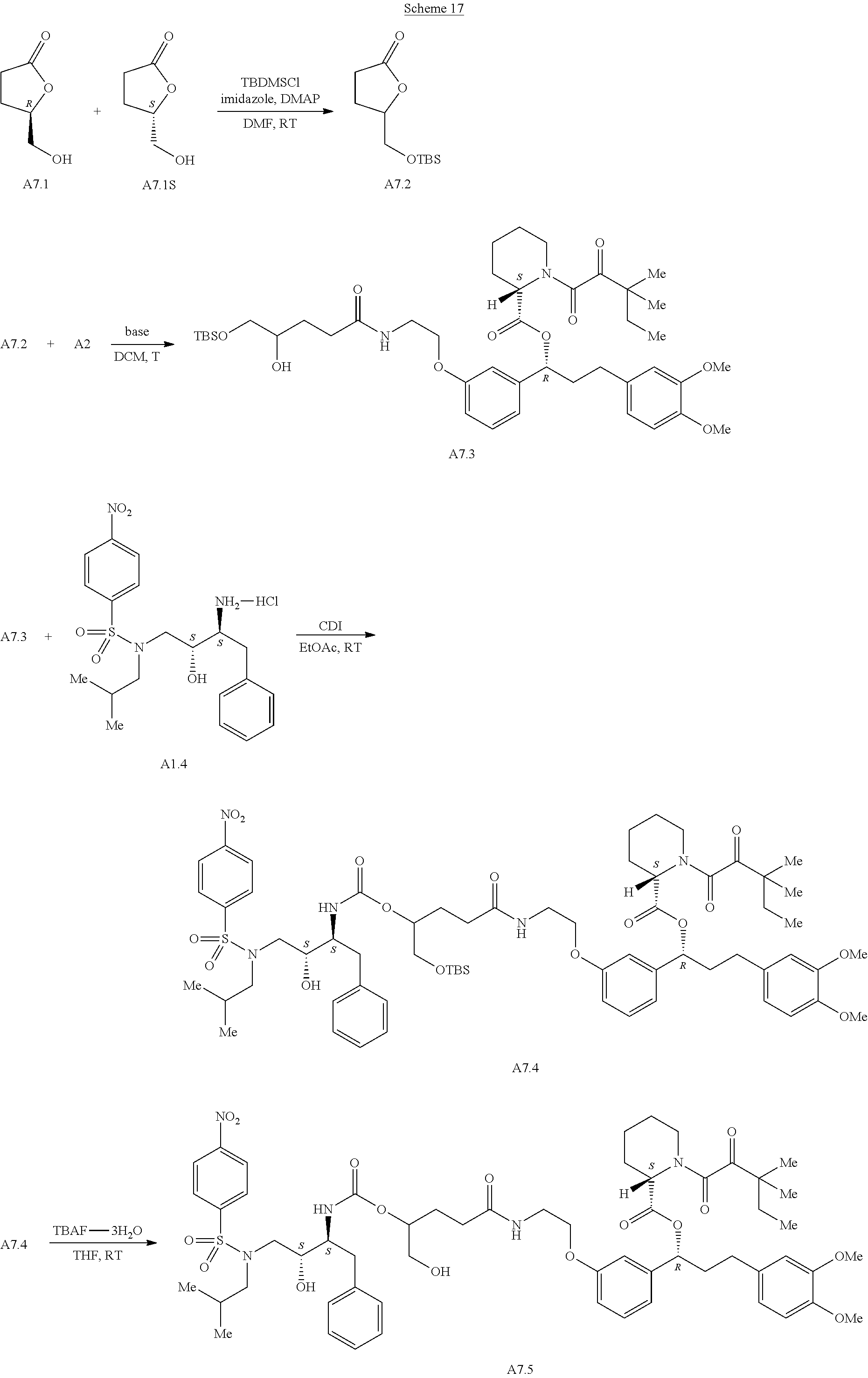

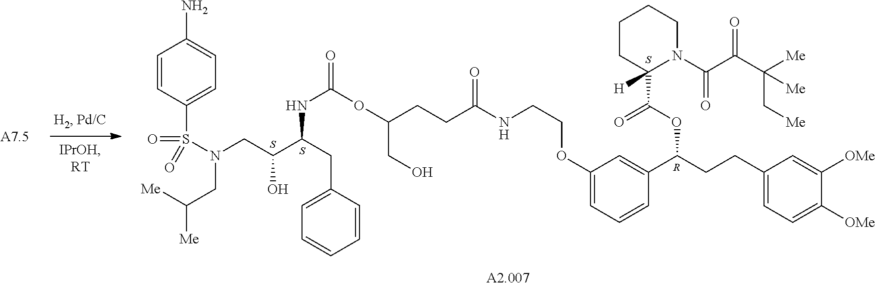

### Example 18

### Synthesis of A2.008

Compound A2.008 is prepared according to the method shown in Scheme 18.

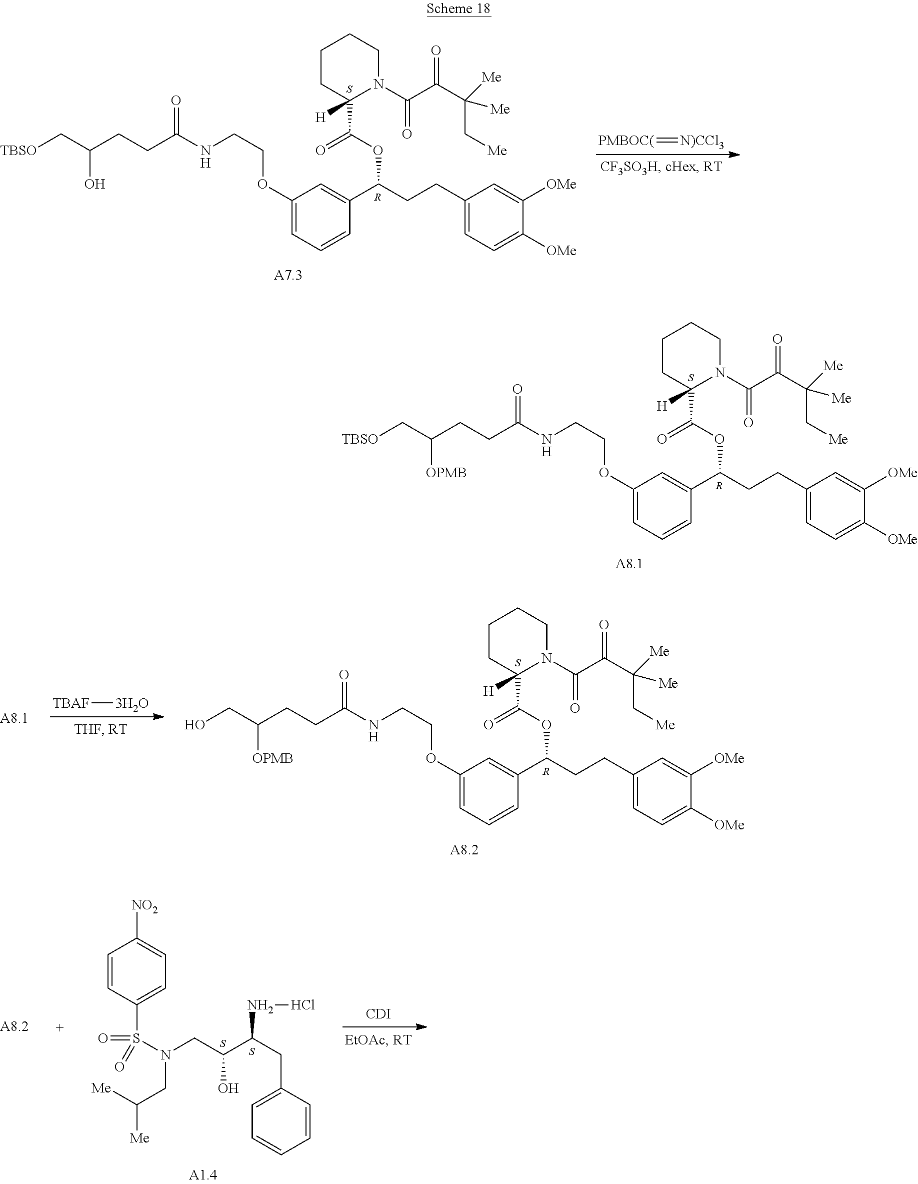

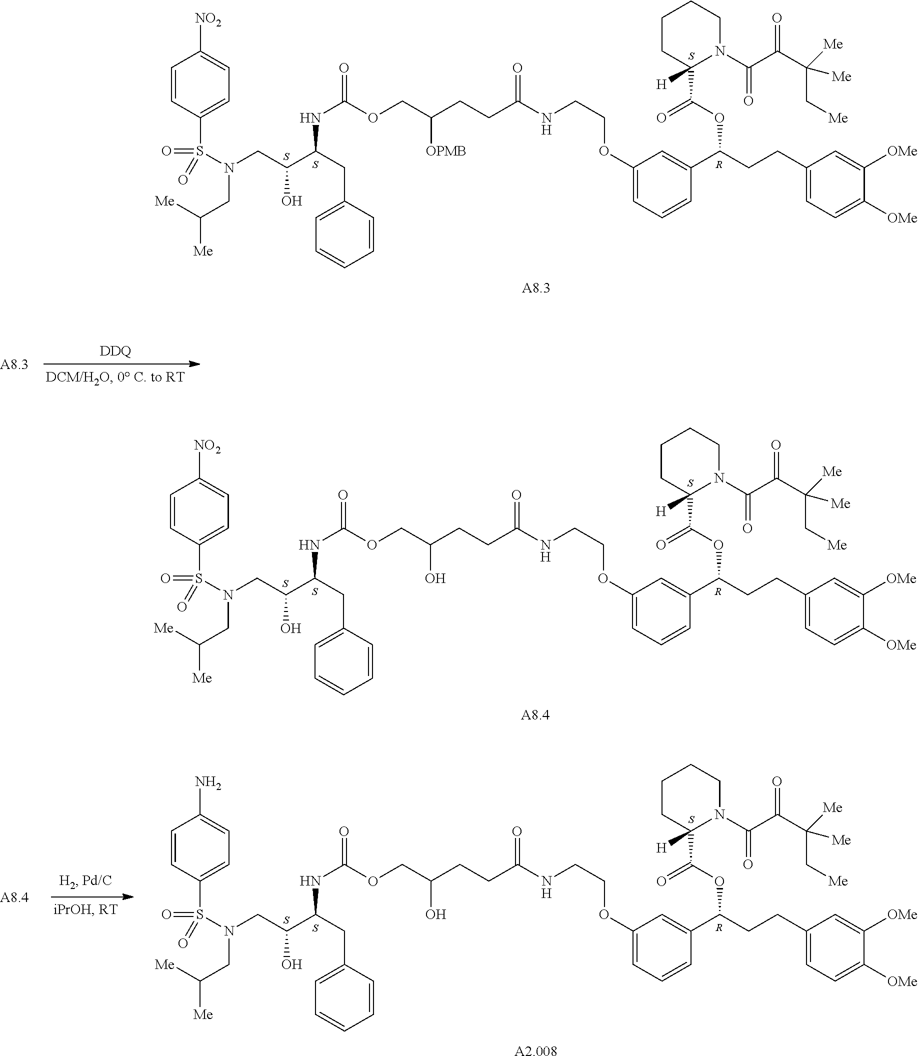

### Example 19

### Synthesis of A2.009

Compound A2.009 is prepared according to the method shown in Scheme 19.

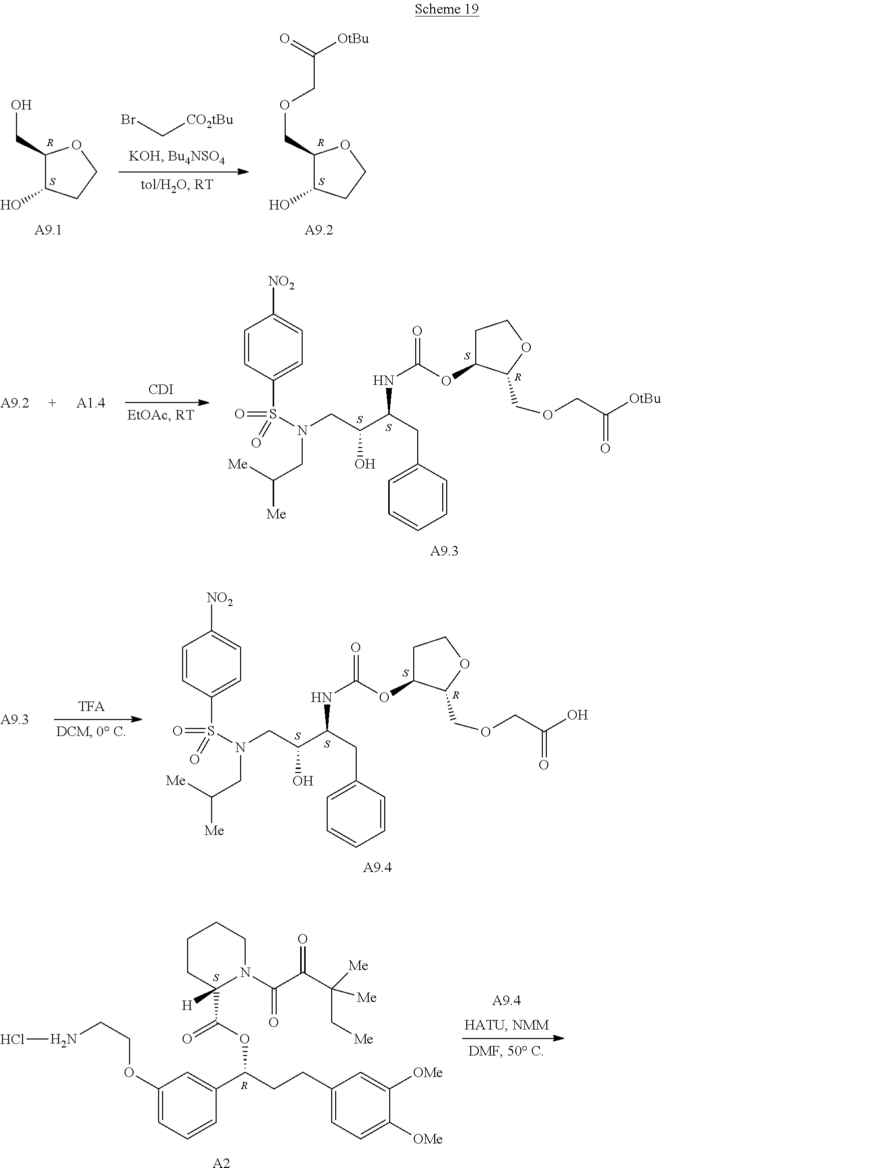

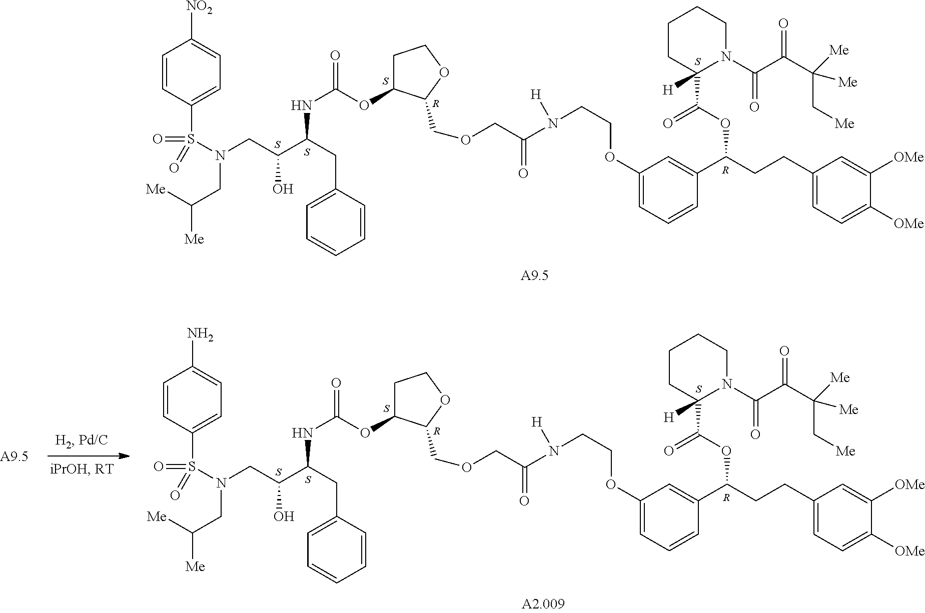

### Example 20

### Synthesis of A3.001

Compound A3.001 is prepared according to the method shown in Scheme 20.

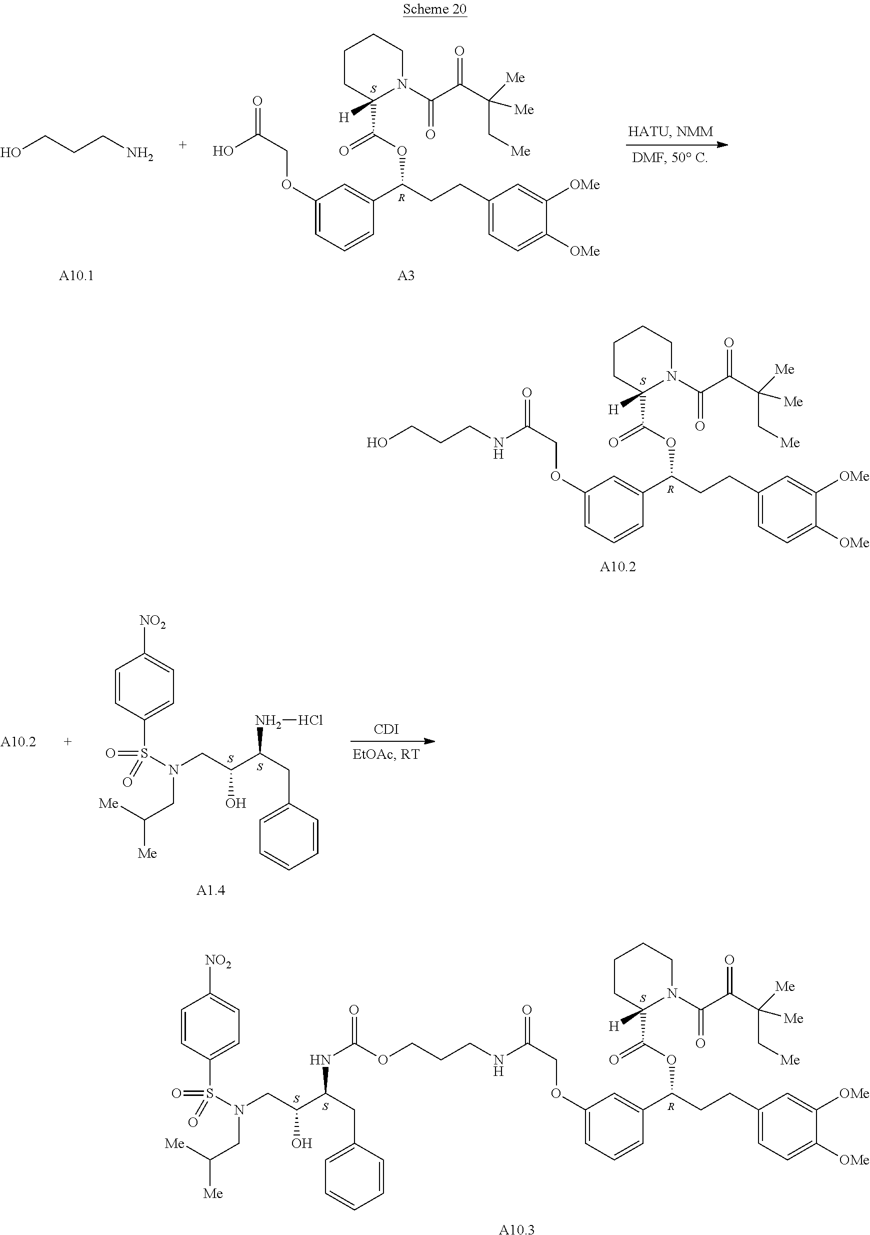

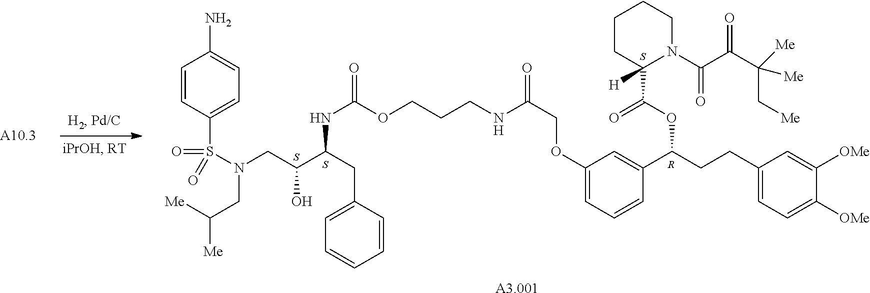

### Example 21

### Synthesis of A3.002

Compound A3.002 is prepared according to the method shown in Scheme 21.

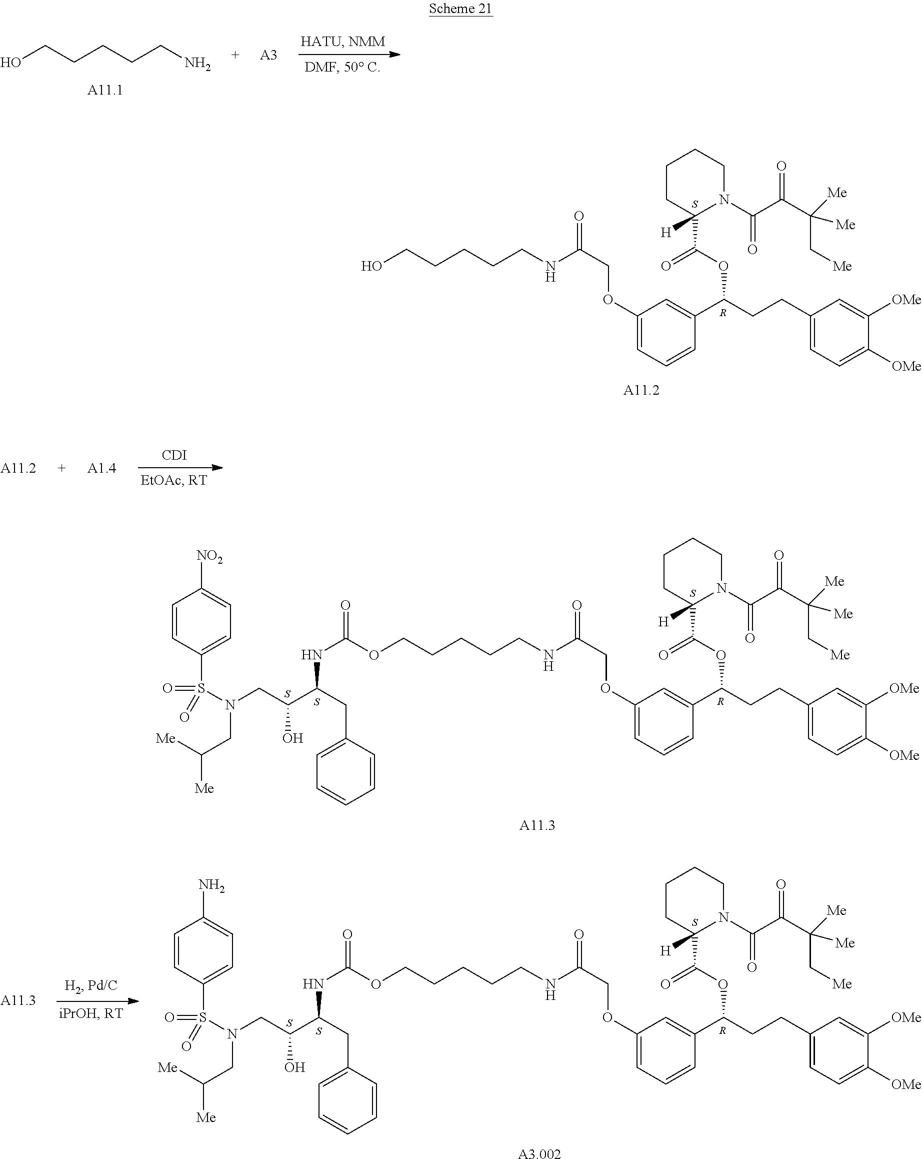

### Example 22

### Synthesis of A3.003

Compound A3.003 is prepared according to the method shown in Scheme 22.

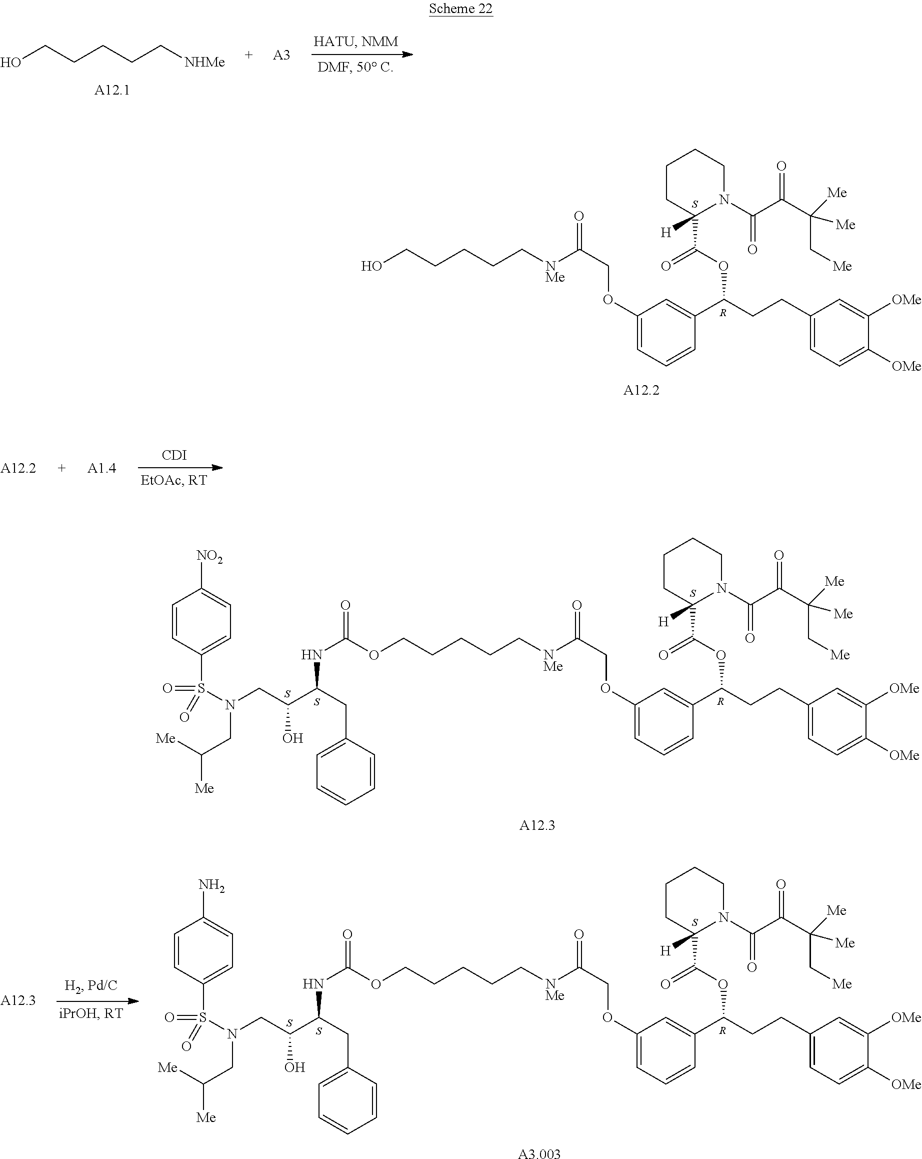

### Example 23

### Synthesis of A2.018

Compound A2.018 is prepared according to the method shown in Scheme 23.

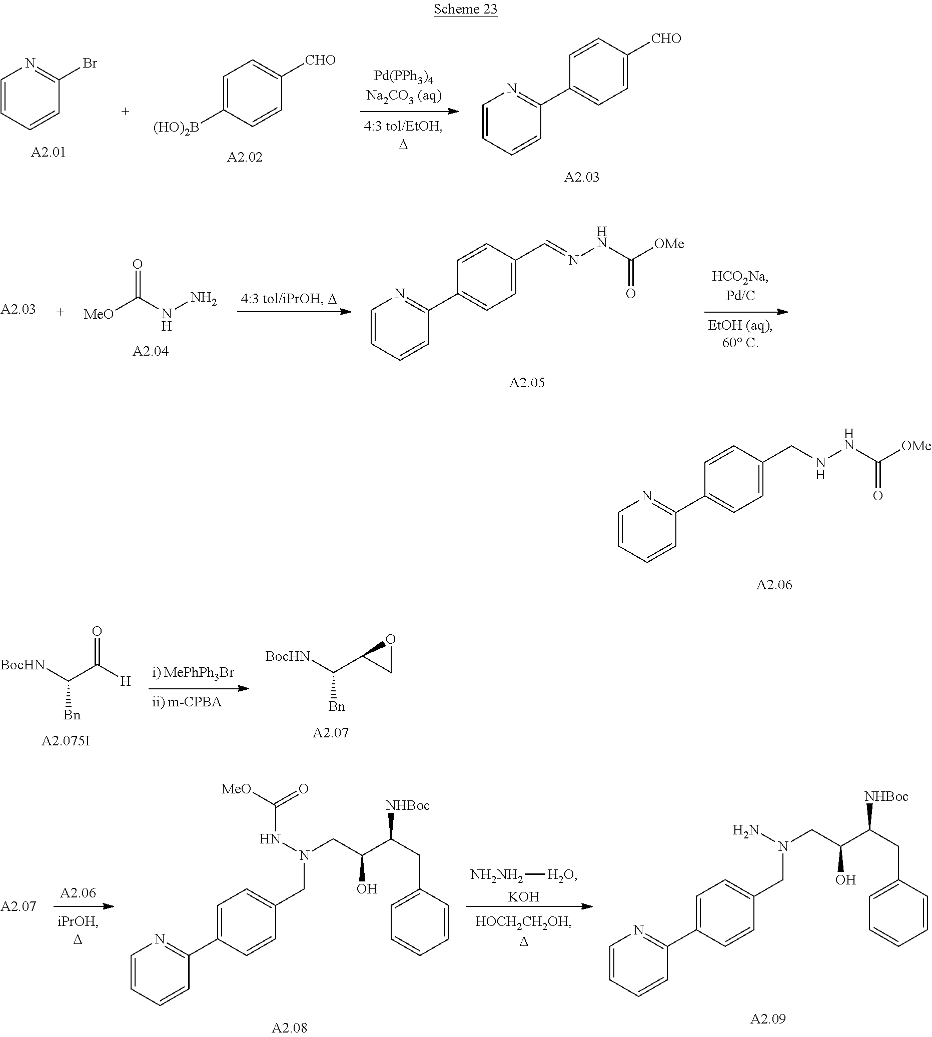

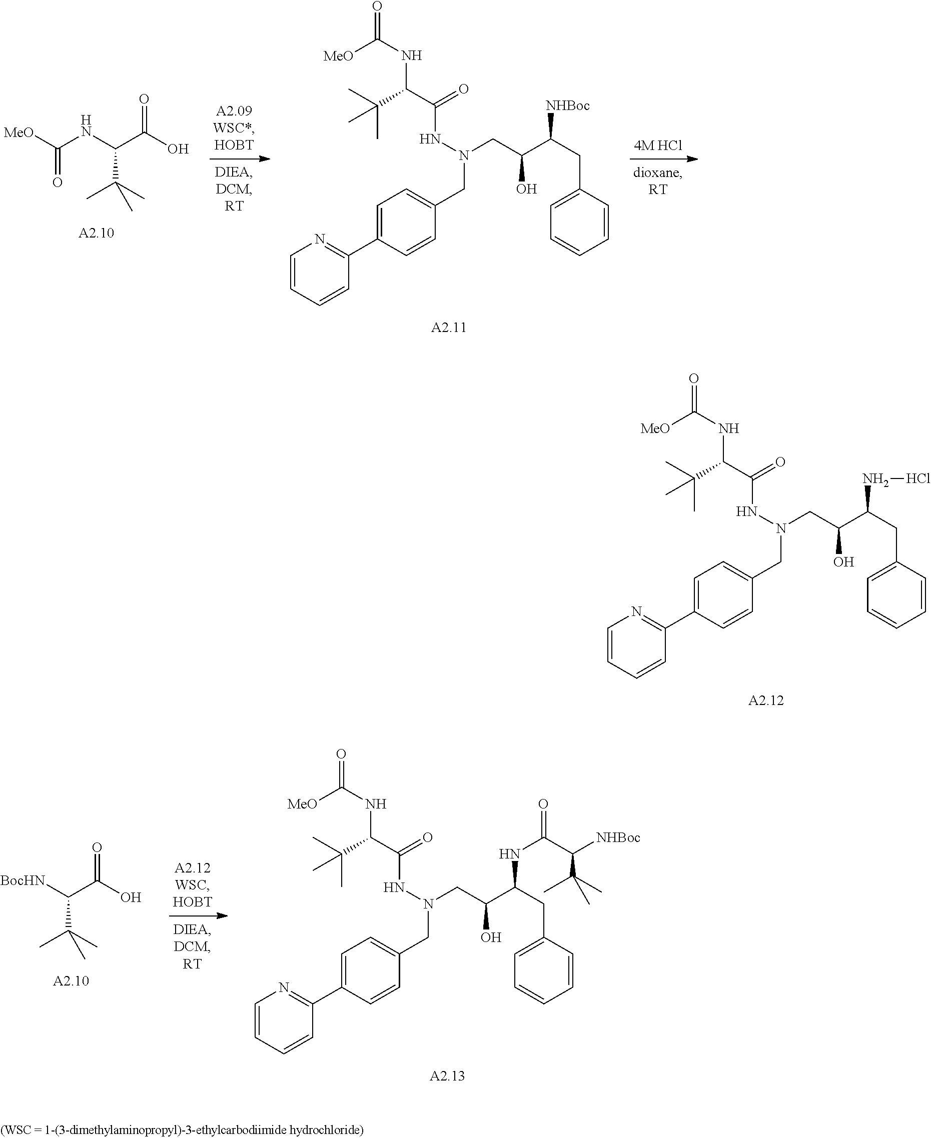

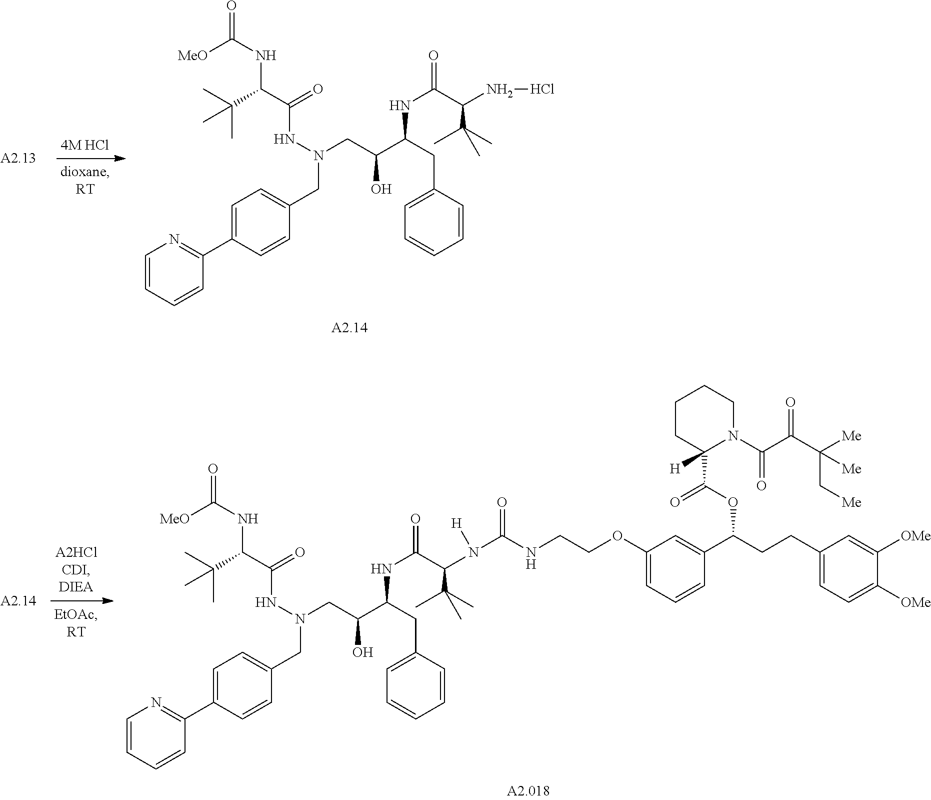

### Example 24

### Synthesis of A3.010

Compound A3.010 is prepared according to the method shown in Scheme 24.

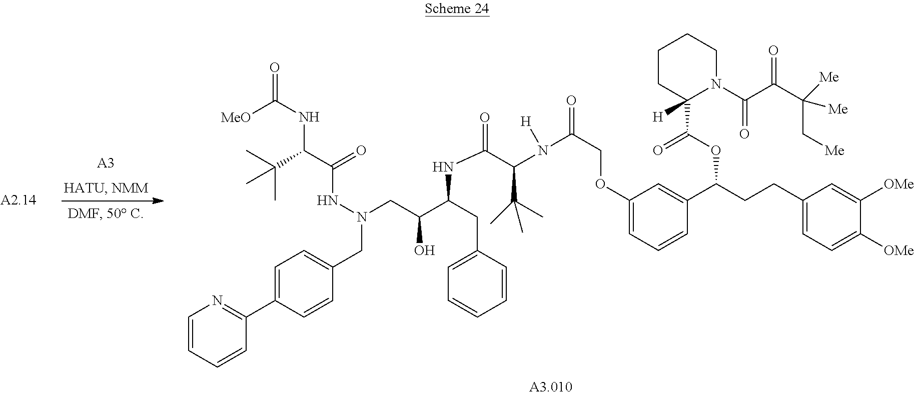

### Example 25

### Synthesis of A2.019

Compound A2.019 is prepared according to the method shown in Scheme 25.

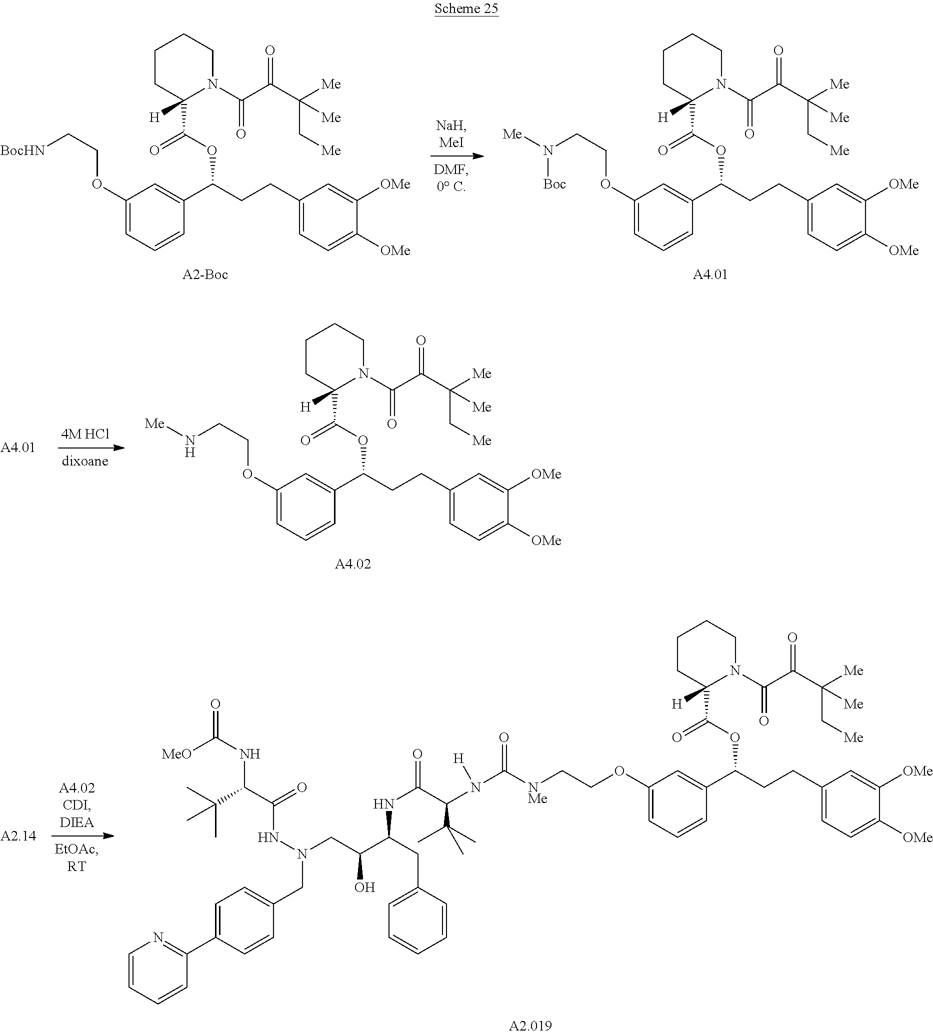

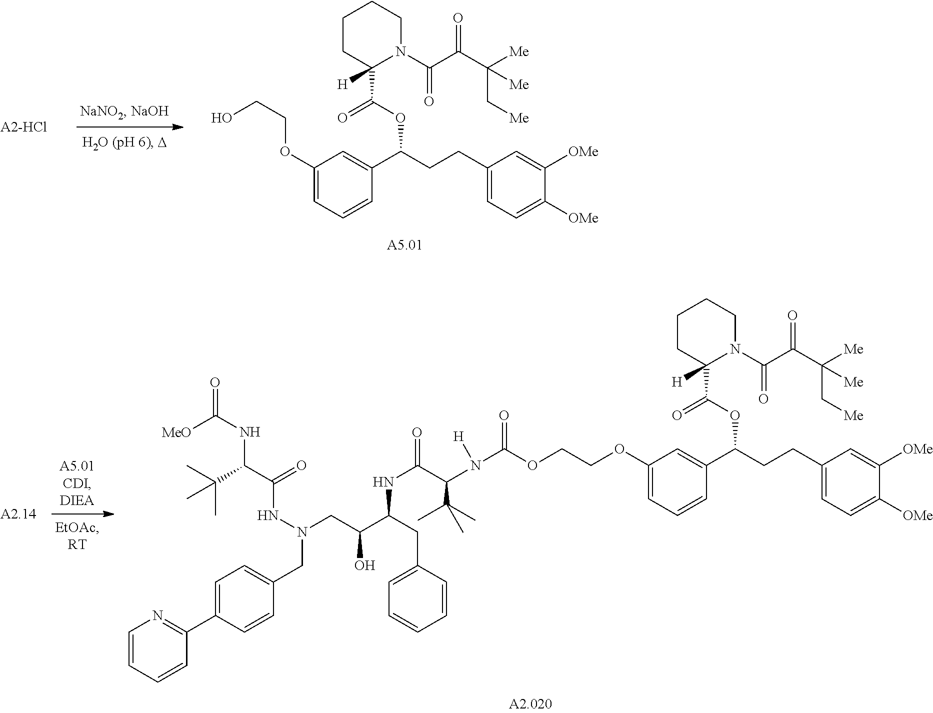

### Example 26

### Cell Free and Cell Infectivity Assays

Procedure—cell free assay. The cell free assays were conducted according to the test kit protocol provided with the SensoLyte™ 490 HIV-1 Protease Assay Kit, available from AnaSpec (San Jose, Calif.).

Procedure—cell infectivity assay. The T-cell-tropic strain HIV-1LAI was used to infect CEM-T4 cells over a dose range of the protease inhibitor compounds. CEM-T4 cells were grown in RPMI 1640 medium supplemented with 10% heat-inactivated fetal bovine serum, penicillin (100 units/mL), streptomycin (100 ug/mL), and polybrene (2 ug/mL) at 37° C. with 5% CO2. The PI dose range covered a total of nine 3:1 dilutions from 5000 nM to less than 1 nM. The tittered virus was added to wells of CEM cells (pre-treated with protease inhibitor [PI] dilution series for 1 h) at a low multiplicity of infection (MOI=0.01) and incubated for 4 h at 37° C. The cells then were washed three times with PBS (GIBCO/BRL), resuspended in triplicate wells each with 1 ml of culture medium containing the same concentration of PI as the initial pre-incubation and further incubated at 37° C. in 5% CO₂. Each well of the 24-well plate contains 1×10̂5 cells upon incubation initiation. The cells were fed every two days with fresh PI at the appropriate concentration. Samples were scored for cytopathic effect on day 4, and if consistent CPE was seen throughout the various wells, the supernatants was collected for p24 assay. Otherwise, the cultures were maintained with feeding every other day until day 8, at which point the p24 were initiated.

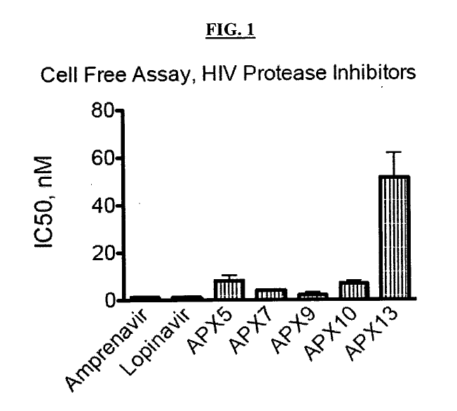

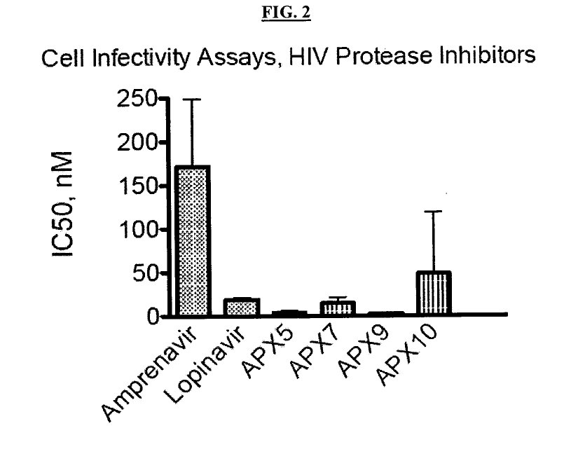

Using the above procedures, the IC₅₀ values of compounds according to the disclosure were obtained in cell free assays and cell infectivity assays. The values were compared with the IC₅₀ values for Amprenavir and Lopinavir. The results are shown in FIGS. 1 and 2, and are tabulated in Table 1. The structures of amprenavir and lopinavir are provided below.

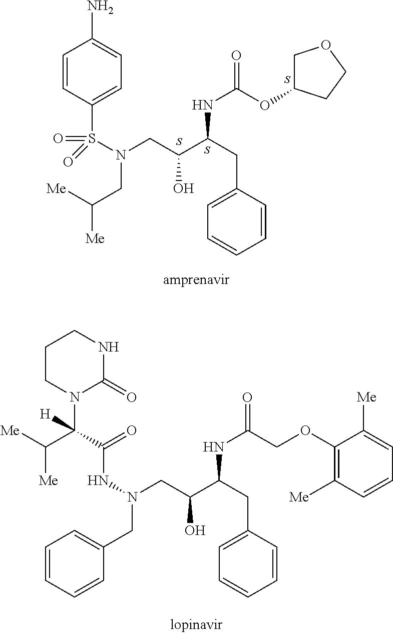

TABLE 1
Activity data

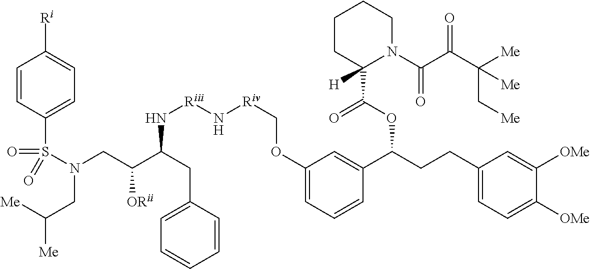

| | | | | | HIV PI | HIV PI IC₅₀ |
|---|---|---|---|---|---|---|
| | | | | | IC₅₀ (nM) | (nM) (Cell |
| | | | | | (Cell free | Infectivity |
| Compound | Rⁱ | Rⁱⁱ | Rⁱⁱⁱ | R^{iv} | assay) | Assay) |
| A5 | NH₂ | H | 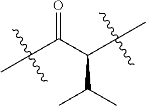 | 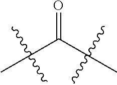 | 7.9 | 3.3 |
| A7 | NH₂ | Me | 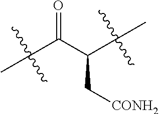 | 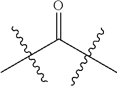 | 6.6 | 14 |
| A9 | NH₂ | Me | 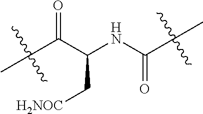 | —CH₂— | 1.8 | 2.3 |
| A10 | NH₂ | H | 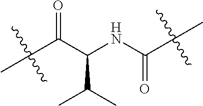 | —CH₂— | 3.9 | 48.1 |

| | | |
|---|---|---|
| Amprenavir | 1.0 | 171 |
| Lopinavir | 0.9 | 18.9 |

Based on the tabulated data, it can be seen that modifications to the amprenavir structure resulted in changes in potency. In particular, removal of the tetrahydrofuran moiety from amprenavir resulted in increased potency.

Furthermore, compounds A5, A7, and A9 exhibited little or no loss in potency between cell free assay experiments and cell infectivity assay experiments. In contrast, amprenavir and lopinavir exhibited significant loss of potency between cell free assays and cell infectivity assays.

### Example 27

### Assays and Additional Compounds

Compounds A1, A2, A3, A4, A6, and A14 were prepared, and IC₅₀ values for Cell Free assays were obtained according to the procedure outlined above. The results are provided in Table 2.

### Table 2. Activity Data

TABLE 2
Activity Data

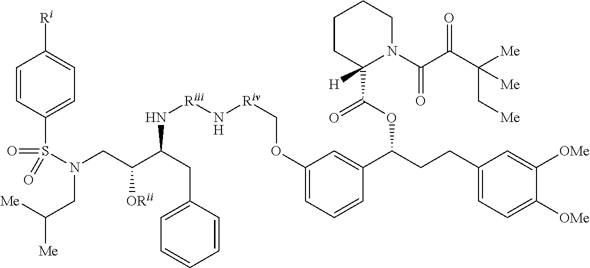

| | | | | | HIV PI IC₅₀ |
|---|---|---|---|---|---|
| | | | | | (nM) (Cell- |
| Compound | Rⁱ | Rⁱⁱ | Rⁱⁱⁱ | R^{iv} | Free Assay) |
| A1 | NO₂ | H | 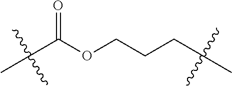 | 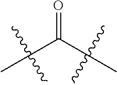 | 24 |
| A2 | NH₂ | H | 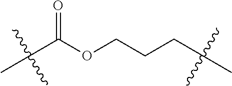 | 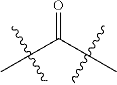 | 7.3 |
| A3 | NO₂ | H | 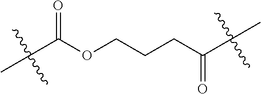 | —CH₂— | 11.5 |
| A4 | OMe | H | 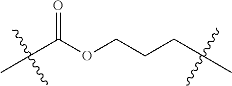 | 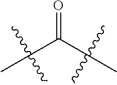 | 8.4 |
| A6 | NH₂ | H | 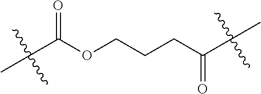 | —CH₂— | 5.6 |
| A13 | NH₂ | H | 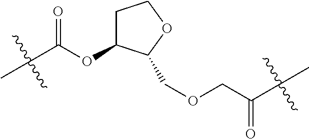 | —CH₂— | 51 |
| A14 | NH₂ | H | 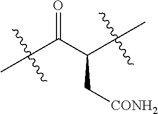 | 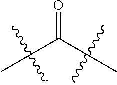 | 3.5 |

In addition to the compounds prepared and described in Table 2, compound A8 (structure shown below) was prepared and found to have an IC₅₀ value of 339 nM. As the data shows, the prepared compounds exhibited activity against HIV protease. Simple modification to provide a structurally similar analog of amprenavir resulted in a compound with less potency (i.e., A13) compared to analogs having less structural similarity.

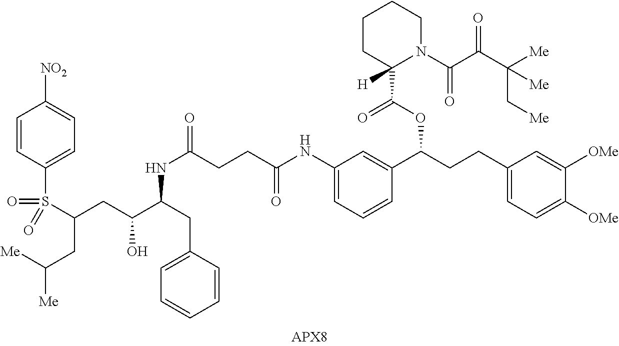

1. A compound having the structure of formula (A) 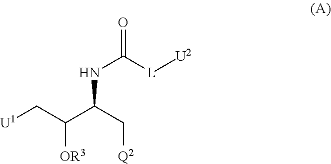
wherein:
Q² is selected from alkyl and aryl;
R³ is selected from H, hydrocarbyl, functional groups, hydroxyl-protecting groups, and inorganic acid groups;
U¹ is selected from hydrocarbyl and functional groups;
L is a linking moiety selected from hydrocarbylene and functional groups;
U² is a group selected from Units A, and B: 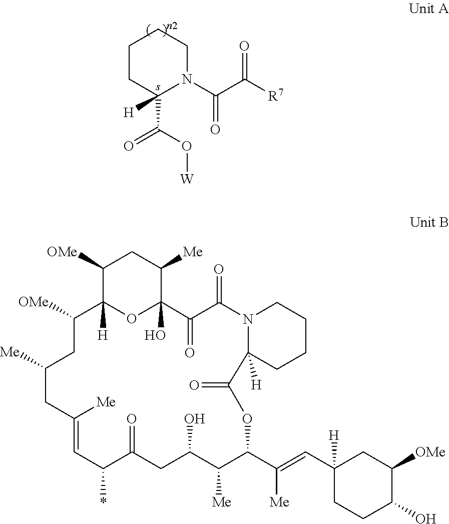
wherein
W is a linker moiety connecting Unit A with L and is selected from a bond, alkylene, arylene, and 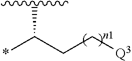
n1 is an integer selected from 1 and 2;
n2 is an integer from 0 to 2;
R⁷ is selected from H, hydrocarbyl, and functional groups;
Q³ is selected from aryl and alkyl; and
the stars represent the point of connection to L and the wavy line represents the point of connection to Unit A,
as well as pharmaceutically acceptable salts, prodrugs, and metabolites thereof.

2. The compound of claim 1, wherein U¹ is selected from 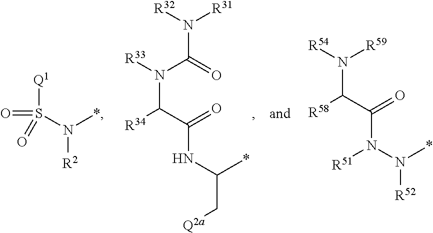
wherein:
the stars represent connection points to the remainder of formula (I);
Q¹ is selected from an aromatic group, an alicyclic group, and an amine group;
Q^{2a} is selected from substituted or unsubstituted alkyl, substituted or unsubstituted heteroatom-containing alkyl, substituted or unsubstituted aryl, and substituted or unsubstituted heteroaryl;
R² is selected from H, hydrocarbyl, and functional groups;
R³¹, R³², R³³, and R³⁴ are independently selected from H and hydrocarbyl, and wherein any two of R³¹, R³², R³³, and R³⁴ may be taken together to form a ring;
R⁵¹ is selected from H and alkyl;
R⁵² is selected from alkyl, aryl, aralkyl, and alkaryl;
R⁵⁴ is selected from —C(═O)-A-R⁵⁰ and Ar⁵, wherein A is selected from a bond, —O—, —NR⁵⁵—, R⁵⁰ is alkyl, and Ar⁵ is aryl;
R⁵⁵ is H or lower alkyl;
R⁵⁸ is alkyl, aryl, aralkyl, or alkaryl;
R⁵⁹ is H or alkyl, and wherein any two of R⁵¹, R⁵², R⁵⁴, R⁵⁸, and R⁵⁹ may be taken together to form a ring.

3. The compound of claim 2, wherein the compound has the structure of formula (Ia) 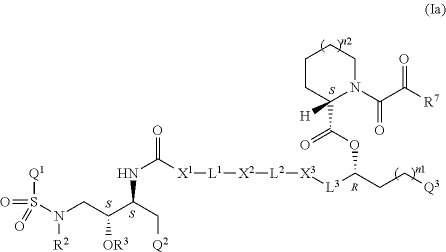
wherein:
Q³ is selected from aromatic groups and alkyl groups;
n1 is an integer selected from 1 and 2;
n2 is an integer selected from 0, 1, and 2;
R⁷ is selected from H, hydrocarbyl, and functional groups;
X¹ is selected from a bond, —O— and —NR¹⁰—, wherein R¹⁰ is selected from H and lower alkyl;
L¹ is selected from alkylene, arylene, alkarylene, and aralkylene;
X² is selected from a bond and —NR¹¹—, wherein R¹¹ is selected from H and lower alkyl;
L² is alkylene;
X³ is selected from —O—, and —NR¹²—, wherein R¹² is selected from H and lower alkyl, and
L³ is selected from an arylene group and an alkylene group.

4-8. (canceled)

9. The compound of claim 3, wherein L³ is selected from methylene, arylene, biaryl, heteroarylene, and heterobiarylene, any of which may be substituted or unsubstituted.

10. (canceled)

11. The compound of claim 3, wherein L¹ is selected from substituted or unsubstituted alkylene, substituted or unsubstituted heteroatom-containing alkylene, an amino acid linking moiety, substituted or unsubstituted cycloalkylene, substituted or unsubstituted heteroatom-containing cycloalkylene, substituted or unsubstituted arylene, substituted or unsubstituted heteroarylene, substituted or unsubstituted aralkylene, substituted or unsubstituted heteroatom-containing aralkylene, substituted or unsubstituted alkarylene, and substituted or unsubstituted heteroatom-containing alkarylene,

12. The compound of claim 11, wherein L¹ has the structure of formula (L1a) 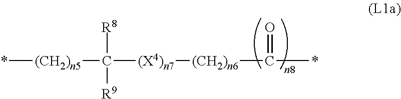
wherein:
the stars represent connection points to the remainder of the compound;
X⁴ is selected from —O— and —NR¹⁵—;
R⁸, and R⁹ are independently selected from H, substituted or unsubstituted lower alkyl, substituted or unsubstituted heteroatom-containing lower alkyl,
R¹⁵ is H or lower alkyl;
n7 and n8 are independently 0 or 1; and
n5 and n6 are independently selected from an integer in the range of 0-12.

13. The compound of claim 11, wherein L¹ has the structure of —Ar¹—X⁵—, wherein Ar¹ is a substituted or unsubstituted 5- or 6-membered aromatic ring optionally containing one or more heteroatoms, and X⁵ is selected from a bond, —C(═O)—, —CH₂—, and —S(═O)₂—.

14. The compound of claim 11, wherein L¹ has the structure of formula (L1i) 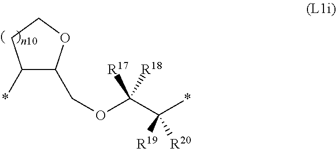
wherein:
the stars represent connection points to the remainder of the compound;
n10 is an integer selected from 1 and 2; and
R¹⁷, R¹⁸, R¹⁹, and R²⁰ are independently selected from H and lower alkyl.

15-17. (canceled)

18. The compound of claim 2, wherein the compound has the structure of formula (IIa) 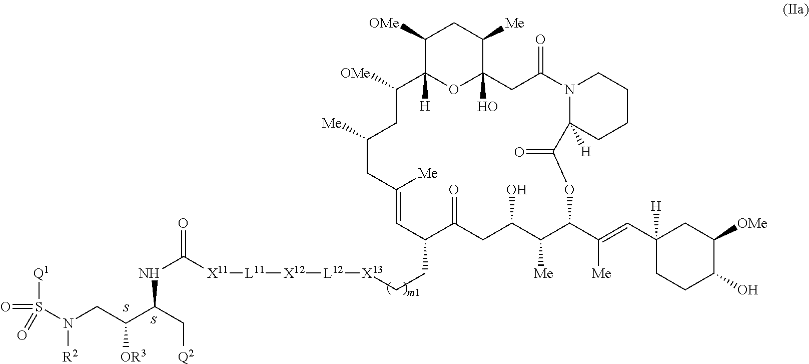
wherein:
m1 is an integer selected from 1 and 2;
X¹¹ is selected from a bond, —O— and —NR¹⁰—, wherein R¹⁰ is selected from H and lower alkyl;
L¹¹ is selected from alkylene, arylene, alkarylene, and aralkylene;
X¹² is selected from a bond and —NR¹¹—, wherein R¹¹ is selected from H and lower alkyl;
L¹² is selected from alkylene and alkenylene; and
X¹³ is selected from a bond and —O—.

19-20. (canceled)

21. The compound of claim 2, wherein the compound has the structure of formula (IIIa) 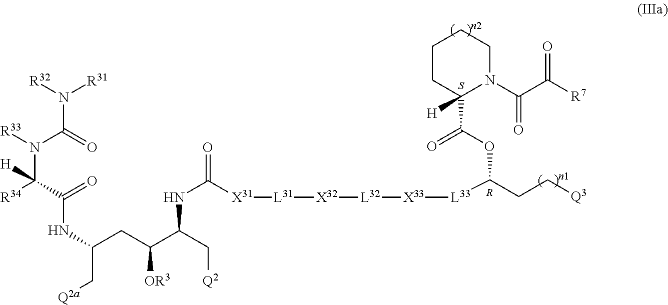
wherein, in formula (IIIa):
X³¹ is a linker selected from a bond and a hydrocarbylene group;
X³² and X³³ are independently selected from a linker selected from a bond, a hydrocarbylene group, and a functional linker group; and
L³¹, L³², and L³³ are independently selected from a bond, a hydrocarbylene group, and a functional linker group.

22. The compound of claim 1, wherein the compound has the structure of formula (IVa) 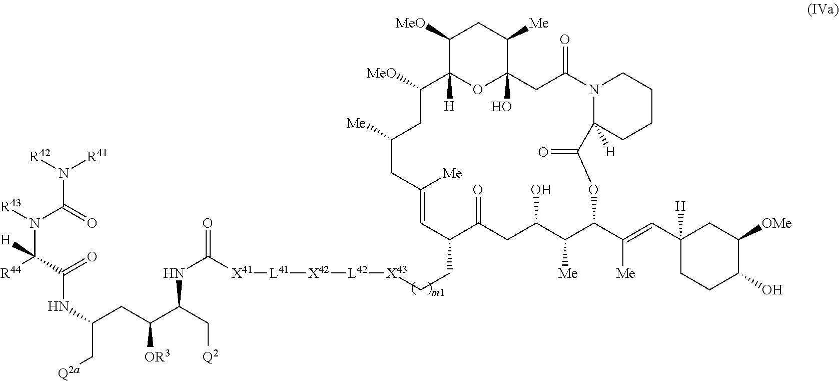
wherein, in formula (IVa):
R⁴¹, R⁴², R⁴³, and R⁴⁴ are independently selected from H and hydrocarbyl, and wherein any two of R⁴¹, R⁴², R⁴³, and R⁴⁴ may be taken together to form a ring;
m1 is an integer selected from 1 and 2;
X⁴¹ is a linker selected from a bond and a hydrocarbylene group;
X⁴² and X⁴³ are independently selected from a linker selected from a bond, a hydrocarbylene group, and a functional linker group; and
L⁴¹, and L⁴² are independently selected from a bond, a hydrocarbylene group, and a functional linker group.

23. The compound of claim 2, wherein the compound has the structure of formula (Va) 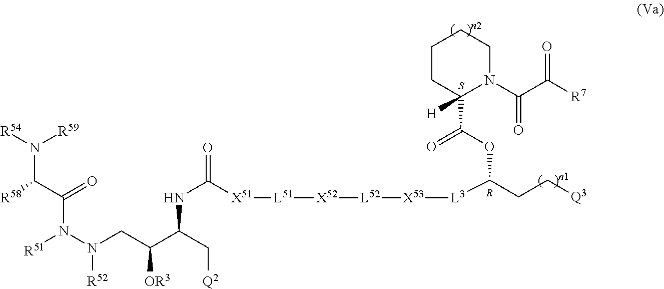
wherein, in formula (V),
L³ is selected from an arylene group and an alkylene group
X⁵¹ is selected from a bond, —O—, and —NR⁵⁶—;
L⁵¹ is alkylene;
X⁵² is selected from a bond, —O—, and —NR⁵⁶—;
L⁵² is alkylene;
X⁵³ is selected from —O— and —NR⁵⁶—; and
each R⁵⁶ is independently selected from H and alkyl.

24. The compound of claim 2, wherein the compound has the structure of formula (VIa) 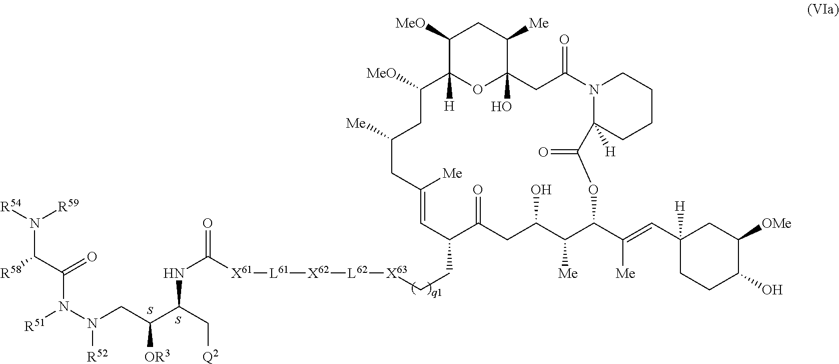
wherein, in formula (VI):
q1 is 1 or 2;
X⁶¹ is selected from a bond, —O—, and —NR⁶⁶—;
L⁶¹ is alkylene;
X⁶² is selected from a bond, —O—, and —NR⁶⁶—;
L⁶² is alkylene;
X⁶³ is selected from —O— and —NR⁶⁶—.

25. A compound having the structure of formula (B) 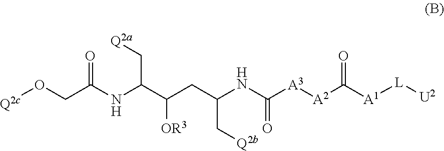
wherein
A¹ and A² are independently selected from nitrogen-containing linking moieties;
A³ is a hydrocarbylene linker;
Q^{2a}, Q^{2b}, and Q^{2c} independently selected from alkyl and aryl;
R³ is selected from H, hydrocarbyl, functional groups, hydroxyl-protecting groups, and inorganic acid groups;
L is a linking moiety selected from hydrocarbylene and functional groups;
U² is a group selected from Units A, and B: 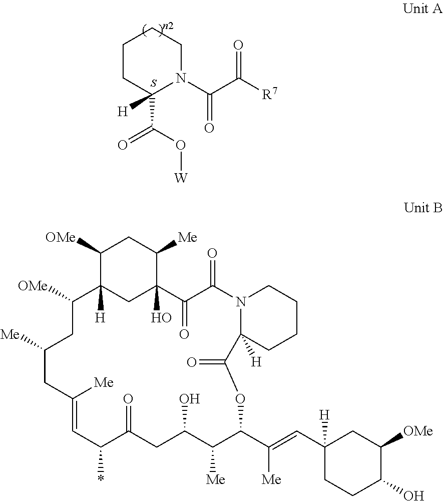
wherein
W is a linker moiety connecting Unit A with L and is selected from a bond, alkylene, arylene, and 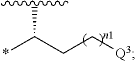
n1 is an integer selected from 1 and 2;
n2 is an integer from 0 to 2;
R⁷ is selected from H, hydrocarbyl, and functional groups;
Q³ is selected from aryl and alkyl; and
the stars represent the point of connection to L and the wavy line represents the point of connection to Unit A,
as well as pharmaceutically acceptable salts, prodrugs, and metabolites thereof.

26. The compound of claim 25, wherein the compound has the structure of formula (IIIb) 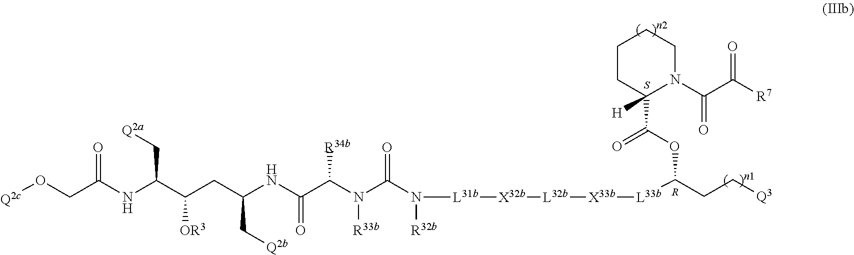
wherein, in formula (IIIb):
R^{32b}, R^{33b}, and R^{34b} are independently selected from H and hydrocarbyl, provided that any two of R^{32b}, R^{33b}, and R^{34b} may be taken together to form a ring; and
X^{32b}, X^{33b}, L^{31b}, L^{32b}, and L^{33b} are linkers independently selected from a bond, a hydrocarbylene group, and a functional linker group.

27. The compound of claim 25, wherein the compound has the structure of formula (IVb) 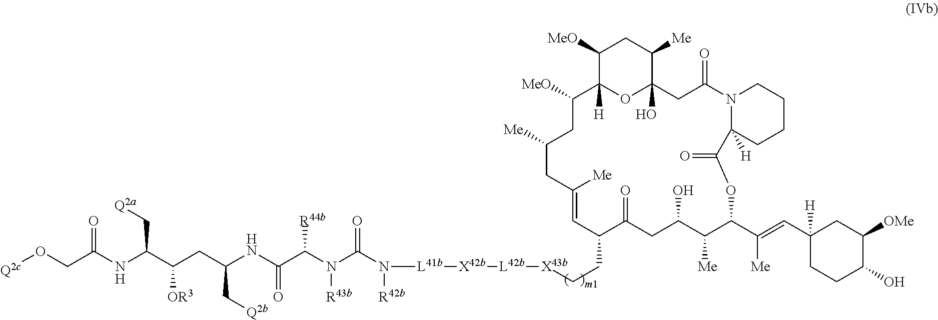
wherein, in formula (IVb):
m1 is an integer selected from 1 and 2;
R^{42b}, R^{43b}, and R^{44b} are independently selected from H and hydrocarbyl, provided that any two of R^{42b}, R^{43b}, and R^{44b} may be taken together to form a ring;
X^{42b} and X^{43b} are independently selected from a linker selected from a bond, a hydrocarbylene group, and a functional linker group; and
L^{41b}, and L^{42b} are independently selected from a bond, a hydrocarbylene group, and a functional linker group.

28. A pharmaceutical formulation comprising the compound of claim 1 and a pharmaceutically acceptable carrier.

29. (canceled)

30. A method for treating a patient with a protease inhibitor comprising administering an effective amount of the compound of claim 1 to a patient in need thereof.

31-33. (canceled)

34. The compound of claim 1, wherein the compound has an IC₅₀ value in a cell infectivity assay that is no more than twice the IC₅₀ value in a cell-free, HIV aspartyl protease inhibition assay.

35. The compound of claim 1, wherein the compound has an IC₅₀ value in a cell infectivity assay that is no more than 50 nM.

36-42. (canceled)

43. A method of synthesizing the compound of claim 1, the method comprising coupling a core fragment and an additional unit to a linker moiety.

44. (canceled)
